SEQUENCE LISTING (78 sequences)

SEQ ID NO: 1 - protein, 292 aa, Homo sapiens
MPLHVKWPFPAVPPLTWTLASSVVMGLVGTYSCFWTKYMNHLTVHNREVLYELIEKRGPATPLITVSNHQSCMDDPHLWGILKLRHIWNLKLMRWTPAAADICFTKELHSHFFSLGKCVPVCRGAEFFQAENEGKGVLDTGRHMPGAGKRREKGDGVYQKGMDFILEKLNHGDWVHIFPEGKVNMSSEFLRFKWGIGRLIAECHLNPIILPLWHVGMNDVLPNSPPYFPRFGQKITVLIGKPFSALPVLERLRAENKSAVEMRKALTDFIQEEFQHLKTQAEQLHNHLQPGR

SEQ ID NO: 2 - protein, 262 aa, Homo sapiens
MPLHVKWPFPAVPPLTWTLASSVVMGLVGTYSCFWTKYMNHLTVHNREVLYELIEKRGPATPLITVSNHQSCMDDPHLWGILKLRHIWNLKLMRWTPAAADICFTKELHSHFFSLGKCVPVCRGDGVYQKGMDFILEKLNHGDWVHIFPEGKVNMSSEFLRFKWGIGRLIAECHLNPIILPLWHVGMNDVLPNSPPYFPRFGQKITVLIGKPFSALPVLERLRAENKSAVEMRKALTDFIQEEFQHLKTQAEQLHNHLQPGR

SEQ ID NO: 3 - DNA, 879 bp, Homo sapiens
atgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagcagaatttttccaagcagagaatgaggggaaaggtgttctagacacaggcaggcacatgccaggtgctggaaaaagaagagagaaaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagatag

SEQ ID NO: 4 - DNA, 789 bp, Homo sapiens
atgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagatag

SEQ ID NO: 5 - DNA, 879 bp, Homo sapiens
atgcctctgcacgtgaagtggccgttccccgcggtgccgccgctcacgtggaccctcgccagcagcgtcgtcatgggcttggtgggcacctacagctgcttctggaccaagtacatgaaccacctgaccgtgcacaacagggaggtgctgtacgagctcatcgagaagcgaggccccgccacgcccctcatcaccgtgtccaatcaccagtcctgcatggacgaccctcatctctgggggattctgaaactccgccacatctggaacctgaagttgatgcgttggacccctgctgctgctgacatctgcttcaccaaggagctacactcccacttcttcagcttgggcaagtgtgtgcctgtgtgccgaggagcagaatttttccaagcagagaatgaggggaaaggtgtactagacacaggcaggcacatgccaggtgctggaaaaagaagagagaaaggagatggcgtctaccagaaggggatggacttcattttggagaagctcaaccacggggactgggtgcatatcttcccagaagggaaagtgaacatgagttccgaatttctgcgtttcaagtggggaatcgggcgcctgattgctgagtgtcatctcaaccccatcatcctacccctgtggcatgtcggaatgaatgacgtccttcctaacagtccgccctacttcccccgctttggacagaaaatcactgtgctgatcgggaagcccttcagtgccctgcctgtactcgagcggctccgagcggagaacaagtcggctgtggagatgcggaaagccctgacggacttcattcaagaggaatttcagcatctgaagactcaggcagagcagctccacaaccacctccagccagggagatag

SEQ ID NO: 6 - DNA, 789 bp, Homo sapiens
atgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggattctgaaactccgccacatctggaacctgaagttgatgcgttggaccccagcagcagcagacatctgcttcaccaaggagctacactcccacttcttcagcttgggcaagtgtgtgcctgtgtgccgaggagatggcgtctaccagaaggggatggacttcattttggagaagctcaaccacggggactgggtgcatatcttcccagaagggaaagtgaacatgagttccgagttcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactggagcggctccgggcggagaacaagtcggctgtggagatgcggaaagccctgacggacttcattcaagaggagttccagcatctgaagactcaggcagagcagctccacaaccacctccagccagggagatag

SEQ ID NO: 7 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 735 aa, Artificial Sequence [sequence omitted]

SEQ ID NO: 9 - protein, 736 aa, Artificial Sequence
  Synthetic
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - protein, 734 aa, Artificial Sequence
  Synthetic
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 11 - protein, 724 aa, Artificial Sequence
  Synthetic
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 14 - protein, 737 aa, Artificial Sequence
  Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 15 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 16 - protein, 736 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 18 - protein, 738 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 19 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 20 - protein, 737 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - protein, 735 aa, Artificial Sequence
  Synthetic
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 735 aa, Artificial Sequence
  Synthetic
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKLASFITQYSTGQVSVEIEWELQKENSKRWNPEIQYTSNYNKSVNVDFTVDTNGVYSEPRPIGTRYLTRNL

SEQ ID NO: 23 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLARAVFQAKKRVLEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQTVGRSSFYCLEYFPSQMLRTGNNFTFSYTFEDVPFHSSYAHSQSLDRLMNPLIDQYLYYLSRTNTPSGTTTQSRLRFSQAGASDIRDQSRNWLPGPCYRQQRVSKTAADNNNSDYSWTGATKYHLNGRDSLVNPGTAMASHKDDEEKYFPQSGVLIFGKQDSGKTNVDIERVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - RNA, 879 nt, Homo sapiens
augccucugcacgugaaguggccguuccccgcggugccgccgcucaccuggacccuggccagcagcgucgucaugggcuuggugggcaccuacagcugcuucuggaccaaguacaugaaccaccugaccgugcacaacagggaggugcuguacgagcucaucgagaagcgaggcccggccacgccccucaucaccguguccaaucaccaguccugcauggacgacccucaucucugggggauccugaaacuccgccacaucuggaaccugaaguugaugcguuggaccccugcagcugcagacaucugcuucaccaaggagcuacacucccacuucuucagcuugggcaagugugugccugugugccgaggagcagaauuuuuccaagcagagaaugaggggaaagguguucuagacacaggcaggcacaugccaggugcuggaaaaagaagagagaaaggagauggcgucuaccagaaggggauggacuucauuuuggagaagcucaaccauggggacugggugcauaucuucccagaagggaaagugaacaugaguuccgaauuccugcguuucaaguggggaaucgggcgccugauugcugagugucaucucaaccccaucauccugccccuguggcaugucggaaugaaugacguccuuccuaacaguccgcccuacuucccccgcuuuggacagaaaaucacugugcugaucgggaagcccuucagugcccugccuguacucgagcggcuccgggcggagaacaagucggcuguggagaugcggaaagcccugacggacuucauucaagaggaauuccagcaucugaagacucaggcagagcagcuccacaaccaccuccagccugggagauag

SEQ ID NO: 25 - RNA, 789 nt, Homo sapiens
augccucugcacgugaaguggccguuccccgcggugccgccgcucaccuggacccuggccagcagcgucgucaugggcuuggugggcaccuacagcugcuucuggaccaaguacaugaaccaccugaccgugcacaacagggaggugcuguacgagcucaucgagaagcgaggcccggccacgccccucaucaccguguccaaucaccaguccugcauggacgacccucaucucugggggauccugaaacuccgccacaucuggaaccugaaguugaugcguuggaccccugcagcugcagacaucugcuucaccaaggagcuacacucccacuucuucagcuugggcaagugugugccugugugccgaggagauggcgucuaccagaaggggauggacuucauuuuggagaagcucaaccauggggacugggugcauaucuucccagaagggaaagugaacaugaguuccgaauuccugcguuucaaguggggaaucgggcgccugauugcugagugucaucucaaccccaucauccugccccuguggcaugucggaaugaaugacguccuuccuaacaguccgcccuacuucccccgcuuuggacagaaaaucacugugcugaucgggaagcccuucagugcccugccuguacucgagcggcuccgggcggagaacaagucggcuguggagaugcggaaagcccugacggacuucauucaagaggaauuccagcaucugaagacucaggcagagcagcuccacaaccaccuccagccugggagauag

SEQ ID NO: 26 - RNA, 879 nt, Homo sapiens
augccucugcacgugaaguggccguuccccgcggugccgccgcucacguggacccucgccagcagcgucgucaugggcuuggugggcaccuacagcugcuucuggaccaaguacaugaaccaccugaccgugcacaacagggaggugcuguacgagcucaucgagaagcgaggccccgccacgccccucaucaccguguccaaucaccaguccugcauggacgacccucaucucugggggauucugaaacuccgccacaucuggaaccugaaguugaugcguuggaccccugcugcugcugacaucugcuucaccaaggagcuacacucccacuucuucagcuugggcaagugugugccugugugccgaggagcagaauuuuuccaagcagagaaugaggggaaagguguacuagacacaggcaggcacaugccaggugcuggaaaaagaagagagaaaggagauggcgucuaccagaaggggauggacuucauuuuggagaagcucaaccacggggacugggugcauaucuucccagaagggaaagugaacaugaguuccgaauuucugcguuucaaguggggaaucgggcgccugauugcugagugucaucucaaccccaucauccuaccccuguggcaugucggaaugaaugacguccuuccuaacaguccgcccuacuucccccgcuuuggacagaaaaucacugugcugaucgggaagcccuucagugcccugccuguacucgagcggcuccgagcggagaacaagucggcuguggagaugcggaaagcccugacggacuucauucaagaggaauuucagcaucugaagacucaggcagagcagcuccacaaccaccuccagccagggagauag

SEQ ID NO: 27 - RNA, 789 nt, Homo sapiens
augccucugcacgugaaguggccguuccccgcggugccgccgcucaccuggacccuggccagcagcgucgucaugggcuuggugggcaccuacagcugcuucuggaccaaguacaugaaccaccugaccgugcacaacagggaggugcuguacgagcucaucgagaagcgaggcccggccacgccccucaucaccguguccaaucaccaguccugcauggacgacccucaucucugggggauucugaaacuccgccacaucuggaaccugaaguugaugcguuggaccccagcagcagcagacaucugcuucaccaaggagcuacacucccacuucuucagcuugggcaagugugugccugugugccgaggagauggcgucuaccagaaggggauggacuucauuuuggagaagcucaaccacggggacugggugcauaucuucccagaagggaaagugaacaugaguuccgaguuccugcguuucaaguggggaaucgggcgccugauugcugagugucaucucaaccccaucauccugccccuguggcaugucggaaugaaugacguccuuccuaacaguccgcccuacuucccccgcuuuggacagaaaaucacugugcugaucgggaagcccuucagugcccugccuguacuggagcggcuccgggcggagaacaagucggcuguggagaugcggaaagcccugacggacuucauucaagaggaguuccagcaucugaagacucaggcagagcagcuccacaaccaccuccagccagggagauag

SEQ ID NO: 28 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQQNTAPATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - DNA, 168 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 30 - DNA, 106 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgg

SEQ ID NO: 31 - DNA, 20 bp, Artificial Sequence
  Synthetic
cttgcccactgctcacaaac

SEQ ID NO: 32 - DNA, 20 bp, Artificial Sequence
  Synthetic
caggcacatggtcctgtttc

SEQ ID NO: 33 - DNA, 20 bp, Artificial Sequence
  Synthetic
ccaagttgctagcccacaag

SEQ ID NO: 34 - DNA, 19 bp, Artificial Sequence
  Synthetic
ccatggggactgggtgcac

SEQ ID NO: 35 - DNA, 20 bp, Artificial Sequence
  Synthetic
tctgcctgcatcttcagccg

SEQ ID NO: 36 - DNA, 24 bp, Artificial Sequence
  Synthetic
attggacggctgattgctgagtgt

SEQ ID NO: 37 - DNA, 24 bp, Artificial Sequence
  Synthetic
agtctgtgagggctttccgcatct

SEQ ID NO: 38 - DNA, 18 bp, Artificial Sequence
  Synthetic
gagaacaagtcggctgtg

SEQ ID NO: 39 - DNA, 18 bp, Artificial Sequence
  Synthetic
ggctggaggtggttgtgg

SEQ ID NO: 40 - DNA, 24 bp, Artificial Sequence
  Synthetic
aaccagagtttgagtgacagaatg

SEQ ID NO: 41 - DNA, 18 bp, Artificial Sequence
  Synthetic
actccgtgcggatgtcaa

SEQ ID NO: 42 - DNA, 23 bp, Artificial Sequence
  Synthetic
gcgactcaaaaagaaggactttg

SEQ ID NO: 43 - DNA, 20 bp, Artificial Sequence
  Synthetic
ggcttgctcatcctcaatcc

SEQ ID NO: 44 - DNA, 20 bp, Artificial Sequence
  Synthetic
gcttccaggccatattggag

SEQ ID NO: 45 - DNA, 19 bp, Artificial Sequence
  Synthetic
gggggcatgacctcatctt

SEQ ID NO: 46 - DNA, 21 bp, Artificial Sequence
  Synthetic
gaggtcactcctatcctctgg

SEQ ID NO: 47 - DNA, 22 bp, Artificial Sequence
  Synthetic
gccatttcctccgacttttctc

SEQ ID NO: 48 - DNA, 22 bp, Artificial Sequence
  Synthetic
cgaagactacagttctgccatt

SEQ ID NO: 49 - DNA, 22 bp, Artificial Sequence
  Synthetic
gacgtttcagaggttctcagag

SEQ ID NO: 50 - DNA, 19 bp, Artificial Sequence
  Synthetic
tgaaacccccagccataac

SEQ ID NO: 51 - DNA, 19 bp, Artificial Sequence
  Synthetic
gggtgcccaaagaatcaga

SEQ ID NO: 52 - DNA, 18 bp, Artificial Sequence
  Synthetic
gcccccttcgacctgaca

SEQ ID NO: 53 - DNA, 20 bp, Artificial Sequence
  Synthetic
cggaagcgtggataagatgc

SEQ ID NO: 54 - DNA, 22 bp, Artificial Sequence
  Synthetic
ctcaaaacgcctaatcaacaac

SEQ ID NO: 55 - DNA, 20 bp, Artificial Sequence
  Synthetic
tacggctccagctcatagtg

SEQ ID NO: 56 - DNA, 20 bp, Artificial Sequence
  Synthetic
aaaggagccaaaaagtcacg

SEQ ID NO: 57 - DNA, 20 bp, Artificial Sequence
  Synthetic
aacggcgtgagttacaaaca

SEQ ID NO: 58 - DNA, 19 bp, Artificial Sequence
  Synthetic
atggcggcctttaggatgg

SEQ ID NO: 59 - DNA, 19 bp, Artificial Sequence
  Synthetic
tccggtctcactagctgct

SEQ ID NO: 60 - DNA, 22 bp, Artificial Sequence
  Synthetic
tggaaaatggttcgagagtcag

SEQ ID NO: 61 - DNA, 23 bp, Artificial Sequence
  Synthetic
cattccgtctctaggttaaagcg

SEQ ID NO: 62 - DNA, 20 bp, Artificial Sequence
  Synthetic
agaactggacccggttacca

SEQ ID NO: 63 - DNA, 20 bp, Artificial Sequence
  Synthetic
cacttcgctgatacccctga

SEQ ID NO: 64 - DNA, 22 bp, Artificial Sequence
  Synthetic
ttacggttccctaaacttcacg

SEQ ID NO: 65 - DNA, 21 bp, Artificial Sequence
  Synthetic
gtcacgggcaaccttttacga

SEQ ID NO: 66 - DNA, 19 bp, Artificial Sequence
  Synthetic
gctcctcttaggggccact

SEQ ID NO: 67 - DNA, 19 bp, Artificial Sequence
  Synthetic
ccacgtctcaccattgggg

SEQ ID NO: 68 - DNA, 23 bp, Artificial Sequence
  Synthetic
ctgtaacatggaaactggggaaa

SEQ ID NO: 69 - DNA, 23 bp, Artificial Sequence
  Synthetic
ccatagctgaactgaaaaccacc

SEQ ID NO: 70 - DNA, 20 bp, Artificial Sequence
  Synthetic
cttgcccactgctcacaaac

SEQ ID NO: 71 - DNA, 20 bp, Artificial Sequence
  Synthetic
caggcacatggtcctgtttc

SEQ ID NO: 72 - DNA, 20 bp, Artificial Sequence
  Synthetic
ccaagttgctagcccacaag

SEQ ID NO: 73 - DNA, 26 bp, Artificial Sequence
  Synthetic
atgacagacagatccctcctatctcc

SEQ ID NO: 74 - DNA, 24 bp, Artificial Sequence
  Synthetic
ctcatcactcgttgcatcatcgac

SEQ ID NO: 75 - DNA, 22 bp, Artificial Sequence
  Synthetic
ctcaaggctttcacgcagccac

SEQ ID NO: 76 - DNA, 29 bp, Artificial Sequence
  Synthetic
ggcatgaacatggttagcagaggctctag

SEQ ID NO: 77 - protein, 6 aa, Artificial Sequence
  Synthetic
RGNRQA

SEQ ID NO: 78 - protein, 6 aa, Artificial Sequence
  Synthetic
QQNTAP

(19) United States
(12) Patent Application Publication
(10) Pub. No.: US 2023/0211015 A1
(43) Pub. Date: Jul. 6, 2023

(54) TAZ GENE OR ENZYME REPLACEMENT THERAPY

(71) Applicant: Children's Medical Center Corporation, Boston, MA (US)

(72) Inventors: William T. Pu, Chestnut Hill, MA (US); Suya Wang, Brighton, MA (US)

(73) Assignee: Children's Medical Center Corporation, Boston, MA (US)

(21) Appl. No.: 17/799,418

(22) PCT Filed: Feb. 12, 2021
(86) PCT No.: PCT/US2021/017875

§ 371 Date: Aug. 12, 2022

Related U.S. Application Data
(60) Provisional application No. 62/976,936, filed on Feb. 14, 2020.

(51) Int. Cl. A61K 48/00; C12N 15/86; C12N 9/10; A61P 9/04

(52) U.S. Cl. CPC A61K 48/005; A61P 9/04; C12N 9/1029; C12N 15/86; A61K 38/00; C12N 2750/14143

(57) ABSTRACT

Provided herein, in some aspects, are compositions and methods for treating Barth syndrome (BTHS) using human tafazzin gene therapy or enzyme replacement therapy. The present disclosure, in some aspects, provides compositions and methods (e.g., gene therapy or enzyme replacement therapy) for treating Barth syndrome (BTHS). It was demonstrated herein that certain human Tafazzin (hTAZ) isoforms and the full length protein, as well as nucleic acids encoding them, are effective in treating BTHS.

### RELATED APPLICATION

This Application claims the benefit under 35 U.S.C. § 119(e) of U.S. Provisional Application No. 62/976,936, entitled “TAZ GENE OR ENZYME REPLACEMENT THERAPY” filed on Feb. 14, 2020, the entire contents of which are incorporated herein by reference.

### GOVERNMENT LICENSE RIGHTS

This invention was made with government support under Grant No. HL128694, awarded by The National Institutes of Health. The government has certain rights in the invention.

### BACKGROUND

Barth syndrome (BTHS) is an X-linked genetic disease that is potentially lethal. Mutation of the gene Tafazzin (TAZ), which is required for the normal biogenesis of cardiolipin (CL), ultimately leads to BTHS.

### SUMMARY

The present disclosure, in some aspects, provides compositions and methods (e.g., gene therapy or enzyme replacement therapy) for treating Barth syndrome (BTHS). It was demonstrated herein that certain human Tafazzin (hTAZ) isoforms and the full length protein, as well as nucleic acids encoding them, are effective in treating BTHS.

Accordingly, some aspects of the present disclosure provide nucleic acid molecules comprising a nucleotide sequence encoding a human Tafazzin (hTAZ) isoform comprising an amino acid sequence that is at least 90% identical to SEQ ID NO: 2. In some embodiments, the hTAZ isoform comprises the amino acid sequence of SEQ ID NO: 2.

In some embodiments, the nucleotide sequence encoding the hTAZ isoform is operably linked to a promoter.

In some embodiments, the nucleic acid molecule is a vector. In some embodiments, the vector is a viral vector for expressing the hTAZ isoform. In some embodiments, the viral vector is selected from a lentiviral vector, a retroviral vector, or a recombinant adeno-associated virus (rAAV) vector. In some embodiments, the viral vector is a rAAV vector further comprising two AAV inverted terminal repeats (ITRs) flanking the nucleotide sequence encoding the hTAZ isoform and the promoter.

In some embodiments, the nucleotide sequence encoding the hTAZ isoform is at least 90% identical to SEQ ID NO: 4. In some embodiments, the nucleotide sequence encoding the hTAZ isoform comprises SEQ ID NO: 4.

In some embodiments, the nucleotide sequence encoding the hTAZ isoform is codon-optimized. In some embodiments, the nucleotide sequence encoding the hTAZ isoform is at least 90% identical to SEQ ID NO: 6. In some embodiments, the nucleotide sequence encoding the hTAZ isoform comprises SEQ ID NO: 6.

In some embodiments, the nucleic acid is a messenger RNA (mRNA). In some embodiments, the mRNA is a modified mRNA. In some embodiments, the mRNA comprises a nucleotide sequence that is at least 90% identical to SEQ ID NO: 27. In some embodiments, the mRNA comprises the nucleotide sequence of SEQ ID NO: 27.

Other aspects of the present disclosure provide recombinant adeno-associated viruses (rAAVs) comprising a capsid protein and any one of the rAAV vectors described herein.

In some embodiments, the capsid protein is of a serotype selected from AAV1, AAV2, AAV2i8, AAV3, AAV4, AAV5, AAV6, AAV6.2, AAV7, AAV8, AAV9, AAV.rh8, AAV.rh10, AAV.rh39, AAV.43, AAV.rh74, AAV2/2-66, AAV2/2-84, and AAV2/2-125, or a variant thereof. In some embodiments, the capsid protein is of serotype AAV9 or AAV2i8. In some embodiments, the capsid protein comprises the sequence set forth in any one of SEQ ID NOs: 7-23 and 28.

In some embodiments, the rAAV is a self-complementary AAV (scAAV).

Further provided herein are compositions comprising any one of the nucleic acid molecules, any one of the rAAVs described herein. In some embodiments, the composition further comprises a pharmaceutically acceptable carrier.

Uses of any one of the nucleic acid molecules, any one of the rAAVs, or any one of the compositions described herein for treating Barth syndrome (BTHS), for improving cardiac or skeletal muscle function, or for enhancing cardiolipin biogenesis are also provided.

Accordingly, other aspects of the present disclosure provide methods of treating Barth syndrome (BTHS), methods of improving cardiac or skeletal muscle function, methods of treating cardiac or skeletal muscle diseases, or methods of enhancing cardiolipin biogenesis, the method comprising administering to a subject in need thereof an effective amount of the hTAZ isoform comprising an amino acid sequence that is at least 90% identical to SEQ ID NO: 2, any one of the nucleic acid molecules described herein, any one of the rAAVs described herein, or any one of the compositions described herein.

In some embodiments, the subject is human. In some embodiments, the administering is via injection. In some embodiments, the hTAZ isoform comprises the amino acid sequence of SEQ ID NO: 2.

In some embodiments, the hTAZ isoform is administered. In some embodiments, the nucleic acid molecule is administered. In some embodiments, the rAAV is administered. In some embodiments, the composition is administered.

In some embodiments, any one of the methods described herein comprises administering to a subject in need thereof an effective amount of a recombinant adeno-associated virus (rAAV), wherein the AAV comprises a capsid protein of serotype AAV9 and a nucleotide sequence encoding a human Tafazzin (hTAZ) isoform comprising the amino acid sequence of SEQ ID NO: 2, wherein the nucleotide sequence comprises SEQ ID NO: 6 and is operably linked to a promoter, and wherein the nucleotide sequence and the promoter are flanked by AAV inverted terminal repeats (ITRs). In some embodiments, the rAAV is a self-complementary recombinant adeno-associated virus (scAAV).

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A to 1I. Characterization of constitutive TAZ knockout mice. TAZ is located on the X-chromosome. FIG. 1A: Schematic of TAZ wild-type, floxed, and deleted alleles. FIG. 1B: PCR genotyping of TAZ mutant and wild-type mice. FIG. 1C: Loss of TAZ protein in TAZ-KO. Capillary immunoblotting of cardiac tissue from TAZ-KO and wild-type mice. FIG. 1D: Survival of TAZ-KO mice in C57BL6/J strain background. TAZ-KO (Δ/Y) mice were present at below the expected Mendalian ratio from birth. Sample sizes are indicated to the right. FIG. 1E: Survival curve for live born control and TAZ-KO mice. Shading indicates 95% confidence interval. Mantel-Cox P-value is shown. FIG. 1F: Cardiac contraction evaluated by echocardiography at P1. FS, fractional shortening. t-test. FIG. 1G: Survival curve for live born mice, with TAZ-KO mice classified by body weight at P1. Shading, 95% confidence interval. Mantel-Cox P-value between TAZ-KO groups is shown. FIG. 1H: Photograph of TAZ-KO and control littermates at P14. Bar=1 cm. FIG. 1I: Cardiac MLCL/CL ratio in control and TAZ-KO mice at 2 months and 6 months, as measured by mass spectrometry. MLCL is monolysocardiolipin, and immature form of cardiolipin. The ratio is a clinically used diagnostic of Barth syndrome, where an elevated ratio indicates disease.

FIGS. 2A to 2J. Cardiac phenotype of TAZ-KO mice. FIG. 2A: H&E stained sections demonstrating thin-walled, dilated LV of TAZ-KO at 6 months-of-age. Bar = 1 mm. FIG. 2B: Echocardiography of TAZ-KO and control mice. FS%, fractional shortening. Two-way ANOVA with Tukey multiple comparison correction. *, P<0.05. ***, P<0.001. FIG. 2C: Echocardiography of TAZ-KO and control mice. LVEDD, LV end diastolic diameter. Two-way ANOVA with Tukey multiple comparison correction. *, P<0.05. FIG. 2D: TAZ-KO cardiac fibrosis. Heart sections were stained with fast green/sirius red. Bar = 200 µm. FIG. 2E: Fibrotic area (dark gray) was quantified as a fraction of myocardial area (light gray). n=3. FIG. 2F: Cardiomyocyte (CM) apoptosis, measured by TUNEL labeling. Apoptotic CMs were TUNEL-positive, examples are indicated by arrowheads. Insets show TUNEL+ signal overlapping with DAPI (nuclei) and cardiac marker TNNI3. Bar = 200 µm. FIG. 2G: Quantification of TUNEL+ CMs. Mann-Whitney test. ***, P<0.001. FIG. 2H: Electron micrographs of cardiac mitochondria. Bar = 200 nm. FIG. 2I: Quantification of mitochondrial cross-sectional area. n=3 per group. Violin plots: shapes represent sample distribution. Dashed line, median. Dotted lines, quartiles. Numbers next to violin plots indicate number of mitochondria analyzed from at least 3 different mice per group. Mann-Whitney test. ****, P<0.0001. FIG. 2J: Evaluation of markers of heart failure, and genes critical for mitochondrial function and morphology. t-test with Holm-Sidek correction. *, P<0.05; **, P<0.01; ***, P<0.001. ns, not significant.

FIGS. 3A to 3M. Phenotype of CM-restricted TAZ-KO mice (TAZ-CKO). FIG. 3A: Schematic of cardiomyocyte-specific deletion by Myh6-Cre. FIG. 3B: Expression of TAZ protein in Ctrl vs. TAZ-CKO heart at P1, P14 and 2 months-of-age. Capillary immunoblotting was used to detect TAZ protein. FIG. 3C: MLCL/CL ratio in the hearts of Ctrl and TAZ-CKO mice evaluated at 2 months of age. t-test. P<0.001. FIG. 3D: Normal survival of TAZ-CKO mice. Mantel-Cox test. FIG. 3E: Cardiac contraction fractional shortening (FS) evaluated by echocardiography. Two way ANOVA followed by Tukey’s multiple comparison test. **, P<0.01. FIG. 3F: LV end diastolic diameter (LVEDD) evaluated by echocardiography. Two way ANOVA followed by Tukey’s multiple comparison test. **, P<0.01. FIG. 3G: Ratio of heart weight to body weight evaluated at 6 months. t-test. **, P<0.01. FIG. 3H: Heart sections derived from Ctrl and TAZ-CKO mice at 6 months were stained with sirius red/ fast green. Bars=200 µm. FIG. 3I: Quantification of percentage of fibrotic area (dark gray) in the myocardium (light gray). t-test. *, P<0.05. FIG. 3J: Evaluation of expression of heart failure markers by qRT-PCR. t-test with Holm-Sidak multiple testing correction. *, P<0.05; **, P<0.01; ****, P<0.0001; ns, not significant. FIG. 3K: Evaluation of genes critical for mitochondrial function and morphology by qRT-PCR. t-test. FIG. 3L: Apoptotic CMs in the myocardium, which are marked by double labeling by TNNI3 (light gray) and TUNEL-positive (white), shown in insets and pointed by arrowheads. Bar = 200 µm. FIG. 3M: Quantification of apoptotic CMs. Violin plots: shapes represent sample distribution. Dashed line, median. Dotted lines, quartiles. Number by shapes indicates number of sections examined from 3 different hearts per genotype. t-test. ***, P<0.001.

FIGS. 4A to 4I. Effect of AAV-mediated TAZ replacement therapy on neonatal survival of TAZ-KO mice. FIG. 4A: Schematic of AAV-hTAZ, and scAAV-hTAZ. Luciferase-expressing AAV was used as control virus (AAV-Ctrl). FIG. 4B: Experimental design. Neonatal TAZ-KO mice were treated at P1. Survival to weaning (P28) was the primary endpoint, and echocardiography and histological parameters were secondary endpoints. FIG. 4C: Viral transduction of cardiac and skeletal muscle was evaluated 7 days after AAV injection using RNA in situ hybridization probe specific to human TAZ. Fluorescent light gray punctae represent hTAZ transcripts (right panel, first and third columns from left). Cardiomyocyte marker Actn2 was stained dark gray using a specific RNA probe (right panel, second and forth columns from left). FIG. 4D: Kaplan-Meier survival curve of TAZ-KO mice after treatment with AAV at P1. Bars indicate standard error. Mantel-Cox statistical test compared to Ctrl. ***, P<0.001. FIG. 4E: Serial echocardiography. Treated mice were not distinguishable from WT until 4 months, when the treatment groups showed reduced systolic function. Two way ANOVA followed by Tukey’s post hoc test. **, P<0.01. FIG. 4F: Serial echocardiography. Treated mice were not distinguishable from WT until 4 months, when the treatment groups showed reduced systolic function. Two way ANOVA followed by Tukey’s post hoc test. *, #, P<0.05. FIG. 4G: Myocardial sections stained by fast green/sirius red. Fibrotic tissue stained dark. Bars=200 µm. FIG. 4H: Quantification of fibrosis. One-way ANOVA with Tukey post-hoc testing. #, P<0.05. **, P<0.01. ns, not significant. FIG. 4I: Correction of MLCL/CL in P7 skeletal muscle of TAZ-KO mice by AAV or scAAV expression of hTAZ. One-way ANOVA with Tukey post-hoc testing. *, P<0.05.

FIGS. 5A to 5M. Delivery of hTAZ by AAV in juvenile TAZ-CKO mice prevented development of cardiomyopathy in a dose dependent manner. FIG. 5A: Experimental outline. FIG. 5B: Echocardiography of TAZ-CKO mice. High dose AAV-hTAZ significantly prevented loss of cardiac contractility in TAZ-CKO whereas medium dose AAV-hTAZ had inconsistent efficacy. FS, fractional shortening. Two way ANOVA followed by Tukey’s post-hoc test. *, TAZ-CKO+AAV-Ctrl vs WT. #, TAZ-CKO+high dose AAV-hTAZ vs TAZ-CKO+AAV-Ctrl. *,#, P<0.05; **,##, P<0.01; ***,###, P<0.001. FIG. 5C: Echocardiography of TAZCKO mice. High dose AAV-hTAZ significantly prevented loss of cardiac contractility in TAZ-CKO whereas medium dose AAV-hTAZ had inconsistent efficacy. LVEDD, left ventricular end diastolic diameter. Echocardiography of TAZCKO mice. High dose AAV-hTAZ significantly prevented loss of cardiac contractility in TAZ-CKO whereas medium dose AAV-hTAZ had inconsistent efficacy. FS, fractional shortening. LVEDD, left ventricular end diastolic diameter. Not significant by two way ANOVA followed by Tukey’s post-hoc test. FIG. 5D: Cardiac hypertrophy, shown by the ratio of heart weight vs. body weight, was examined 3 months after treatment. Not significant by one way ANOVA test followed by Tukey’s post-hoc test. FIG. 5E: Capillary immunoblotting of TAZ in heart extracts. AAV-hTAZ delivered human (hs) TAZ has higher molecular weight than murine (mm) TAZ. * marks a non-specific band. FIG. 5F: Cardiac cardiolipin composition measured by mass spectrometry. One-way ANOVA followed by Tukey’s multiple comparison correction. Symbols as in 5B. FIG. 5G: Transcriptional correction of genes critical for mitochondrial function. One way ANOVA test followed by Tukey’s post-hoc test. Symbols as in 5B. FIG. 5H: Expression of heart failure-related genes was altered by AAVhTAZ. One way ANOVA test followed by Tukey’s post-hoc test. Symbols as in 5B. FIG. 5I: Cardiac fibrosis measured by sirius red/fast green staining of cardiac samples at 4 months of age. FIG. 5J: Quantification is shown. Bar=200 µm. One way ANOVA test followed by Tukey’s post-hoc test. Symbols as in 5B. FIG. 5K: Cardiac apoptosis measured by TUNEL staining. Insets show TUNEL signal overlapping with TNNI3 and DAPI to identify CMs. Bar=200 µm. FIG. 5L: Percentage of TUNEL-positive CMs was quantified. Numbers indicate sections analyzed, from at least 3 different hearts per group. One way ANOVA test followed by Tukey’s post-hoc test. FIG. 5M: Legend for FIGS. 5A-5L.

FIGS. 6A to 6M. AAV-hTAZ reversal of established cardiac dysfunction in TAZ-CKO mice. FIG. 6A: Experimental outline. TAZ-CKO mice with established heart dysfunction (FS<40%, ∼ 2-month-old) were treated with medium or high doses of AAV, which were calibrated to transduce ~33% or -70% CMs. FIG. 6B: Echocardiographic measurement of LV systolic function. Shading indicates standard deviation. Two way ANOVA followed by Tukey’s multiple comparison test. *, vs Control; #, vs TAZ-CKO+AAV-Ctrl; $, vs TAZ-CKO+med.AAV-hTAZ. Color indicates the comparison group. *, #,$, P<0.05. **, ##, P<0.01, ***,### P<0.001, ****,#### P<0.0001. FIG. 6C: Echocardiographic measurement of end diastolic diameter. Shading indicates standard deviation. Two way ANOVA followed by Tukey’s multiple comparison test. Symbols as in FIG. 6B. FIG. 6D: Cardiac hypertrophy, shown by the ratio of heart weight vs. bodyweight, was examined 3 months after treatment. One way ANOVA test followed by Tukey’s multiple comparison test. Symbols as in FIG. 6B. FIG. 6E: Capillary immunoblotting of TAZ in heart extracts. AAV-hTAZ delivered human (hs) TAZ is longer than murine (mm) TAZ. FIG. 6F: Cardiac cardiolipin composition measured by mass spectrometry. One way ANOVA test followed by Tukey’s multiple comparison test. Symbols as in FIG. 6B. FIG. 6G: Transcriptional correction of genes critical for mitochondrial function and morphology. One way ANOVA test followed by Tukey’s multiple comparison test. Symbols as in FIG. 6B. FIG. 6H: Expression of heart failure-related genes was normalized by AAV-hTAZ. One way ANOVA test followed by Tukey’s multiple comparison test. Symbols as in FIG. 6B. FIG. 6I: Cardiac fibrosis measured by sirius red/ fast green staining of cardiac samples at 4 months of age. FIG. 6J: Quantification is shown. Bar=200 µm. One way ANOVA test followed by Tukey’s multiple comparison test. Symbols as in FIG. 6B. FIG. 6K: Cardiac apoptosis measured by TUNEL staining. Apoptotic CMs were identified with TNNI3 shown in insets. FIG. 6L: Percentage of TUNEL-positive CMs was quantified. Insets show TUNEL signal overlapping with TNNI3 and DAPI to identify CMs. Bar= 200 µm. Numbers indicate sections analyzed, from at least 3 different hearts per group. One way ANOVA test followed by Tukey’s multiple comparison test. FIG. 6M: Legend for FIGS. 6A-6L.

FIGS. 7A to 7L. AAV-TAZ improves cardiac function and mitochondrial morphology in established cardiomyopathy in TAZ-KO. FIG. 7A: Experimental plan and legend of FIGS. 7B to 7L. TAZ-KO mice with FS<40% at ~3 months of age were treated with no agent (control), AAV-Ctrl, or AAV-hTAZ at a high dose (~70% CM transduction). Mice were followed for 3 months by echocardiography and then hearts underwent histological and molecular studies. Samples sizes for B-D are indicated. FIG. 7B: LV systolic function measured by echocardiography. Shaded areas indicate standard deviation. Two way ANOVA followed by Tukey’s post-hoc test. *, vs. Control. #, vs. TAZ-KO+AAV-Ctrl. *,#, P<0.05; **,##, P<0.01; ***,###, P<0.001; ****, ####, P<0.0001. Color indicates comparison group. FIG. 7C: LV end diastolic diameter measured by echocardiography. Shaded areas indicate standard deviation. Two way ANOVA followed by Tukey’s post-hoc test. Symbols as in FIG. 7B. FIG. 7D: Heart weight to body weight ratio. One way ANOVA with Tukey post-hoc test. Symbols as in FIG. 7B. FIG. 7E: Histological sections stained with fast green and picrosirus red. Bar=200 µm. FIG. 7F: Results were quantified as percentage of myocardial tissue area that stained red (dark gray). One way ANOVA with Tukey post-hoc test. Symbols as in FIG. 7B. FIG. 7G: Quantification of the percentage of CMs that were undergoing apoptosis, as measured by TUNEL and TNNI3 double-staining. Numbers next to violin shapes indicate number of sections analyzed, from at least 3 hearts per group. One way ANOVA with Tukey post-hoc test. Symbols as in FIG. 7B. FIG. 7H: Capillary immunoblot of heart protein extracts probed with antibody to TAZ or GAPDH. mm, murine TAZ. hs, human TAZ. FIG. 7I: Cardiac lipids were analyzed by mass spectrometry and the ratio of MLCL/CL was quantified. n=3. One way ANOVA with Tukey post-hoc test. Symbols as in FIG. 7B. FIG. 7J: Cardiac RNA was analyzed by qRTPCR using primer specific for the indicated transcript, encoded on the mitochondrial genome. Results were normalized to Gapdh. n=3. One way ANOVA with Tukey post-hoc test. Symbols as in FIG. 7B. FIG. 7K: Electron microscopy showing mitochondrial morphology. Bar=200 nm. FIG. 7L: Quantification of mitochondrial cross-sectional area. Number by violin shapes indicates number of mitochondria measured, from at least 3 different hearts. One way ANOVA with Tukey post-hoc test.

FIGS. 8A to 8C. Weight of mice at P1 and P2. FIG. 8A: Representative control and TAZ-KO mice at P1. TAZ-KO mice were smaller compared to control littermates and typically had a hunchback (arrowhead) and more pale skin tone. Bar=1 cm. FIG. 8B: Body weight of P1 and P2 mice. Between genotypes: Unpaired t-test. Within genotype: Paired t-test. FIG. 8C: Representative spectra of cardiac lipids extracted from control and TAZ-KO mice at 2 months of age. Regions of the spectra corresponding to MLCL or CL, containing acyl chains of differing lengths and saturation, are labeled.

FIGS. 9A to 9B. Cardiac fibrosis in human BTHS patients. FIG. 9A: Human cardiac samples, obtained at the time of heart transplantation, were stained with picrosirius red/fast green. FIG. 9B: Percentage of myocardial area (light gray) occupied by staining of fibrotic tissue (dark gray) was quantified in at least 3 sections per patient sample. The experiment was repeated twice. Bar=200 µm.

FIGS. 10A to 10C. Ultrastructure of control and TAZ-KO cardiomyocytes. FIG. 10A: Abnormal sarcomere morphology in TAZ-KO cardiomyocytes. M and Z lines and the A band are labeled. M line and A band were not obvious in TAZ-KO. Bar = 500 nm. FIG. 10B: Reduced mitochondrial density in TAZ-KO cardiomyocytes. FIG. 10C: Abnormal mitochondrial organization in TAZ-KO cardiomyocytes. Bar=500 nm.

FIGS. 11A to 11P. Skeletal muscle defects in TAZ-KO mice. Quadriceps muscle sections were examined by light and electron microscopy. FIG. 11A: H&E sections at P1. Scale bars: 20 µm. FIG. 11B: Muscle fiber cross sectional area (CSA) was quantified. Numbers by violin shapes indicates number of muscle fibers analyzed, from 3 mice per group. t-test: ****, P<0.0001. FIG. 11C: WGA-stained sections at 6-months-old. Scale bars: 200 µm. FIG. 11D: Cross sectional area (CSA) of indicated number of muscle fibers from 3 mice per group were analyzed. t-test: ***, P<0.001. FIG. 11E: Muscle fibrosis. Sections were stained with picrosirus red/fast green. Scale bars: 200 µm. FIG. 11F: Fraction of muscle fibers with centrally located nuclei. Number of sections analyzed for each genotype is shown next to violin shapes. n=4 animals per group. t-test: not significant. FIG. 11G: Muscle ultrastructure as imaged by transmission electron microscopy. Scale bars: 500 nm. FIG. 11H: Higher magnification images shows mitochondrial morphology. Scale bars: 100 nm. FIG. 11I: Quantification of mitochondrial CSA. Number of mitochondria analyzed is indicated by numbers next to violin shapes. t-test: ****, P<0.0001. FIG. 11J: Mitochondrial area density. Number of EM images quantified is indicated by numbers next to violin shapes. t-test *, P<0.05. FIG. 11K: Schematic of behavior assays. FIG. 11L: Maximal running time before exhaustion (N=4 TAZ-WT vs. 3 TAZ-KO). Mantel-Cox test. *, P<0.05. FIG. 11M: Number of spontaneous movements, as well as resting time were recorded from TAZ-WT and TAZ-KO mice before exercise (running on treadmill). Time spent exploring the center or peripheral of the open field chamber was also recorded to reveal levels of anxiety and stress. t-test: not significant. FIG. 11N: Time spent in indicated activities was recorded from TAZ-WT and TAZ-KO mice before exercise (running on treadmill). Time spent exploring the center or peripheral of the open field chamber indicate levels of anxiety and stress. t-test: not significant. FIG. 11O: Number of spontaneous movements, as well as total distance traveled were recorded from TAZ-WT and TAZ-KO mice after exercise (running on treadmill). t-test: *, P<0.05, **, P<0.01. FIG. 11P: Time spent in indicated activities after exercise mice after exercise (running on treadmill). t-test: ***, P<0.001.

FIG. 12. Circulating neutrophil count in TAZ-KO mice. Absolute circulating neutrophil count was measured at 6 months old. t-test: P<0.01.

FIGS. 13A to 13G. Neonatal treatment of TAZ-KO mice with gene therapy. FIG. 13A: In vivo transduction of neonatal cardiomyocytes by equivalent doses of AAV-GFP and scAAV-GFP. scAAV-GFP showed slightly higher fraction of transduced cardiac cells at one day after injection, but differences were small thereafter. FIG. 13B: Primers specific to mTaz were used to amplify human TAZ (hTAZ) or mouse Taz (mTAZ). Standard curves were established using DNA fragment cloned from mouse cDNA or the coding region of hTAZ. FIG. 13C: Comparison of hTAZ expression after treatment of AAV-hTAZ and scAAV-hTAZ. Equivalent doses were given at P1 and expression levels in heart were measured at 4 months after treatment, using mTAZ primers and the expression of hTAZ was normalized according to B. FIG. 13D: Cardiac cardiolipin was analyzed by mass spectrometry at 4 months after treatment. One-way ANOVA with Tukey post-hoc test. *, vs. Control. #, vs. TAZ-KO+AAV-Ctrl. ****, P<0.0001. ##, P<0.01. FIG. 13E: Visualization of hTAZ-positive myocytes after AAV injection at P1. Sections were examined at 21 and 90 days after injection. hTAZ and Actn2 transcripts were visualized via RNA probes that specifically hybridized to human TAZ or Actn2, respectively. FIG. 13F: Tissue tropism of scAAV9-CAG-GFP/scAAV2i8-cTNT-GFP/scAAV2i8-MHCK7-GFP. Bar= 20 µm. FIG. 13G: Survival curves of mice receiving different AAV treatment at P1. Mantel-Cox: n.s., not significant, ***P<0.001, **** P<0.0001 vs. Ctrl AAV treated KOs.

FIGS. 14A to 14B. AAV9 CAG-hTAZ skeletal muscle cell transduction. FIG. 14A: Visualization of AAV9-CAGhTAZ transduced cells in TAZ-CKO mice using an RNA probe against hTAZ. hTAZ transcripts are shown as light gray fluorescent punctae in the images (first and third columns from left). Cardiomyocyte marker Actn2 was stained using a specific RNA probe. AAV was administered at P20. Two time points (21 days and 90 days after injection) were examined. Cardiomyocytes were identified by double labeling with Actn2 (dark gray punctae within cells, second and forth columns from left) and cell membrane was stained with WGA (light grey between cells, second and forth columns from left). At 21 days after viral injection, high dose of AAV-hTAZ transduced 74% of CMs and medium dose transduced 33% of CMs in the heart. Over time transduction efficiency remained constant in the heart. FIG. 14B: AAV-hTAZ was injected to TAZ-KO mice at 3 months-of-age with the high dose defined in TAZCKO mice. AAV-hTAZ reached similar transduction of CMs in TAZ-KO mice as in TAZ-CKO mice but transduced only 27% of skeletal muscle fibers in quadriceps 21 days after injection and even less (11%) when examined at 90 days after the treatment. Transduction efficiency remained constant in the heart between 21 and 90 days yet decreased in the skeletal muscle between these time points. Bar= 20 µm.

FIGS. 15A to 15F. Cardiac defects in adult (3 month-old) TAZ-KO mice before AAV treatment. FIG. 15A: LV end diastolic diameter evaluated by echocardiography. t-test: not significant. FIG. 15B: TAZ-KO hearts showed elevated ratio of heart weight vs. body weight. t-test: **, P<0.01. FIG. 15C: Control heart section stained with picrosirius red and fast green to evaluate levels of fibrosis. FIG. 15D: TAZ-KO heart section stained with picrosirius red and fast green to evaluate levels of fibrosis. FIG. 15E: Percentage of fibrotic myocardium (dark gray) is quantified. Bar=200 µm. t-test: **, P<0.01. FIG. 15F: CM cell death was evaluated by TUNEL staining. The percentage of apoptotic CMs was quantified. t-test: **** p<0.0001 vs. Ctrl.

FIGS. 16A to 16C. AAV-hTAZ improved expression mitochondrial genes and corrected mitochondrial morphology. FIG. 16A: Transgene expression evaluated by mouse-specific Taz primers via qRT-PCR. Expression in AAV-hTAZ treated group was corrected for different mTAZ versus hTAZ amplification efficiency using standard curves shown in FIG. 13B. Relative expression in all groups was normalized to Gapdh. Statistical difference was analyzed by one-way ANOVA. ###, P<0.001. FIG. 16B: Expression of genes that are critical for mitochondrial function and morphology were evaluated by qRT-PCR. Relative expression was normalized to Gapdh. Statistical differences were analyzed by one-way ANOVA. *, vs. Control. #, vs, TAZ-KO+AAV-Ctrl. *,#, P<0.05; **, ## P<0.01; ***, ###, P<0.001. FIG. 16C: Morphology and sub-cellular distribution of mitochondria were improved by AAV-hTAZ treatment in the heart. Scale bar=500 nm.

FIGS. 17A to 17J. AAV-hTAZ minimally improved skeletal muscle defects in TAZ-KO mice. FIG. 17A: AAV CAG-hTAZ was administered at 3 months-of-age. Skeletal muscle (quadriceps) transduction was evaluated by human-specific TAZ RNA probe. Percent of hTAZ-positive fibers were quantified at 3 wks (27%) after treatment. FIG. 17B: AAV CAG-hTAZ was administered at 3 months-of-age. Skeletal muscle (quadriceps) transduction was evaluated by human-specific TAZ RNA probe. Percent of hTAZ-positive fibers were quantified at 2 months (17%) after treatment. FIG. 17C: Expression of hTAZ measured by qRTPCR using primers specific to mTaz. Relative expression of mTaz and hTAZ were corrected for different amplification efficiency as shown in. FIG. 13B. Relative expression was normalized to Gapdh. Statistical differences were analyzed by one-way ANOVA followed by Tukey’s test and were analyzed 60 days post injection. FIG. 17F: Electron microscopy showing mitochondrial morphology. Bar=200 nm. FIG. 17G: Quantification of cross sectional area of mitochondria in three groups. Statistical differences were analyzed by one-way ANOVA followed by Tukey’s test and were analyzed 90 days post injection. FIG. 17H: Quantification of density of mitochondria in three groups. Statistical differences were analyzed by one-way ANOVA followed by Tukey’s test and were analyzed 90 days post injection. FIG. 17I: Quantification of cross sectional area of muscle fibers in quadriceps. Statistical differences were analyzed by one-way ANOVA followed by Tukey’s test and were analyzed 60 days post injection. FIG. 17J: Exhaustion tests were performed to evaluate the maximal running capacity by measuring the maximal time mice running on treadmill before exhaustion behavior was observed. The test was stopped at 840 sec. Statistical differences were analyzed by t-test and were analyzed 60 days post injection. *,# P<0.05, ** P<0.01, *** P<0.001, ****, #### P<0.0001.

FIG. 18. Cardiac function of TAZ-KO mice treated with different hTAZ isoforms. evaluated by serial echocardiography. Mice lack human full length (FL) TAZ and naturally expressed an isoform equivalent to human del5.

FIGS. 19A to 19B. Protein levels of different hTAZ isoforms in protein extract of the heart after treatment.

FIGS. 20A to 20B. Relative expression of different hTAZ transcripts in the heart after AAV-hTAZ treatment. FIG. 20A: hTAZ isoform transcript level. FIG. 20B: Level of viral genome.

FIG. 21. Detection of natural TAZ isoforms in protein extracts from human (Hm) patient hearts. Capillary western blots were proved with TAZ-specific antibody to measure TAZ expression in human myocardial samples or human-derived iPSC-CMs. DEL5 encodes human Taz protein without exon 5. FL encodes full length human Taz protein. BTHH denotes iPSC-derived CMs with a frameshift mutation identified in Barth patients. Plasmids expressing DEL5 and FL were expressed in patient-derived cells and used as controls to indicate the molecular weight of two different isoforms. Human myocardial samples are shown at right.

FIGS. 22A to 22C. Expression of two isoforms of TAZ in human derived cells using plasmid DNA as input. Human iPSC-derived CMs were transfected with expression plasmids that differed only by the presence (FL) or absence (Del5) of exon 5 in the cDNA. FIG. 22A: Levels of TAZ mRNA after transfection. FIG. 22B: Expression of DEL5 and FL protein. FIG. 22C: Level of transfected plasmids. Plasmid levels were normalized to GAPDH and compared to untransfected cells.

FIGS. 23A to 23C. Expression of two isoforms of TAZ in human derived cells using mRNA as input. Human iPSC-derived CMs were transfected with modified RNA encoding DEL5-P2A-mCherry or FL-P2A-mCherry. FIG. 23A: Fluorescent signal of mCherry, a surrogate of DEL5 or FL expression level. FIG. 23B: Expression of DEL5 and FL protein after modRNA transfection. FIG. 23C: Detection of transfected modRNA, normalized to GAPDH and compared to untransfected cells.

FIG. 24. Transcriptional expression of antioxidative defense genes after expression of DEL5 vs FL in iPSC-derived CMs. mRNA was isolated 2 days after modified RNA transfection. Expression levels were normalized to GAPDH and compared to WT group. ModDel5 was more effective than FL at normalizing expression.

FIGS. 25A to 25D. Mitochondrial respiration was evaluated in iPSC-derived CMs after transfection with modified RNA expressing DEL5 or FL TAZ. Specific aspects of mitochondrial function were measured by the oxygen consumption rate (OCR) in iPSC-derived CMs using the mitochondrial stress test kit and an Agilent Seahorses Extracellular Flux Analyzer. FIG. 25A: Measurement of basal oxygen consumption rate in iPSC-derived CMs. FIG. 25B: Measurement of proton leak in iPSC-derived CMs. FIG. 25C: Measurement of ATP production in iPSC-derived CMs. FIG. 25D: Measurement of spare respiration capacity in iPSC-derived CMs. BTHH mutant cells have altered respiration capacity, which was more effectively restored by modDEL5 than with modFL.

FIGS. 26A to 26B. Cardiac function evaluated by serial echocardiography. Cardiac specific TAZ KO mice (CKO) were used as a model. Neonatal CKO mice were treated with recombinant adeno-associated virus (AAV) expressing full length or DEL5 isoforms of human Tafazzin. Similar dosage of each virus was administered. Resulting cardiac protection is shown by comparison to AAV-Luciferase treated CKO mice. Two dosages were tested: 9E9vg/g and 3E10 vg/g. FIG. 26A: At 9E9 vg/g, AAV-DEL5 showed protective effect on cardiac function but AAV-FL failed to significantly improve heart contraction. FIG. 26B: At 3E10vg/g, both viruses protect the heart up to 3 to 4 months of age. AAV-DEL5 maintained better heart contractile function at both 4 months and 5 months of age in treated CKO mice, shown by elevated mean FS% than AAV-FL.

### DETAILED DESCRIPTION OF CERTAIN EMBODIMENTS

Barth Syndrome (BTHS) is an X-linked, potentially lethal genetic disease that affects about 1 in 0.3 to 0.4 million live births¹. Hallmarks of BTHS are cardiomyopathy, skeletal myopathy, neutropenia, growth delay, poor feeding, and organic aciduria, with cardiac disease and neutropenia being the leading causes of BTHS-related mortality^{1,2}. Over 70% of BTHS patients develop cardiomyopathy in their first year, and 14% of BTHS patients require heart transplantation¹. The skeletal myopathy results in life-altering, debilitating fatigue that severely limits activities³.

Mutation of the gene Tafazzin (TAZ) causes BTHS⁴. TAZ is a nuclear-encoded, mitochondrial protein associated with the mitochondrial inner membrane⁵. TAZ is required for the normal biogenesis of cardiolipin (CL)^{6,7}, the signature phospholipid of mitochondria. CL is synthesized in nascent form with four non-specific acyl chains and undergoes TAZ-dependent remodeling, in which the acyl chains acquire a characteristic fatty acid composition, e.g. tetralinoleoyl cardiolipin in striated muscle⁸. The characteristic fatty acid composition of mature CL promotes its association with proteins in the inner mitochondrial membrane, facilitating the formation of mitochondrial super complexes^{9,10}. At the same time, protein binding protects CL from degradation to monolysocardiolipin (MLCL), which lacks one of CL’s four fatty acid residues¹¹. TAZ mutation impairs CL remodeling and protein binding, resulting in reduced mature CL and elevated MLCL/CL ratio¹²⁻¹⁵. This change in CL composition impairs the normal function of enzymes housed within the inner mitochondrial membrane, resulting in impaired electron transport chain function¹⁰, increased production of reactive oxygen species (ROS), and inefficient ATP synthesis¹⁶.

The present disclosure, in some aspects, provides compositions and methods (e.g., gene therapy or enzyme replacement therapy) for treating Barth syndrome (BTHS). It was demonstrated herein that full length hTAZ and certain hTAZ isoforms, as well as nucleic acids encoding such, are effective in treating BHTS.

Accordingly, some aspects of the present disclosure provide nucleic acid molecules comprising a nucleotide sequence encoding a human Tafazzin (hTAZ) or an isoform thereof. As used herein, “nucleic acids” may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β- D-ribo configuration, α-LNA having an α-L-ribo configuration (a diastereomer of LNA), 2′-amino-LNA having a 2′-amino functionalization, and 2′-amino- α-LNA having a 2′-amino functionalization), ethylene nucleic acids (ENA), cyclohexenyl nucleic acids (CeNA) or chimeras or combinations thereof. The nucleic acids molecules of the present disclosure may be DNA or RNA. The skilled artisan will appreciate that, except where otherwise noted, nucleic acid sequences set forth in the present disclosure will recite “T”s in a representative DNA sequence but where the sequence represents RNA, the “T”s would be substituted for “U”s.

Human tafazzin (NCBI Gene ID: 6901) has several isoforms, for example, hTAZ isoform 1 (NP_000107.1, the longest isoform, also referred to herein as the full-length hTAZ), hTAZ isoform 2 (NP_851828.1, also referred to herein as hTAZ del5), hTAZ isoform 3 (NP_851829.1), and hTAZ isoform 4 (NP_851830.1), hTAZ isoform 5 (NP_001290394.1). The amino acid sequences of examples of hTAZ isoforms that may be used in accordance with the present discloure are provided.

hTAZ isoform 1 (NP_000107.1, SEQ ID NO: 1)
MPLHVKWPFPAVPPLTWTLASSVVMGLVGTYSCFWTKYMNHLTVHNREV
LYELIEKRGPATPLITVSNHQSCMDDPHLWGILKLRHIWNLKLMRWTPA
AADICFTKELHSHFFSLGKCVPVCRGAEFFQAENEGKGVLDTGRHMPGA
GKRREKGDGVYQKGMDFILEKLNHGDWVHIFPEGKVNMSSEFLRFKWGI
GRLIAECHLNPIILPLWHVGMNDVLPNSPPYFPRFGQKITVLIGKPFSA
LPVLERLRAENKSAVEMRKALTDFIQEEFQHLKTQAEQLHNHLQPGR

hTAZ isoform 2 (NP_851828.1, SEQ ID NO: 2)
MPLHVKWPFPAVPPLTWTLASSVVMGLVGTYSCFWTKYMNHLTVHNREV
LYELIEKRGPATPLITVSNHQSCMDDPHLWGILKLRHIWNLKLMRWTPA
AADICFTKELHSHFFSLGKCVPVCRGDGVYQKGMDFILEKLNHGDWVHI
FPEGKVNMSSEFLRFKWGIGRLIAECHLNPIILPLWHVGMNDVLPNSPP
YFPRFGQKITVLIGKPFSALPVLERLRAENKSAVEMRKALTDFIQEEFQ
HLKTQAEQLHNHLQPGR

The nucleotide sequences encoding examples of hTAZ isoforms that may be used in accordance with the present discloure are also provided.

hTAZ isoform 1
- DNA (SEQ ID NO: 3; encoding SEQ ID NO: 1)
ATGCCTCTGCACGTGAAGTGGCCGTTCCCCGCGGTGCCGCCGCTCACCTG
GACCCTGGCCAGCAGCGTCGTCATGGGCTTGGTGGGCACCTACAGCTGCT
TCTGGACCAAGTACATGAACCACCTGACCGTGCACAACAGGGAGGTGCTG
TACGAGCTCATCGAGAAGCGAGGCCCGGCCACGCCCCTCATCACCGTGTC
CAATCACCAGTCCTGCATGGACGACCCTCATCTCTGGGGGATCCTGAAAC
TCCGCCACATCTGGAACCTGAAGTTGATGCGTTGGACCCCTGCAGCTGCA
GACATCTGCTTCACCAAGGAGCTACACTCCCACTTCTTCAGCTTGGGCAA
GTGTGTGCCTGTGTGCCGAGGAGCAGAATTTTTCCAAGCAGAGAATGAGG
GGAAAGGTGTTCTAGACACAGGCAGGCACATGCCAGGTGCTGGAAAAAGA
AGAGAGAAAGGAGATGGCGTCTACCAGAAGGGGATGGACTTCATTTTGGA
GAAGCTCAACCATGGGGACTGGGTGCATATCTTCCCAGAAGGGAAAGTGA
ACATGAGTTCCGAATTCCTGCGTTTCAAGTGGGGAATCGGGCGCCTGATT
GCTGAGTGTCATCTCAACCCCATCATCCTGCCCCTGTGGCATGTCGGAAT
GAATGACGTCCTTCCTAACAGTCCGCCCTACTTCCCCCGCTTTGGACAGA
AAATCACTGTGCTGATCGGGAAGCCCTTCAGTGCCCTGCCTGTACTCGAG
CGGCTCCGGGCGGAGAACAAGTCGGCTGTGGAGATGCGGAAAGCCCTGAC
GGACTTCATTCAAGAGGAATTCCAGCATCTGAAGACTCAGGCAGAGCAGC
TCCACAACCACCTCCAGCCTGGGAGATAG

hTAZ isoform 2
- DNA (SEQ ID NO: 4; encoding SEQ ID NO: 2)
ATGCCTCTGCACGTGAAGTGGCCGTTCCCCGCGGTGCCGCCGCTCACCTG
GACCCTGGCCAGCAGCGTCGTCATGGGCTTGGTGGGCACCTACAGCTGCT
TCTGGACCAAGTACATGAACCACCTGACCGTGCACAACAGGGAGGTGCTG
TACGAGCTCATCGAGAAGCGAGGCCCGGCCACGCCCCTCATCACCGTGTC
CAATCACCAGTCCTGCATGGACGACCCTCATCTCTGGGGGATCCTGAAAC
TCCGCCACATCTGGAACCTGAAGTTGATGCGTTGGACCCCTGCAGCTGCA
GACATCTGCTTCACCAAGGAGCTACACTCCCACTTCTTCAGCTTGGGCAA
GTGTGTGCCTGTGTGCCGAGGAGATGGCGTCTACCAGAAGGGGATGGACT
TCATTTTGGAGAAGCTCAACCATGGGGACTGGGTGCATATCTTCCCAGAA
GGGAAAGTGAACATGAGTTCCGAATTCCTGCGTTTCAAGTGGGGAATCGG
GCGCCTGATTGCTGAGTGTCATCTCAACCCCATCATCCTGCCCCTGTGGC
ATGTCGGAATGAATGACGTCCTTCCTAACAGTCCGCCCTACTTCCCCCGC
TTTGGACAGAAAATCACTGTGCTGATCGGGAAGCCCTTCAGTGCCCTGCC
TGTACTCGAGCGGCTCCGGGCGGAGAACAAGTCGGCTGTGGAGATGCGGA
AAGCCCTGACGGACTTCATTCAAGAGGAATTCCAGCATCTGAAGACTCAG
GCAGAGCAGCTCCACAACCACCTCCAGCCTGGGAGATAG

In some embodiments, the nucleic acid molecule described herein comprises a nucleotide sequence encoding a full-length hTAZ comprising an amino acid sequence that is at least 80% (e.g., at least 80%, at least 85%, at least 90%, at least 95%, or at least 99%) identical to SEQ ID NO: 1. In some embodiments, the nucleic acid molecule described herein comprises a nucleotide sequence encoding a full-length hTAZ comprising an amino acid sequence that is 80%, 85%, 90%, 95%, or 99% identical to SEQ ID NO: 1. In some embodiments, the nucleic acid molecule described herein comprises a nucleotide sequence encoding a full-length hTAZ comprising the amino acid sequence of SEQ ID NO: 1.

In some embodiments, the nucleic acid molecule described herein comprises a nucleotide sequence encoding a hTAZ isoform comprising an amino acid sequence that is at least 80% (e.g., at least 80%, at least 85%, at least 90%, at least 95%, or at least 99%) identical to SEQ ID NO: 2. In some embodiments, the nucleic acid molecule described herein comprises a nucleotide sequence encoding a hTAZ isoform comprising an amino acid sequence that is 80%, 85%, 90%, 95%, or 99% identical to SEQ ID NO: 2. In some embodiments, the nucleic acid molecule described herein comprises a nucleotide sequence encoding a hTAZ isoform comprising the amino acid sequence of SEQ ID NO: 2.

In some embodiments, the nucleotide sequence encoding the full-length hTAZ is at least 80% (e.g., at least 80%, at least 85%, at least 90%, at least 95%, or at least 99%) identical to SEQ ID NO: 3. In some embodiments, the nucleotide sequence encoding the full-length hTAZ is 80%, 85%, 90%, 95%, or 99% identical to SEQ ID NO: 3. In some embodiments, the nucleotide sequence encoding the full-length hTAZ comprises SEQ ID NO: 3.

In some embodiments, the nucleotide sequence encoding the hTAZ isoform is at least (e.g., at least 80%, at least 85%, at least 90%, at least 95%, or at least 99%) identical to SEQ ID NO: 4. In some embodiments, the nucleotide sequence encoding the hTAZ isoform is 80%, 85%, 90%, 95%, or 99% identical to SEQ ID NO: 4. In some embodiments, the nucleotide sequence encoding the hTAZ isoform comprises SEQ ID NO: 4.

In some embodiments, the nucleotide sequence encoding the full-length hTAZ or the hTAZ isoform is codon-optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art - non-limiting examples include services from GeneArt (Life Technologies), DNA2.0 (Menlo Park CA) and/or proprietary methods. In some embodiments, a codon optimized sequence shares less than 95% (e.g., less than 95%, less than 90%, less than 85%, or less than 80%) sequence identity to a naturally-occurring or wild-type sequence (e.g., a naturally-occurring or wild-type nucleotide sequence encoding a full-length hTAZ or a hTAZ isoform.

The codon-optimized nucleotide sequences encoding examples of hTAZ isoforms that may be used in accordance with the present discloure are also provided.

hTAZ isoform 1 - DNA codon optimized (SEQ ID NO: 5
; encoding SEQ ID NO: 1)
ATGCCTCTGCACGTGAAGTGGCCGTTCCCCGCGGTGCCGCCGCTCACGTG
GACCCTCGCCAGCAGCGTCGTCATGGGCTTGGTGGGCACCTACAGCTGCT
TCTGGACCAAGTACATGAACCACCTGACCGTGCACAACAGGGAGGTGCTG
TACGAGCTCATCGAGAAGCGAGGCCCCGCCACGCCCCTCATCACCGTGTC
CAATCACCAGTCCTGCATGGACGACCCTCATCTCTGGGGGATTCTGAAAC
TCCGCCACATCTGGAACCTGAAGTTGATGCGTTGGACCCCTGCTGCTGCT
GACATCTGCTTCACCAAGGAGCTACACTCCCACTTCTTCAGCTTGGGCAA
GTGTGTGCCTGTGTGCCGAGGAGCAGAATTTTTCCAAGCAGAGAATGAGG
GGAAAGGTGTACTAGACACAGGCAGGCACATGCCAGGTGCTGGAAAAAGA
AGAGAGAAAGGAGATGGCGTCTACCAGAAGGGGATGGACTTCATTTTGGA
GAAGCTCAACCACGGGGACTGGGTGCATATCTTCCCAGAAGGGAAAGTGA
ACATGAGTTCCGAATTTCTGCGTTTCAAGTGGGGAATCGGGCGCCTGATT
GCTGAGTGTCATCTCAACCCCATCATCCTACCCCTGTGGCATGTCGGAAT
GAATGACGTCCTTCCTAACAGTCCGCCCTACTTCCCCCGCTTTGGACAGA
AAATCACTGTGCTGATCGGGAAGCCCTTCAGTGCCCTGCCTGTACTCGAG
CGGCTCCGAGCGGAGAACAAGTCGGCTGTGGAGATGCGGAAAGCCCTGAC
GGACTTCATTCAAGAGGAATTTCAGCATCTGAAGACTCAGGCAGAGCAGC
TCCACAACCACCTCCAGCCAGGGAGATAG

hTAZ isoform 2 - DNA codon optimized (SEQ ID NO: 6
; encoding SEQ ID NO: 2)
ATGCCTCTGCACGTGAAGTGGCCGTTCCCCGCGGTGCCGCCGCTCACCTG
GACCCTGGCCAGCAGCGTCGTCATGGGCTTGGTGGGCACCTACAGCTGCT
TCTGGACCAAGTACATGAACCACCTGACCGTGCACAACAGGGAGGTGCTG
TACGAGCTCATCGAGAAGCGAGGCCCGGCCACGCCCCTCATCACCGTGTC
CAATCACCAGTCCTGCATGGACGACCCTCATCTCTGGGGGATTCTGAAAC
TCCGCCACATCTGGAACCTGAAGTTGATGCGTTGGACCCCAGCAGCAGCA
GACATCTGCTTCACCAAGGAGCTACACTCCCACTTCTTCAGCTTGGGCAA
GTGTGTGCCTGTGTGCCGAGGAGATGGCGTCTACCAGAAGGGGATGGACT
TCATTTTGGAGAAGCTCAACCACGGGGACTGGGTGCATATCTTCCCAGAA
GGGAAAGTGAACATGAGTTCCGAGTTCCTGCGTTTCAAGTGGGGAATCGG
GCGCCTGATTGCTGAGTGTCATCTCAACCCCATCATCCTGCCCCTGTGGC
ATGTCGGAATGAATGACGTCCTTCCTAACAGTCCGCCCTACTTCCCCCGC
TTTGGACAGAAAATCACTGTGCTGATCGGGAAGCCCTTCAGTGCCCTGCC
TGTACTGGAGCGGCTCCGGGCGGAGAACAAGTCGGCTGTGGAGATGCGGA
AAGCCCTGACGGACTTCATTCAAGAGGAGTTCCAGCATCTGAAGACTCAG
GCAGAGCAGCTCCACAACCACCTCCAGCCAGGGAGATAG

In some embodiments, the codon optimized nucleotide sequence encoding the full-length hTAZ is at least (e.g., at least 80%, at least 85%, at least 90%, at least 95%, or at least 99%) identical to SEQ ID NO: 5. In some embodiments, the codon optimized nucleotide sequence encoding the full-length hTAZ is 80%, 85%, 90%, 95%, or 99% identical to SEQ ID NO: 5. In some embodiments, the codon-optimized nucleotide sequence encoding the full-length hTAZ comprises SEQ ID NO: 5.

In some embodiments, the codon optimized nucleotide sequence encoding the hTAZ isoform is at least (e.g., at least 80%, at least 85%, at least 90%, at least 95%, or at least 99%) identical to SEQ ID NO: 6. In some embodiments, the codon optimized nucleotide sequence encoding the hTAZ isoform is 80%, 85%, 90%, 95%, or 99% identical to SEQ ID NO: 6. In some embodiments, the codon-optimized nucleotide sequence encoding the hTAZ isoform comprises SEQ ID NO: 6.

In some embodiments, any one of the nucleotide sequences encoding full-length hTAZ or a hTAZ isoform (e.g., DNA sequences such as any one of SEQ ID NOs: 3-6) further comprises a Kozak seuqence at the 5′ end (e.g., immediately before the ATG start codon). In some embodiments, the Kozak sequence is a native Kozak sequence in the TAZ gene, having the sequence of GGGTGGGG.

In some embodiments, the nucleotide sequence encoding the full-length hTAZ or hTAZ isoform is operably linked to a promoter. A “promoter” is a control region of a nucleic acid at which initiation and rate of transcription of the remainder of a nucleic acid are controlled. A promoter may also contain sub-regions at which regulatory proteins and molecules, such as transcription factors, bind. Promoters of the present disclosure may be constitutive, inducible, activatable, repressible, tissue-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid. In some embodiments, the promoter is a constitutive promoter. In some embodiments, the promoter is an inducible promoter (also referred to as regulatable promoter).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter. In some embodiments, the promoter used in present disclosure is a CAG promoter (e.g., containing a CMV enhancer, a promoter and the first exon and the first intron from the chicken beta-actin gene, and a splice acceptor of the rabbit beta-globin gene, as described in Okabe et al., FEBS Lett. 1997 May 5;407(3):313-9; and Alexopoulou et al., BMC Cell Biology 9: 2, 2008, incorporated herein by reference). In some embodiments, variants of the CAG promoter, such as the “CBh promoter” described in Gray et al., Human Gene Therapy 22: 1143, 2011 (incorporated herein by reference), may be used.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, inducible promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

In some embodiments, the native promoter for hTAZ used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter is a tissue-specific promoter containing regulatory sequences that impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a αmyosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the nucleic acid molecule of the present disclosure is a messenger RNA (mRNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some preferred embodiments, an mRNA is translated in vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.” One of ordinary skill in the art would understand how to identify an mRNA sequence based on the corresponding DNA sequence.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, the mRNA described herein comprises one or more chemical modifications (e.g., comprises one or more modified nucleotides). The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties.

The mRNAs described herein, some embodiments, comprise various (more than one) different modifications. In some embodiments, a particular region of a mRNA contains one, two or more (optionally different) nucleoside or nucleotide modifications. In some embodiments, a modified mRNA, introduced to a cell or organism, exhibits reduced degradation in the cell or organism, respectively, relative to an unmodified mRNA. In some embodiments, a modified mRNA introduced into a cell or organism, may exhibit reduced immunogenicity in the cell or organism, respectively (e.g., a reduced innate response).

Modifications of polynucleotides include, without limitation, those described herein. Modified mRNAs of the present disclosure may comprise modifications that are naturally-occurring, non-naturally-occurring or the polynucleotide may comprise a combination of naturally-occurring and non-naturally-occurring modifications. The mRNAs may include any useful modification, for example, of a sugar, a nucleobase, or an internucleoside linkage (e.g., to a linking phosphate, to a phosphodiester linkage or to the phosphodiester backbone).

The mRNAs described herein, in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

In some embodiments, the modified mRNA comprises one or more modified nucleosides and nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, modified nucleobases in the modified mRNA described herein are selected from the group consisting of pseudouridine (ψ), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine , 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Any one of the mRNAs, including modified mRNAs, of the present disclosure comprises a nucleotide sequence encoding hTAZ or an isoform. The nucleotide sequences of examples of the mRNAs that may be used in accordance with the present disclosure are provided.

hTAZ isoform 1 - mRNA
(SEQ ID NO: 24; encoding SEQ ID NO: 1)
AUGCCUCUGCACGUGAAGUGGCCGUUCCCCGCGGUGCCGCCGCUCACCUG
GACCCUGGCCAGCAGCGUCGUCAUGGGCUUGGUGGGCACCUACAGCUGCU
UCUGGACCAAGUACAUGAACCACCUGACCGUGCACAACAGGGAGGUGCUG
UACGAGCUCAUCGAGAAGCGAGGCCCGGCCACGCCCCUCAUCACCGUGUC
CAAUCACCAGUCCUGCAUGGACGACCCUCAUCUCUGGGGGAUCCUGAAAC
UCCGCCACAUCUGGAACCUGAAGUUGAUGCGUUGGACCCCUGCAGCUGCA
GACAUCUGCUUCACCAAGGAGCUACACUCCCACUUCUUCAGCUUGGGCAA
GUGUGUGCCUGUGUGCCGAGGAGCAGAAUUUUUCCAAGCAGAGAAUGAGG
GGAAAGGUGUUCUAGACACAGGCAGGCACAUGCCAGGUGCUGGAAAAAGA
AGAGAGAAAGGAGAUGGCGUCUACCAGAAGGGGAUGGACUUCAUUUUGGA
GAAGCUCAACCAUGGGGACUGGGUGCAUAUCUUCCCAGAAGGGAAAGUGA
ACAUGAGUUCCGAAUUCCUGCGUUUCAAGUGGGGAAUCGGGCGCCUGAUU
GCUGAGUGUCAUCUCAACCCCAUCAUCCUGCCCCUGUGGCAUGUCGGAAU
GAAUGACGUCCUUCCUAACAGUCCGCCCUACUUCCCCCGCUUUGGACAGA
AAAUCACUGUGCUGAUCGGGAAGCCCUUCAGUGCCCUGCCUGUACUCGAG
CGGCUCCGGGCGGAGAACAAGUCGGCUGUGGAGAUGCGGAAAGCCCUGAC
GGACUUCAUUCAAGAGGAAUUCCAGCAUCUGAAGACUCAGGCAGAGCAGC
UCCACAACCACCUCCAGCCUGGGAGAUAG

hTAZ isoform 2 - mRNA
(SEQ ID NO: 25; encoding SEQ ID NO: 2)
AUGCCUCUGCACGUGAAGUGGCCGUUCCCCGCGGUGCCGCCGCUCACCUG
GACCCUGGCCAGCAGCGUCGUCAUGGGCUUGGUGGGCACCUACAGCUGCU
UCUGGACCAAGUACAUGAACCACCUGACCGUGCACAACAGGGAGGUGCUG
UACGAGCUCAUCGAGAAGCGAGGCCCGGCCACGCCCCUCAUCACCGUGUC
CAAUCACCAGUCCUGCAUGGACGACCCUCAUCUCUGGGGGAUCCUGAAAC
UCCGCCACAUCUGGAACCUGAAGUUGAUGCGUUGGACCCCUGCAGCUGCA
GACAUCUGCUUCACCAAGGAGCUACACUCCCACUUCUUCAGCUUGGGCAA
GUGUGUGCCUGUGUGCCGAGGAGAUGGCGUCUACCAGAAGGGGAUGGACU
UCAUUUUGGAGAAGCUCAACCAUGGGGACUGGGUGCAUAUCUUCCCAGAA
GGGAAAGUGAACAUGAGUUCCGAAUUCCUGCGUUUCAAGUGGGGAAUCGG
GCGCCUGAUUGCUGAGUGUCAUCUCAACCCCAUCAUCCUGCCCCUGUGGC
AUGUCGGAAUGAAUGACGUCCUUCCUAACAGUCCGCCCUACUUCCCCCGC
UUUGGACAGAAAAUCACUGUGCUGAUCGGGAAGCCCUUCAGUGCCCUGCC
UGUACUCGAGCGGCUCCGGGCGGAGAACAAGUCGGCUGUGGAGAUGCGGA
AAGCCCUGACGGACUUCAUUCAAGAGGAAUUCCAGCAUCUGAAGACUCAG
GCAGAGCAGCUCCACAACCACCUCCAGCCUGGGAGAUAG

hTAZ isoform 1 - mRNA codon optimized
(SEQ ID NO: 26; encoding SEQ ID NO: 1)
AUGCCUCUGCACGUGAAGUGGCCGUUCCCCGCGGUGCCGCCGCUCACGUG
GACCCUCGCCAGCAGCGUCGUCAUGGGCUUGGUGGGCACCUACAGCUGCU
UCUGGACCAAGUACAUGAACCACCUGACCGUGCACAACAGGGAGGUGCUG
UACGAGCUCAUCGAGAAGCGAGGCCCCGCCACGCCCCUCAUCACCGUGUC
CAAUCACCAGUCCUGCAUGGACGACCCUCAUCUCUGGGGGAUUCUGAAAC
UCCGCCACAUCUGGAACCUGAAGUUGAUGCGUUGGACCCCUGCUGCUGCU
GACAUCUGCUUCACCAAGGAGCUACACUCCCACUUCUUCAGCUUGGGCAA
GUGUGUGCCUGUGUGCCGAGGAGCAGAAUUUUUCCAAGCAGAGAAUGAGG
GGAAAGGUGUACUAGACACAGGCAGGCACAUGCCAGGUGCUGGAAAAAGA
AGAGAGAAAGGAGAUGGCGUCUACCAGAAGGGGAUGGACUUCAUUUUGGA
GAAGCUCAACCACGGGGACUGGGUGCAUAUCUUCCCAGAAGGGAAAGUGA
ACAUGAGUUCCGAAUUUCUGCGUUUCAAGUGGGGAAUCGGGCGCCUGAUU
GCUGAGUGUCAUCUCAACCCCAUCAUCCUACCCCUGUGGCAUGUCGGAAU
GAAUGACGUCCUUCCUAACAGUCCGCCCUACUUCCCCCGCUUUGGACAGA
AAAUCACUGUGCUGAUCGGGAAGCCCUUCAGUGCCCUGCCUGUACUCGAG
CGGCUCCGAGCGGAGAACAAGUCGGCUGUGGAGAUGCGGAAAGCCCUGAC
GGACUUCAUUCAAGAGGAAUUUCAGCAUCUGAAGACUCAGGCAGAGCAGC
UCCACAACCACCUCCAGCCAGGGAGAUA

hTAZ isoform 2 - mRNA codon optimized
(SEQ ID NO: 27; encoding SEQ ID NO: 2)
AUGCCUCUGCACGUGAAGUGGCCGUUCCCCGCGGUGCCGCCGCUCACCUG
GACCCUGGCCAGCAGCGUCGUCAUGGGCUUGGUGGGCACCUACAGCUGCU
UCUGGACCAAGUACAUGAACCACCUGACCGUGCACAACAGGGAGGUGCUG
UACGAGCUCAUCGAGAAGCGAGGCCCGGCCACGCCCCUCAUCACCGUGUC
CAAUCACCAGUCCUGCAUGGACGACCCUCAUCUCUGGGGGAUUCUGAAAC
UCCGCCACAUCUGGAACCUGAAGUUGAUGCGUUGGACCCCAGCAGCAGCA
GACAUCUGCUUCACCAAGGAGCUACACUCCCACUUCUUCAGCUUGGGCAA
GUGUGUGCCUGUGUGCCGAGGAGAUGGCGUCUACCAGAAGGGGAUGGACU
UCAUUUUGGAGAAGCUCAACCACGGGGACUGGGUGCAUAUCUUCCCAGAA
GGGAAAGUGAACAUGAGUUCCGAGUUCCUGCGUUUCAAGUGGGGAAUCGG
GCGCCUGAUUGCUGAGUGUCAUCUCAACCCCAUCAUCCUGCCCCUGUGGC
AUGUCGGAAUGAAUGACGUCCUUCCUAACAGUCCGCCCUACUUCCCCCGC
UUUGGACAGAAAAUCACUGUGCUGAUCGGGAAGCCCUUCAGUGCCCUGCC
UGUACUGGAGCGGCUCCGGGCGGAGAACAAGUCGGCUGUGGAGAUGCGGA
AAGCCCUGACGGACUUCAUUCAAGAGGAGUUCCAGCAUCUGAAGACUCAG
GCAGAGCAGCUCCACAACCACCUCCAGCCAGGGAGAUAG

In some embodiments, the nucleotide sequence encoding the full-length hTAZ is at least 80% (e.g., at least 80%, at least 85%, at least 90%, at least 95%, or at least 99%) identical to SEQ ID NO: 24 or SEQ ID NO: 26. In some embodiments, the nucleotide sequence encoding the full-length hTAZ is 80%, 85%, 90%, 95%, or 99% identical to SEQ ID NO: 24 or SEQ ID NO: 26. In some embodiments, the nucleotide sequence encoding the full-length hTAZ comprises SEQ ID NO: 24 or SEQ ID NO: 26.

In some embodiments, the nucleotide sequence encoding the hTAZ isoform is at least (e.g., at least 80%, at least 85%, at least 90%, at least 95%, or at least 99%) identical to SEQ ID NO: 25 or SEQ ID NO: 27. In some embodiments, the nucleotide sequence encoding the hTAZ isoform is 80%, 85%, 90%, 95%, or 99% identical to SEQ ID NO: 25 or SEQ ID NO: 27. In some embodiments, the nucleotide sequence encoding the hTAZ isoform comprises SEQ ID NO: 25 or SEQ ID NO: 27.

In some embodiments, the nucleic acid molecule of the present disclosure is a vector (e.g., a cloning vector or an expression vector). The vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

A variety of host-expression vector systems may be utilized in accordance with the present disclosure. Such host-expression systems represent vehicles by which the nucleotide sequences described herein may be produced and subsequently purified, but also represent cells which may, when transformed or transfected with the appropriate nucleotide sequences, express the hTAZ or any isoform described herein in situ. These include, but are not limited to, microorganisms such as bacteria (e.g., E. coli and B. subtilis) transformed with recombinant bacteriophage DNA, plasmid DNA or cosmid DNA expression vectors containing the nucleotide sequence encoding hTAZ or any isoform described herein; yeast (e.g., Saccharomyces pichia) transformed with recombinant yeast expression vectors containing nucleotide sequence encoding hTAZ or any isoform described herein; insect cell systems infected with recombinant virus expression vectors (e.g., baclovirus) containing the nucleotide sequence encoding hTAZ or any isoform described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing nucleotide sequence encoding hTAZ or any isoform described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In some embodiments, the vector of the present disclosure is a viral vector. In some embodiments, the viral vector is suitable for mammalian expression of the hTAZ or any isoform. Suitable viral vectors include lentiviral vectors, retroviral vectors, or a recombinant adeno-associated virus (rAAV) vectors.

A “lentiviral vector” refers to a vector derived from a lentivirus genome (e.g., HIV). Lentiviral vectors have been commonly used in gene therapy, e.g., to insert beneficial genes into a host cell or organism, or to delete or modify a gene in a host cell or organism. Lentiviral vectors are efficient vehicles for gene transfer in mammalian cells due to their capacity to stably express a gene of interest in non-dividing and dividing cells.

A “retroviral vector” refers to a vector derived from a retrovirus genome. A retroviral vector consists of proviral sequences that can accommodate the gene of interest, to allow incorporation of both into the target cells. The vector also contains viral and cellular gene promoters, such as the CMV promoter, to enhance expression of the gene of interest in the target cells. Retroviral vectors have also been commonly used in gene therapy.

A “recombinant adeno-associated virus (rAAV) vector” is typically composed of, at a minimum, a transgene (the hTAZ or any isoform according to the present disclosure) and its regulatory sequences (e.g., a promoter), and 5′ and 3′ AAV inverted terminal repeats (ITRs). The transgene may comprise, as disclosed elsewhere herein, a nucleotide sequence encoding, for example, a hTAZ (full-length or an isoform), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the rAAV vectors described herein comprises two ITRs flanking (one ITR on each end of the sequence being flanked) the nucleotide sequence encoding the hTAZ (full-length or an isoform). In some embodiments, the nucleotide sequence encoding the hTAZ (full-length or an isoform) is operably linked to a promoter and the rAAV vectors described herein comprises two ITRs flanking (one ITR on each end of the sequence being flanked) the nucleotide sequence encoding the hTAZ (full-length or an isoform) and the promoter.

In some embodiments, the ITRs are of a serotype selected from AAV1, AAV2, AAV2i8, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the rAAV vector comprises ITRs of serotype AAV2. In some embodiments, the ITR used in the rAAV vector described herein comprses the nucleotide seqeunce of:

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACG (SEQ ID NO: 29).

In some embodiments, the rAAV vector of the present disclosure is a self-complementary AAV vector (scAAV). A “self-complementary AAV vector” (scAAV) refers to a vector containing a double-stranded vector genome generated by the absence of a terminal resolution site (TR) from one of the ITRs of the AAV (e.g., as described in McCarthy (2008) Molecular Therapy 16(10):1648-1656, incorporated herein by reference). The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and AmiRNA) can serve a function similar to a mutant inverted terminal repeat (mTR) during viral genome replication, generating self-complementary AAV vector genomes. In som eembodiments, the ITR used in the scAAV vector described herein comprises the nucleotide sequence of:

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGG (SEQ ID NO: 30).

Further provided herein, in some aspects, are recombinant adeno-associated virus (rAAV) comprising a capsid protein and any one of the nucleic acid molecules described herein. In some embodiments, a “capsid protein” refer to structural proteins encoded by the CAP gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host.

In some embodiments, an AAV capsid protein is of an AAV serotype selected from the group consisting of AAV1, AAV2, AAV2i8, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, AAV.rh74, or AAV.rh43 serotype. In some embodiments, an AAV capsid protein is of an AAV9 serotype. In some embodiments, an AAV capsid protein is of an AAV2i8 serotype. Non-limiting examples of the amino acid sequences of capsid proteins are provided as SEQ ID NOs: 7-23 and 28. In some embodiments, the AAV capsid of the rAAV described herein comprises the amino acid sequence of SEQ ID NO: 8. In some embodiments, the AAV capsid of the rAAV described herein comprises the amino acid sequence of SEQ ID NO: 28.

SEQ ID NO 7: AAV-CAPSID 1
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEEVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDEDKFFPMSGVMIFGKESAGA
SNTALDNVMITDEEEIKATNPVATERFGTVAVNFQSSSTDPATGDVHAMG
ALPGMVWQDRDVYLQGPIWAKIPHTDGHFHPSPLMGGFGLKNPPPQILIK
NTPVPANPPAEFSATKFASFITQYSTGQVSVEIEWELQKENSKRWNPEVQ
YTSNYAKSANVDFTVDNNGLYTEPRPIGTRYLTRPL

SEQ ID NO 8: AAV-CAPSID 2
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGS QAVGRS SFYCLEYF
PSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNS
EYSWTGATKYHLNGRDSLVNPGPAMASHKDDEEKFFPQSGVLIFGKQGSE
KTNVDIEKVMITDEEEIRTTNPVATEQYGSVSTNLQRGNRQAATADVNTQ
GVLPGMVWQDRDVYLQGPIWAKIPHTDGHFHPSPLMGGFGLKHPPPQILI
KNTPVPANPSTTFSAAKFASFITQYSTGQVSVEIEWELQKENSKRWNPEI
QYTSNYNKSVNVDFTVDTNGVYSEPRPIGTRYLTRNL

SEQ ID NO 9: AAV-CAPSID 3B
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRNL

SEQ ID NO 10: AAV-CAPSID 4
MTDGYLPDWLEDNLSEGVREWWALQPGAPKPKANQQHQDNARGLVLPGYK
YLGPGNGLDKGEPVNAADAAALEHDKAYDQQLKAGDNPYLKYNHADAEFQ
QRLQGDTSFGGNLGRAVFQAKKRVLEPLGLVEQAGETAPGKKRPLIESPQ
QPDSSTGIGKKGKQPAKKKLVFEDETGAGDGPPEGSTSGAMSDDSEMRAA
AGGAAVEGGQGADGVGNASGDWHCDSTWSEGHVTTTSTRTWVLPTYNNHL
YKRLGESLQSNTYNGFSTPWGYFDFNRFHCHFSPRDWQRLINNNWGMRPK
AMRVKIFNIQVKEVTTSNGETTVANNLTSTVQIFADSSYELPYVMDAGQE
GSLPPFPNDVFMVPQYGYCGLVTGNTSQQQTDRNAFYCLEYFPSQMLRTG
NNFEITYSFEKVPFHSMYAHSQSLDRLMNPLIDQYLWGLQSTTTGTTLNA
GTATTNFTKLRPTNFSNFKKNWLPGPSIKQQGFSKTANQNYKIPATGSDS
LIKYETHSTLDGRWSALTPGPPMATAGPADSKFSNSQLIFAGPKQNGNTA
TVPGTLIFTSEEELAATNATDTDMWGNLPGGDQSNSNLPTVDRLTALGAV
PGMVWQNRDIYYQGPIWAKIPHTDGHFHPSPLIGGFGLKHPPPQIFIKNT
PVPANPATTFSSTPVNSFITQYSTGQVSVQIDWEIQKERSKRWNPEVQFT
SNYGQQNSLLWAPDAAGKYTEPRAIGTRYLTHHL

SEQ ID NO 11: AAV-CAPSID 5
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL

SEQ ID NO 12: AAV-CAPSID 6
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPFGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDKDKFFPMSGVMIFGKESAGA
SNTALDNVMITDEEEIKATNPVATERFGTVAVNLQSSSTDPATGDVHVMG
ALPGMVWQDRDVYLQGPIWAKIPHTDGHFHPSPLMGGFGLKHPPPQILIK
NTPVPANPPAEFSATKFASFITQYSTGQVSVEIEWELQKENSKRWNPEVQ
YTSNYAKSANVDFTVDNNGLYTEPRPIGTRYLTRPL

SEQ ID NO 13: AAV-CAPSID 6.2
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPATPAAVGP
TTMASGGGAPMADNNEGADGVGNASGNWHCDSTWLGDRVITTSTRTWALP
TYNNHLYKQISSASTGASNDNHYFGYSTPWGYFDFNRFHCHFSPRDWQRL
INNNWGFRPKRLNFKLFNIQVKEVTTNDGVTTIANNLTSTVQVFSDSEYQ
LPYVLGSAHQGCLPPFPADVFMIPQYGYLTLNNGSQAVGRSSFYCLEYFP
SQMLRTGNNFTFSYTFEDVPFHSSYAHSQSLDRLMNPLIDQYLYYLNRTQ
NQSGSAQNKDLLFSRGSPAGMSVQPKNWLPGPCYRQQRVSKTKTDNNNSN
FTWTGASKYNLNGRESIINPGTAMASHKDDKDKFFPMSGVMIFGKESAGA
SNTALDNVMITDEEEIKATNPVATERFGTVAVNLQSSSTDPATGDVHVMG
ALPGMVWQDRDVYLQGPIWAKIPHTDGHFHPSPLMGGFGLKHPPPQILIK
NTPVPANPPAEFSATKFASFITQYSTGQVSVEIEWELQKENSKRWNPEVQ
YTSNYAKSANVDFTVDNNGLYTEPRPIGTRYLTRPL

SEQ ID NO 14: AAV-CAPSID 7
MAADGYLPDWLEDNLSEGIREWWDLKPGAPKPKANQQKQDNGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPAKKRPVEPSP
QRSPDSSTGIGKKGQQPARKRLNFGQTGDSESVPDPQPLGEPPAAPSSVG
SGTVAAGGGAPMADNNEGADGVGNASGNWHCDSTWLGDRVITTSTRTWAL
PTYNNHLYKQISSETAGSTNDNTYFGYSTPWGYFDFNRFHCHFSPRDWQR
LINNNWGFRPKKLRFKLFNIQVKEVTTNDGVTTIANNLTSTIQVFSDSEY
QLPYVLGSAHQGCLPPFPADVFMIPQYGYLTLNNGSQSVGRSSFYCLEYF
PSQMLRTGNNFEFSYSFEDVPFHSSYAHSQSLDRLMNPLIDQYLYYLART
QSNPGGTAGNRELQFYQGGPSTMAEQAKNWLPGPCFRQQRVSKTLDQNNN
SNFAWTGATKYHLNGRNSLVNPGVAMATHKDDEDRFFPSSGVLIFGKTGA
TNKTTLENVLMTNEEEIRPTNPVATEEYGIVSSNLQAANTAAQTQVVNNQ
GALPGMVWQNRDVYLQGPIWAKIPHTDGNFHPSPLMGGFGLKHPPPQILI
KNTPVPANPPEVFTPAKFASFITQYSTGQVSVEIEWELQKENSKRWNPEI
QYTSNFEKQTGVDFAVDSQGVYSEPRPIGTRYLTRNL

SEQ ID NO 15: AAV-CAPSID 8
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL

SEQ ID NO 16: AAV-CAPSID 9
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL

SEQ ID NO 17: AAV-CAPSID rh8
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKTGQQPAKKRLNFGQTGDSESVPDPQPLGEPPAAPSGLGP
NTMASGGGAPMADNNEGADGVGNSSGNWHCDSTWLGDRVITTSTRTWALP
TYNNHLYKQISNGTSGGSTNDNTYFGYSTPWGYFDFNRFHCHFSPRDWQR
LINNNWGFRPKRLNFKLFNIQVKEVTTNEGTKTIANNLTSTVQVFTDSEY
QLPYVLGSAHQGCLPPFPADVFMVPQYGYLTLNNGSQALGRSSFYCLEYF
PSQMLRTGNNFQFSYTFEDVPFHSSYAHSQSLDRLMNPLIDQYLYYLVRT
QTTGTGGTQTLAFSQAGPSSMANQARNWVPGPCYRQQRVSTTTNQNNNSN
FAWTGAAKFKLNGRDSLMNPGVAMASHKDDDDRFFPSSGVLIFGKQGAGN
DGVDYSQVLITDEEEIKATNPVATEEYGAVAINNQAANTQAQTGLVHNQG
VIPGMVWQNRDVYLQGPIWAKIPHTDGNFHPSPLMGGFGLKHPPPQILIK
NTPVPADPPLTFNQAKLNSFITQYSTGQVSVEIEWELQKENSKRWNPEIQ
YTSNYYKSTNVDFAVNTEGVYSEPRPIGTRYLTRNL

SEQ ID NO 18: AAV-CAPSID rh10
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYQFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFSQAKLASFITQYSTGQVSVEIEWELQKENSKRWNPE
IQYTSNYYKSTNVDFAVNTDGTYSEPRPIGTRYLTRNL

SEQ ID NO 19: AAV-CAPSID rh39
MAADGYLPDWLEDNLSEGIREWWALKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEAAKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYTFEDVPFHSSYAHSQSLDRLMNPLIDQYLYYLSR
TQSTGGTQGTQQLLFSQAGPANMSAQAKNWLPGPCYRQQRVSTTLSQNNN
SNFAWTGATKYHLNGRDSLVNPGVAMATHKDDEERFFPSSGVLMFGKQGA
GRDNVDYSSVMLTSEEEIKTTNPVATEQYGVVADNLQQTNTGPIVGNVNS
QGALPGMVWQNRDVYLQGPIWAKIPHTDGNFHPSPLMGGFGLKHPPPQIL
IKNTPVPADPPTTFSQAKLASFITQYSTGQVSVEIEWELQKENSKRWNPE
IQYTSNYYKSTNVDFAVNTEGTYSEPRPIGTRYLTRNL

SEQ ID NO 20: AAV-CAPSID rh43
MAADGYLPDWLEDNLSEGIREWWDLKPGAPKPKANQQKQDDGRGLVLPGY
KYLGPFNGLDKGEPVNAADAAALEHDKAYDQQLEAGDNPYLRYNHADAEF
QERLQEDTSFGGNLGRAVFQAKKRVLEPLGLVEEGAKTAPGKKRPVEQSP
QEPDSSSGIGKKGQQPARKRLNFGQTGDSESVPDPQPLGEPPAAPSGVGP
NTMAAGGGAPMADNNEGADGVGSSSGNWHCDSTWLGDRVITTSTRTWALP
TYNNHLYKQISNGTSGGATNDNTYFGYSTPWGYFDFNRFHCHFSPRDWQR
LINNNWGFRPKRLSFKLFNIQVKEVTQNEGTKTIANNLTSTIQVFTDSEY
QLPYVLGSAHQGCLPPFPADVFMIPQYGYLTLNNGSQAVGRSSFYCLEYF
PSQMLRTGNNFQFTYTFEDVPFHSSYAHSQSLDRLMNPLIDQYLYYLSRT
QTTGGTANTQTLGFSQGGPNTMANQAKNWLPGPCYRQQRVSTTTGQNNNS
NFAWTAGTKYHLNGRNSLANPGIAMATHKDDEERFFPSNGILIFGKQNAA
RDNADYSDVMLTSEEEIKTTNPVATEEYGIVADNLQQQNTAPQIGTVNSQ
GALPGMVWQNRDVYLQGPIWAKIPHTDGNFHPSPLMGGFGLKHPPPQILI
KNTPVPADPPTTFNQSKLNSFITQYSTGQVSVEIEWELQKENSKRWNPEI
QYTSNYYKSTSVDFAVNTEGVYSEPRPIGTRYLTRNL

SEQ ID NO 21: AAV-CAPSID 2/2-66
MAADGYLPDWLEDTLSEGIRQWWKLKPGPPPPKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGS QAVGRS SFYCLEYF
PSQMLRTGNNFTFSYTFEDVPFHSSYAHSQSLDRLMNPLIDQYLYYLSKT
NAPSGTTTMSRLQFSQAGASDIRDQSRNWLPGPCYRQQRVSKTAADNNNS
DYSWTGATKYHLNGRDSLVNPGPAMASHKDDEEKYFPQSGVLIFGKQDSG
KTNVDIEKVMITDEEEIRTTNPVATEQYGSVSTNLQSGNTQAATTDVNTQ
GVLPGMVWQDRDVYLQGPIWAKIPHTDGHFHPSPLMGGFGLKHPPPQILI
KNTPVPANPSTTFSAAKFASFITQYSTGQVSVEIEWELQKENSKRWNPEI
QYTSNYNKSVNVDFTVDTNGVYSEPRPIGTRYLTRNL

SEQ ID NO 22: AAV-CAPSID 2/2-84
MAADGYLPDWLEDTLSEGIRQWWKLKPGPPPPKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGS QAVGRS SFYCLEYF
PSQMLRTGNNFTFSYTFEDVPFHSSYAHSQSLDRLMNPLIDQYLYYLSKT
NAPSGTTTMSRLQFSQAGASDIRDQSRNWLPGPCYRQQRVSKTAADNNNS
DYSWTGATKYHLNGRDSLVNPGPAMASHKDDEEKYFPQSGVLIFGKQDSG
KTNVDIEKVMITDEEEIRTTNPVATEQYGSVSTNLQSGNTQAATTDVNTQ
GVLPGMVWQDRDVYLQGPIWAKIPHTDGHFHPSPLMGGFGLKHPPPQILI
KNTPVPANPSTTFSAAKLASFITQYSTGQVSVEIEWELQKENSKRWNPEI
QYTSNYNKSVNVDFTVDTNGVYSEPRPIGTRYLTRNL

SEQ ID NO 23: AAV-CAPSID 2/2-125
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLARAVFQAKKRVLEPLGLVEEPVKTAPGKKRPVEHSP
AEPDSSSGTGKSGQQPARKRLNFGQTGDADSVPDPQPLGQPPAAPSGLGT
NTMASGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQTVGRSSFYCLEYFPS
QMLRTGNNFTFSYTFEDVPFHSSYAHSQSLDRLMNPLIDQYLYYLSRTNT
PSGTTTQSRLRFSQAGASDIRDQSRNWLPGPCYRQQRVSKTAADNNNSDY
SWTGATKYHLNGRDSLVNPGTAMASHKDDEEKYFPQSGVLIFGKQDSGKT
NVDIERVMITDEEEIRTTNPVATEQYGSVSTNLQSGNTQAATSDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

SEQ ID NO 28: AAV-CAPSID 2i8
(substitution of RGNRQA (amino acids 585-590)
of AAV2-CAPSID with QQNTAP)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGS QAVGRS SFYCLEYF
PSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNS
EYSWTGATKYHLNGRDSLVNPGPAMASHKDDEEKFFPQSGVLIFGKQGSE
KTNVDIEKVMITDEEEIRTTNPVATEQYGSVSTNLQQQNTAPATADVNTQ
GVLPGMVWQDRDVYLQGPIWAKIPHTDGHFHPSPLMGGFGLKHPPPQILI
KNTPVPANPSTTFSAAKFASFITQYSTGQVSVEIEWELQKENSKRWNPEI
QYTSNYNKSVNVDFTVDTNGVYSEPRPIGTRYLTRNL

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the present disclosure provides rAAV vector transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (Sf9), or a mammalian (e.g., human, rodent, non-human primate, etc.) cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

Further provided herein are compositions (e.g., pharmaceutical compositions) comprising any one of the nucleic acid molecules (e.g., vectors or mRNAs), hTAZ proteins (full-length or an isoform), or any one of the rAAVs described herein. In some embodiments, the composition further comprises a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Suitable carriers may be readily selected by one of skill in the art in view of the indication for which the composition (e.g., pharmaceutical composition) is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Typically, the compositions (e.g., pharmaceutical compositions) may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the compositions comprise any one of the rAAVs described herein. In some embodiments, these compositions are formulated to reduce aggregation of AAV particles in the composition, particularly where high rAAV concentrations are present (e.g., ~1013 GC/ml or more). Methods for reducing aggregation of rAAVs are well known in the art and, include, for example, addition of surfactants, pH adjustment, salt concentration adjustment, etc. (See, e.g., Wright FR, et al., Molecular Therapy (2005) 12, 171-178, the contents of which are incorporated herein by reference.)

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer’s solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or nonaqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington’s Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active agents in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Delivery vehicles such as liposomes, nanocapsules, microparticles, microspheres, lipid particles, vesicles, and the like, may be used for the introduction of the compositions of the present disclosure into suitable host cells. In particular, the nucleic acids, proteins, or rAAVs may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids, proteins, or the rAAVs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 µm. Sonication of MLVs results in the formation of small unilamellar vesicles (SUVs) with diameters in the range of 200 to 500 Å, containing an aqueous solution in the core.

Alternatively, nanocapsule formulations of the active agents may be used. Nanocapsules can generally entrap substances in a stable and reproducible way. To avoid side effects due to intracellular polymeric overloading, such ultrafine particles (sized around 0.1 µm) should be designed using polymers able to be degraded in vivo. Biodegradable polyalkylcyanoacrylate nanoparticles that meet these requirements are contemplated for use.

In addition to the methods of delivery described above, the following techniques are also contemplated as alternative methods of delivering the compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Other aspects of the present disclosure provide uses of any one of the nucleic acid molecule, the rAAV, or the composition described herein. In some embodiments, the nucleic acid molecule, the rAAV, or the composition described herein are used for treating Barth syndrome (BTHS). In some embodiments, the nucleic acid molecule, the rAAV, or the composition described herein are used for improving cardiac or skeletal muscle function (e.g., in a subject affected by a mutation in the TAZ gene). In some embodiments, the nucleic acid molecule, the rAAV, or the composition described herein are used for enhancing cardiolipin biogenesis (e.g., in a subject having acquired conditions where cardiolipin metabolism is perturbed, such as a subject having diabetes or heart failure).

Accordingly, some aspects of the present disclosure provide methods of treating Barth syndrome (BTHS), methods of improving cardiac and skeletal muscle function (e.g., in a subject affected by a mutation in the TAZ gene) and/or methods of enhancing cardiolipin biogenesis (e.g., in a subject having acquired conditions where cardiolipin metabolism is perturbed, such as a subject having diabetes or heart failure). In some embodiments, the method comprises administering to a subject in need thereof an effective amount of a hTAZ isoform comprising an amino acid sequence that is at least 90% identical to SEQ ID NO: 2. In some embodiments, the method comprises administering to a subject in need thereof an effective amount of the nucleic acid molecule described herein. In some embodiments, the nucleic acid is a vector (e.g., a viral vector). In some embodiments, the nucleic acid is a mRNA (e.g., modified mRNA). In some embodiments, the method comprises administering to a subject in need thereof an effective amount of the rAAV described herein. In some embodiments, the method comprises administering to a subject in need thereof an effective amount of the composition described herein.

In some embodiments, the method comprises administering to a subject in need thereof an effective amount of a recombinant adeno-associated virus (rAAV), wherein the AAV comprises a capsid protein of serotype AAV9 and a nucleotide sequence encoding a human Tafzzin (hTAZ) isoform comprising the amino acid sequence of SEQ ID NO: 2, wherein the nucleotide sequence comprises SEQ ID NO: 6 and is operably linked to a promoter, and wherein the nucleotide sequence and the promoter are flanked by AAV inverted terminal repeats (ITRs). In some embodiments, the rAAV is a self-complementary recombinant adeno-associated virus (scAAV).

In its broadest sense, the terms “treatment” or “to treat” refer to both therapeutic and prophylactic treatments. If the subject is in need of treatment of a disease (e.g., Barth syndrome), “treating the condition” refers to ameliorating, reducing or eliminating one or more symptoms associated with the or preventing any further progression of the disease (e.g., Barth syndrome). If the subject in need of treatment is one who is at risk of having Barth syndrome, then treating the subject refers to reducing the risk of the subject having Barth syndrome or preventing the subject from developing Barth syndrome.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof.

The term “therapeutically effective amount” of the present disclosure refers to the amount necessary or sufficient to realize a desired biologic effect. For example, a therapeutically effective amount of hTAZ or nucleic acid encoding such associated with the present disclosure may be that amount sufficient to ameliorate one or more symptoms of Barth syndrome. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the present disclosure without necessitating undue experimentation.

In some embodiments, an “effective amount” of an rAAV is an amount sufficient to target infect an animal, target a desired tissue (e.g., heart tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹³ to 10¹⁵ rAAV genome copies is appropriate.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., delivery to the heart), oral, inhalation (including intranasal and intratracheal delivery), intraocular, intravenous, intramuscular, subcutaneous, intradermal, intratumoral, and other parental routes of administration. Routes of administration may be combined, if desired.

The hTAZ, nucleic acids, rAAVs, and compositions comprising such of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the hTAZ, nucleic acids, rAAVs, and compositions to a mammalian subject may be by, for example, intramuscular injection or by administration into the bloodstream of the mammalian subject. Administration into the bloodstream may be by injection into a vein, an artery, or any other vascular conduit. In some embodiments, the hTAZ, nucleic acids, rAAVs, and compositions as described in the disclosure are administered by intravenous injection. In some embodiments, the hTAZ, nucleic acids, rAAVs, and compositions are administered by intramuscular injection. In some embodiments, the hTAZ, nucleic acids, rAAVs, and compositions are administered by injection into the heart.

In some embodiments, a dose of the hTAZ, nucleic acids, rAAVs, or compositions are administered to a subject by intramuscular injection no more than once per calendar day (e.g., a 24-hour period). In some embodiments, a dose of the hTAZ, nucleic acids, rAAVs, or compositions are administered by intramuscular injection to a subject no more than once per 2, 3, 4, 5, 6, or 7 calendar days. In some embodiments, a dose of the hTAZ, nucleic acids, rAAVs, or compositions is administered to a subject no more than once per calendar week (e.g., 7 calendar days). In some embodiments, a dose of the hTAZ, nucleic acids, rAAVs, or compositions is administered to a subject no more than bi-weekly (e.g., once in a two calendar week period). In some embodiments, a dose of rAAV is administered to a subject no more than once per calendar month (e.g., once in 30 calendar days). In some embodiments, a dose of the hTAZ, nucleic acids, rAAVs, or compositions is administered to a subject no more than once per six calendar months. In some embodiments, a dose of the hTAZ, nucleic acids, rAAVs, or compositions is administered to a subject no more than once per calendar year (e.g., 365 days or 366 days in a leap year). In some embodiments, a dose of the hTAZ, nucleic acids, rAAVs, or compositions is administered to a subject as single dose therapy.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1. AAV Gene Therapy Prevents and Reverses Heart Failure in a Murine Knockout Model of Barth Syndrome

Barth Syndrome (BTHS) is an X-linked, potentially lethal genetic disease that affects about 1 in 0.3 to 0.4 million live births¹. Hallmarks of BTHS are cardiomyopathy, skeletal myopathy, neutropenia, growth delay, poor feeding, and organic aciduria, with cardiac disease and neutropenia being the leading causes of BTHS-related mortality^{1,2}. In fact, over 70% of BTHS patients develop cardiomyopathy in their first year, and 14% of BTHS patients require heart transplantation¹. The skeletal myopathy results in life-altering, debilitating fatigue that severely limits activities³.

Mutation of the gene Tafazzin (TAZ) causes BTHS⁴. TAZ is a nuclear-encoded, mitochondrial protein associated with the mitochondrial inner membrane⁵. TAZ is required for the normal biogenesis of cardiolipin (CL)^{6,7}, the signature phospholipid of mitochondria. CL is synthesized in nascent form with four non-specific acyl chains and undergoes TAZ-dependent remodeling, in which the acyl chains acquire a characteristic fatty acid composition, e.g. tetralinoleoyl cardiolipin in striated muscle⁸. The characteristic fatty acid composition of mature CL promotes its association with proteins in the inner mitochondrial membrane, facilitating the formation of mitochondrial super complexes^{9,10}. At the same time, protein binding protects CL from degradation to monolysocardiolipin (MLCL), which lacks one of CL’s four fatty acid residues¹¹. TAZ mutation impairs CL remodeling and protein binding, resulting in reduced mature CL and elevated MLCL/CL ratio¹²⁻¹⁵. This change in CL composition impairs the normal function of enzymes housed within the inner mitochondrial membrane, resulting in impaired electron transport chain function¹⁰, increased production of reactive oxygen species (ROS), and inefficient ATP synthesis¹⁶.

Several experimental models of BTHS have been reported, including yeast¹⁷ and Drosophila¹⁸ knockouts, and human induced pluripotent stem cell-derived CMs¹⁶ harboring TAZ mutations. However, a TAZ knockout mouse that recapitulates the cardinal features of the human condition has been lacking. A doxycycline-induced short hairpin RNA TAZ knockdown mouse has been reported, in which high dose doxycycline leads to 80-90% TAZ protein depletion^{19,20}. However, important limitations of this model are residual TAZ expression, relatively mild cardiac involvement, high inter-animal variability, and the need for continuous, high dose doxycycline treatment, which itself impacts mitochondrial function²¹ and metalloprotease activity²².

Currently there are no targeted therapies for BTHS. Patients are treated with supportive medical management for cardiomyopathy (standard heart failure medications; transplantation) and neutropenia (GM-CSF). One attractive strategy is AAV gene therapy to replace mutant TAZ. It was previously shown that TAZ gene replacement normalizes function of BTHS human induced pluripotent stem cell-derived cardiomyocytes (iPSC-CMs)¹⁶, and AAV-TAZ was shown to partially normalize cardiac function and skeletal muscle fatigue in the TAZ knockdown model²³. While promising, this proof-of-concept study had several important caveats that limit extrapolation to clinical translation. First, the study was based on the TAZ knockdown model, which has residual TAZ expression and relatively mild disease. Second, the study did not evaluate dose-response, a key issue given failure of a recent clinical cardiac AAV gene therapy trial likely as a result of insufficient dosing²⁴. Third, the study did not evaluate the ability of AAV-TAZ to reverse established cardiac disease. Fourth, the study did not evaluate the durability of the therapeutic response.

Here the phenotype of mice with germline or cardiac-specific TAZ knockout was characterized. It was shown that these mice have substantial fetal and perinatal demise that was likely due to skeletal muscle weakness. Survivors develop progressive cardiomyopathy and cardiac fibrosis. AAV-TAZ rescued neonatal demise, prevented cardiac dysfunction, and reversed established heart disease. However, therapeutic efficacy and durability were dependent on transduction of -70% cardiomyocytes (CMs). These results establish a platform for testing of BTHS therapies and demonstrates the efficacy of TAZ gene therapy when administered at a sufficient dose.

### Material and Methods

### Animal

Animal experiments were performed under protocols approved by the Boston Children’s Hospital Institutional Animal Care and Use Committee. Mice harboring the Taz^{fl} and Taz^{Δ} allele were described previously²⁵. These alleles have been backcrossed onto C57BL/6J for over 5 generations. Myh6-Cre²⁶ mice were purchased from Jackson Laboratory and have been backcrossed to C57BL/6J for over 10 generations. Echocardiography of awake mice was performed using a VEVO 2100. Echocardiography was performed blinded to genotype and treatment group. Mice were genotyped by PCR using the primers indicated in Table 1.

### Genotyping

Genomic DNA was isolated using KAPA Express Extraction Kit (KK7102). Genotyping PCR of Taz WT allele vs. KO allele was carried out using the primers WT-U1 (5′-CTTGCCCACTGCTCACAAAC-3′), WT-D1 (5′-CAGGCACATGGTCCTGTTTC- 3′) and KO-U1 (5′-CCAAGTTGCTAGCCCACAAG- 3′), which generates products of 383 bp and 280 bp, representing WT allele and KO allele, respectively. Differentiation of Taz-floxed allele against WT allele was detected by PCR using primers WT-U1 and WT-D1, which generates a 451 bp product for the identification of floxed allele. All the genotyping PCR was performed using Go-Taq Mastermix (Promega, M7122) and shares the same thermo-program: 95° C. for 2 min; 35 cycles of 95° C. for 30 sec, 60° C. for 1 min, 72° C. for 1 min; and a final extension step of 72° C. for 5 min. Amplicon sizes were analyzed with electrophoresis using 1% agarose gel with ethidium bromide.

### AAV Vectors

AAV-TAZ and AAV-Luciferase vectors were constructed from AAV-CAG-GFP (Addgene plasmid #37825) by replacing GFP cDNA with luciferase or codon optimized human full-length Tafazzin cDNA (hTAZ), synthesized by Genewiz. AAV9 was produced by triple transfection of HEK293T cells, purified and titered as described²⁷. AAV was administered to mice less than 10 days of age by subcutaneous injection, and to older mice by retro-orbital injection. High dose and medium dose AAV doses were 2x10¹⁰ and 1x10¹⁰ vg/g, respectively.

### AAV Production

AAV was produced according to previously published protocol. Briefly, AAV was produced by HEK293T cells transfected with three plasmids carrying AAV9-Rep/Cap, target gene flanked by ITRs, and necessary adenovirus helper genes. 72 hours later HEK293T cells were lysed and the AAV-containing cell medium and lysate were both collected. AAV particles were purified by ultracentrifugation on an iodixanol-based density gradient. Purified AAV was stored at -80° C. until needed. AAV plasmids were obtained from the Penn Vector Core.

### QPCR-Based AAV Titration

Purified AAV (5 µl) was first treated with DNase I for digesting residual plasmid carried over from transfected HEK293 cells and then incubated with proteinase K for digesting the viral capsid. The viral genome (VG) was quantified using SYBR Green PCR Master Mix (Applied Biosystems, Cat# 4367659) with two primers flanking a 170bp region in the CAG promoter. Standard curve was established using serial dilution of the amplicon DNA with known concentration as the input of QPCR.

### Quantification of Gene Expression

Total RNA was isolated using TRIzol, treated with DNase I, and then stored at -80° C. cDNA was synthesized using SuperScript™ III First-strand Synthesis SuperMix. Transcript levels were measured by RT-qPCR using Power SYBR Green PCR Master Mix and primers listed in Table 1. To improve quantification of mouse and human TAZ transcripts in murine samples, a TAZ DNA fragment was amplified from mouse cDNA or a plasmid carrying human TAZ using primers indicated in Table 1. The murine or human fragment was serially diluted and amplified using a primer set targeting mouse Taz (Table 1). Amplification efficiency for each species was calculated from plots of Log₁₀(Concentration) vs. Ct.

TAZ protein expression was evaluated using the Wes capillary western blotting system (ProteinSimple). Primary antibody against TAZ (Santa Cruz Bio., Cat # sc-365810) was used to recognize both human and mouse TAZ isoforms.

### Histology and Immunostaining

Mouse samples were collected and fixed overnight in 4% paraformaldehyde at 4° C. For histology, samples were embedded in paraffin and sectioned at 7 µm. Dewaxed samples were fixed in Bouin’s Fixative and then stained with Fast Green/Sirius Red. Images were taken under a brightfield microscope and quantified using ImageJ.

For immunofluorescent staining, fixed samples were cryoprotected in 30% sucrose/PBS overnight at 4° C. 10 µm cryosections were stained using anti-GFP (Rockland inc., Cat# 600-101-215) and anti-TNNI3 (Abcam, Cat# 56357) primary antibodies.

For analyses of apoptosis, paraffin sections were dexaxed, rehydrated, and treated with proteinase K. TUNEL assay was performed using the In Situ Cell Death Detection Kit (Sigma). Sections were then stained with anti-TNNI3 antibody and DAPI and imaged using a Keyence epifluorescent or Olympus FV3000RS confocal microscope.

### RNA in Situ Hybridization

Transcripts of hTAZ and Actn2 were visualized using RNAscope® Multiplex Fluorescent Reagent Kit v2 (Cat# 323100) and RNA probes from Advanced Cell Diagnostics (hTAZ Cat# 828651-C2; Actn2 Cat# 569061). Staining was carried out according to manufacturer’s protocol. Briefly, hearts or skeletal muscle samples were fixed with 4% PFA, embedded in O.C.T. and sectioned at -20° C. Sections were pretreated with protease and incubated with hybridization probes and amplification solutions as directed by the manufacturer’s protocol. After developing in situ signal by incubation with fluorescent dyes, an additional staining with fluorophore-conjugated wheat germ agglutinin (Invitrogen Cat# W32464) was performed to visualize the cell membrane.

### Detection of Apoptotic CMs

After dewaxing and rehydration, paraffin-embedded sections were pretreated with 10 µg/ml proteinase K at room temperature for 15 min. After washing, the TUNEL labeling mixture (In Situ Cell Death Detection Kit; Sigma, Cat# 11684795910) was applied to sections and incubated at 37° C. for one hour. Sections were then washed in PBS and a standard immunofluorescent staining with anti-TNNI3 antibody was performed. Sections were imaged with an epifluorescent microscope (Keyence) or a laser scanning confocal (Olympus FV3000RS).

### Human Myocardial Samples

Deidentified BTHS human heart samples were obtained from the Barth Syndrome Registry or from archival specimens from Boston Children’s Hospital under protocols approved by the Boston Children’s Hospital Institutional Review Board. Control healthy adult samples were obtained from Biochain Institute Inc. (Cat# 50180957).

### Cardiolipin Analysis

CL was extracted into chloroform/methanol and analyzed by Matrix Assisted Laser Desorption/Ionization-Time of Flight Mass Spectrometry(MALDI-TOF MS)²⁸ with minor modifications¹¹.

### Blood Cell Count

Automated complete blood count was performed using Drew Scientific Hemavet 950FS Hematology Analyzer (Cat# HV950FS). Mice were euthanized with CO₂ and blood samples were collected from the heart and stored in K2EDTA spray coated tubes until analysis.

### Electron Microscopy

Sample were fixed in EM fixative (2.5% Glutaraldehyde 2.5% Paraformaldehyde in 0.1 M sodium cacodylate buffer, pH 7.4) overnight at 4° C. After fixation, tissue is washed in 0.1 M cacodylate buffer and postfixed with 1% Osmium tetroxide (OsO₄)/1.5% Potassium ferrocyanide (KFeCN₆) for 1 hour, washed in water for 3 times and incubated in 1% aqueous uranyl acetate for 1 hr followed by 2 washes in water and subsequent dehydration in grades of alcohol (10 min each; 50%, 70%, 90%, 2x10 min 100%). Subsequently, tissue samples were incubated in propyleneoxide for 1 hr and infiltrated ON in a 1:1 mixture of propyleneoxide and TAAB Epon (Marivac Canada Inc. St. Laurent, Canada). On the next day, samples were embedded in TAAB Epon and polymerized at 60° C. for additional 2 days.

Ultrathin sections (about 60 nm) were cut on a Reichert Ultracut-S microtome, picked up on to copper grids stained with lead citrate and examined in a JEOL 1200EX Transmission electron microscope. Images were recorded with an AMT 2k CCD camera.

### Behavior Studies

To study the locomotor function and fatigue phenotype of TAZ-KO mice, open field assay of spontaneous activity was performed before and after mild treadmill exercise. To further evaluate the exercise capacity of BTHS mice, an exhaustion assay was performed according to previous studies^{29,30}.

First, spontaneous activity was measured after sub-maximal exercise. Mice were individually placed in open field chambers to allow free exploration for 6 minutes. These chambers are equipped with monitors and software that measure spontaneous mouse activity (movement, distance traveled, rearing behavior, resting time, as well as time spent at center vs. peripheral of the chamber; Ethovision XT 9.0, Noldus, Netherlands). After a baseline recording of resting motor activity, mice were put on a treadmill and acclimated by setting the treadmill at 3 m/min for 1 minute. Then the treadmill was set to 3 m/min and increased to 8 m/min over 4 minutes. The 8 m/min pace was maintained for an additional 10 minutes. Immediately after the exercise, mice were placed back in their original open field chamber and their spontaneous activity was again monitored for 6 minutes.

Second, the maximal exercise capacity of mice was measured using an exhaustion assay, as described previously^{29,30}. The treadmill is equipped with an electrified metal grid at the end of the moving belt to provide motivation for mice to run rather than rest on the grid. Animals were first trained to use treadmill and then run at 10 m/min for a total of 14 minutes. Mice were removed from the treadmill for exhaustion if 1) they stayed on the shock grid for over 5 seconds and won’t get back to running or 2) the third time willing to sustain 2 sec or more of electric shocking rather than return to the treadmill. The time mice spent on treadmill running was recorded and the shorter running time reflects more severe exercise intolerance.

### Statistics

Data are presented as mean ± standard deviation. Student’s t-test was used for two samples or ANOVA followed by Tukey’s post-hoc test for three or more samples. Statistical testing was performed using GraphPad Prism 8.

### Results

### Global Deletion of Tafazzin in Mice Caused Embryonic and Neonatal Lethality

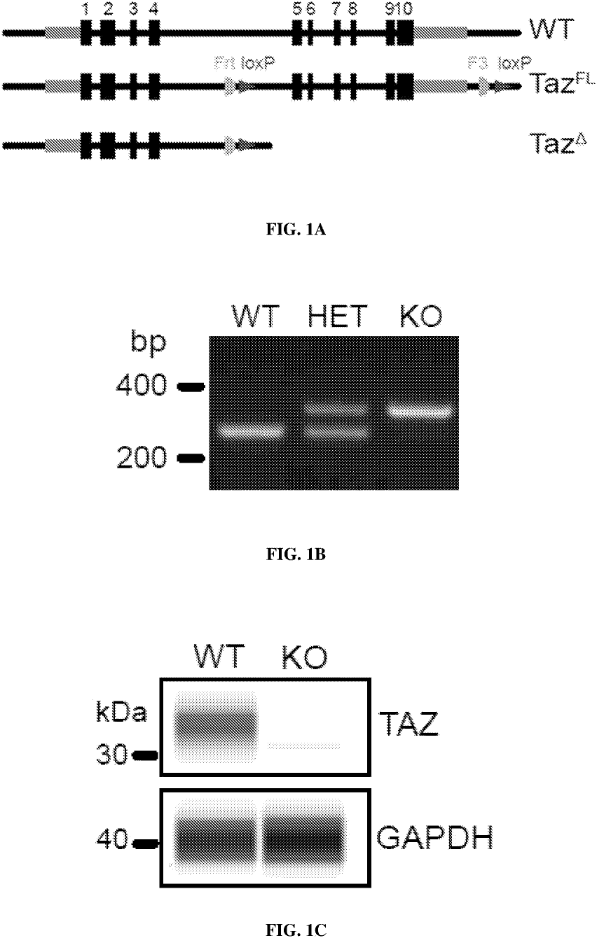

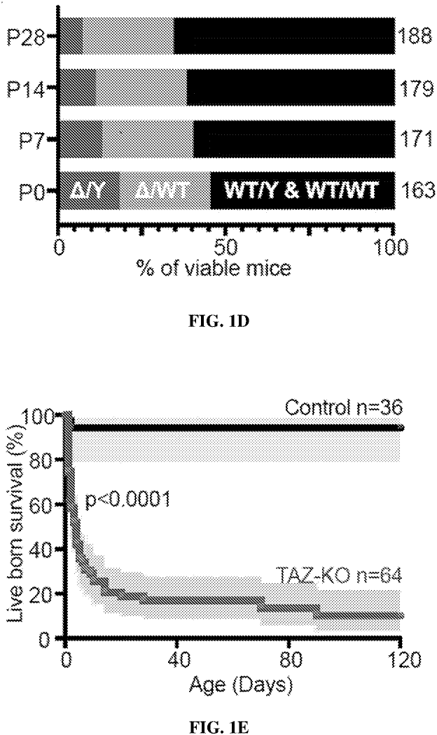

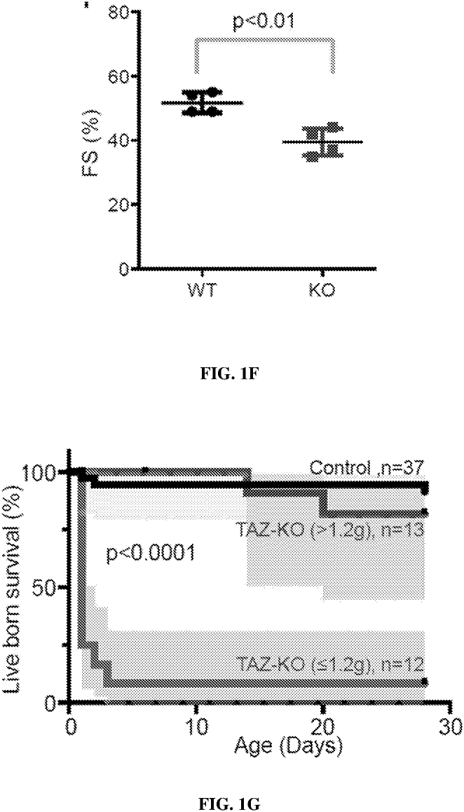

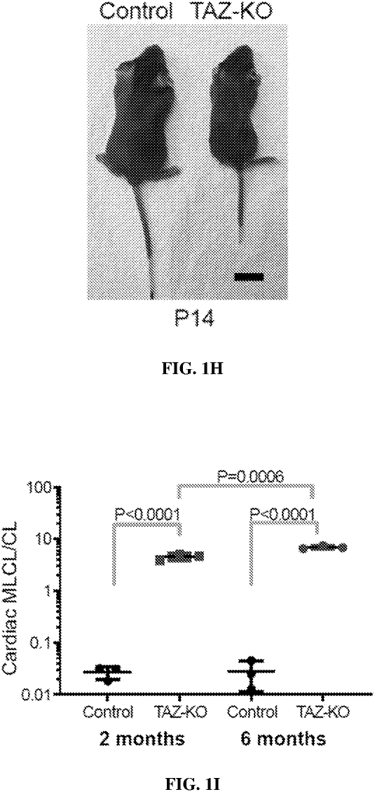

The Taz^{Δ} allele was generated by treating Taz^{fl} sperm with Cre (FIG. 1A)²⁵. Since male mice lacking Taz are sterile³¹, heterozygous females (Taz^{+/Δ}) were used to generate Taz^{Δ/Y} constitutive knockout (TAZ-KO) and littermate control (Taz^{+/Y}) offspring. The mutant and wild-type alleles were easily distinguished by PCR genotyping (FIG. 1B). Effective TAZ deletion in TAZ-KO mice was confirmed by immunoblotting using a capillary western system (FIG. 1C). TAZ-KO mice were born at below the expected Mendelian ratio (FIG. 1D), although this was not statistically significant due to the relatively small sample size. Most liveborn TAZ-KO died in the neonatal period, so that only -20% of live born mice survived to term (FIG. 1E). TAZ-KO neonates had mild to moderate ventricular systolic dysfunction (FIG. 1F).

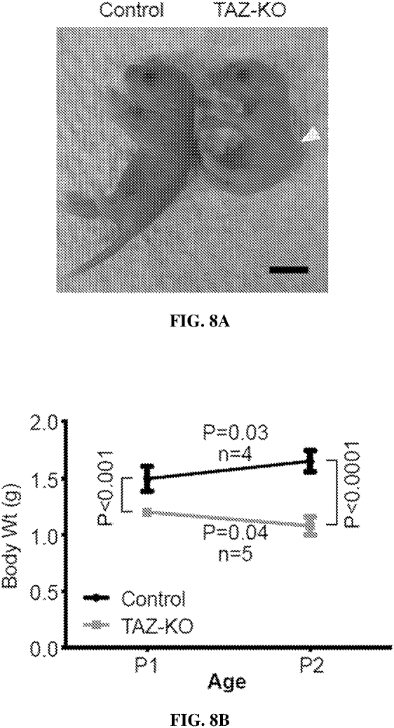

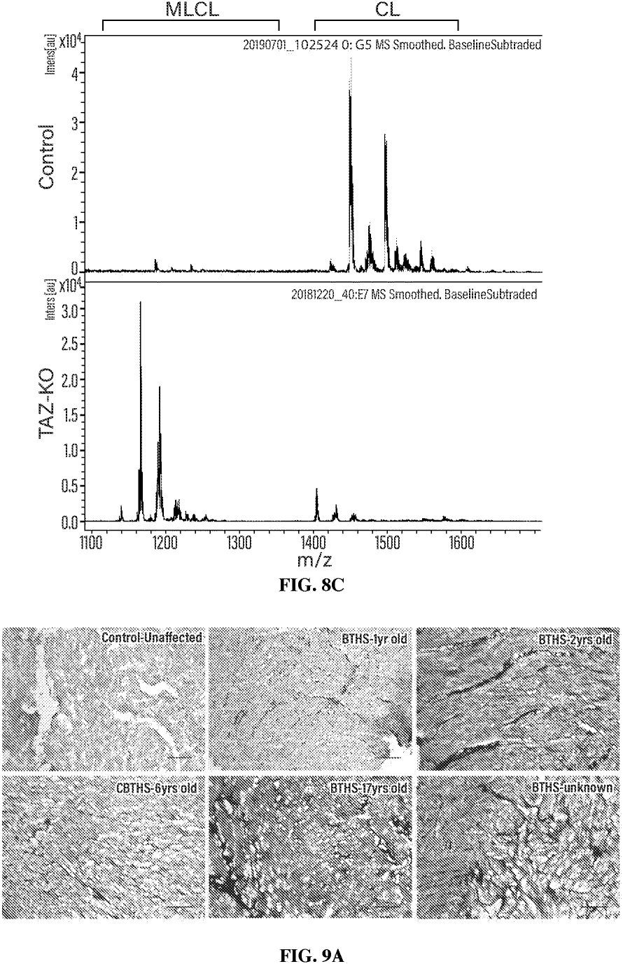

Like BTHS patients^{1,2}, TAZ-KO newborn mice exhibited growth retardation, poor feeding, and muscle weakness. TAZ-KO newborns had reduced movement, hunchback, and dropping forelimbs, a sign of neuromuscular weakness³² (FIG. 8A). A milk spot was rarely observed in TAZ-KO neonates, and body weight declined between P1 and P2 in TAZ-KO whereas it increased in control littermates (FIG. 8B). Neonatal survival was related to birth weight, as TAZ-KO mice with body weight greater than 1.2 g at postnatal day 1 (P1) survived better than those with lower body weight (FIG. 1G). TAZ-KO mice who survived the newborn period had lower body weight and reduced body length throughout life (FIG. 1H).

TAZ is required for CL remodeling, and BTHS patients have abnormal CL profiles with elevated MLCL:CL ratio^{12,13}. MALDI-TOF mass spectrometry of lipids isolated from TAZ-KO hearts was confirmed and the MLCL:CL ratio was markedly elevated in TAZ-KO (FIG. 1I and FIG. 8C).

Together, these data demonstrate that Taz is essential for embryonic and neonatal development and survival, and indicate that TAZ-KO mice recapitulate many of the clinical and biochemical hallmarks of BTHS patients.

### Cardiac Phenotype of TAZ-KO Mice

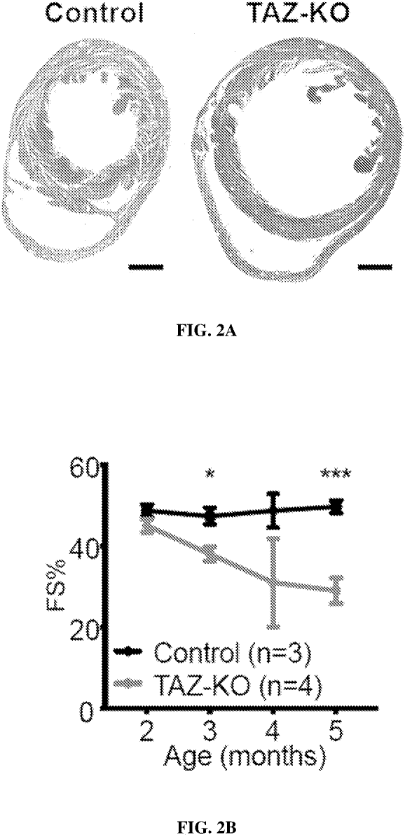

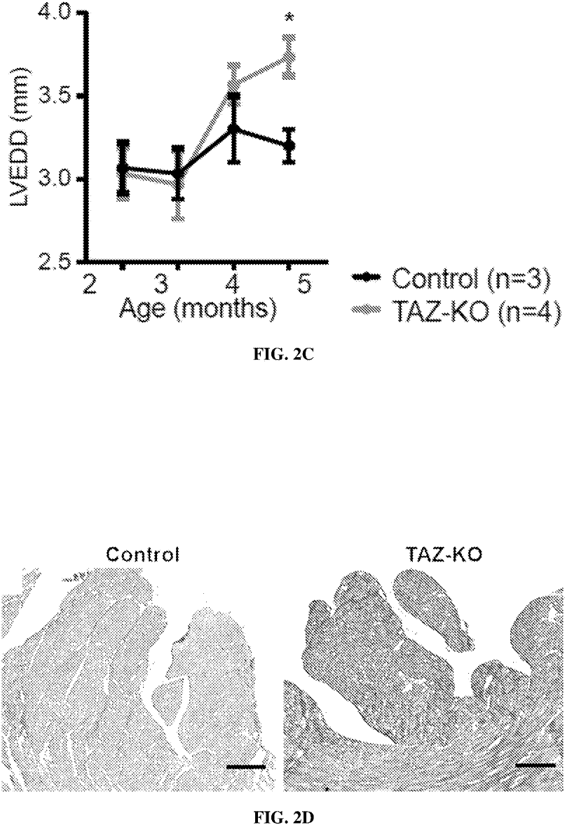

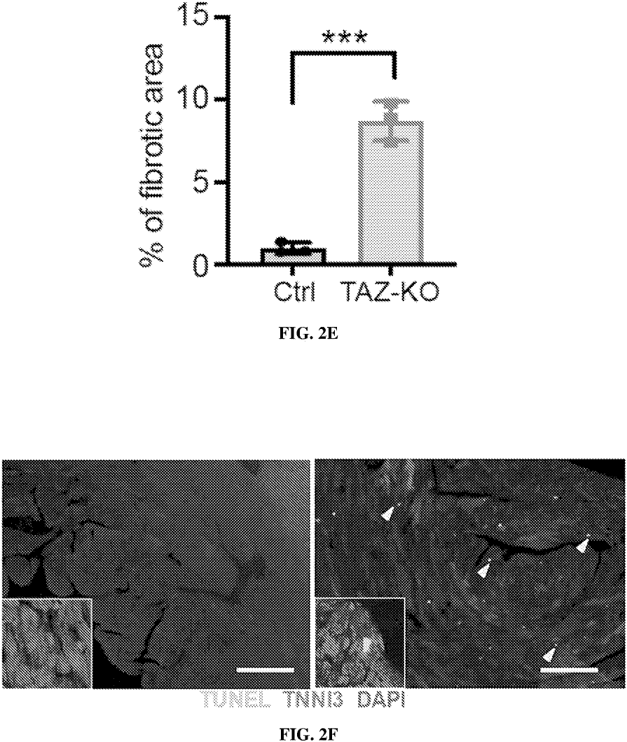

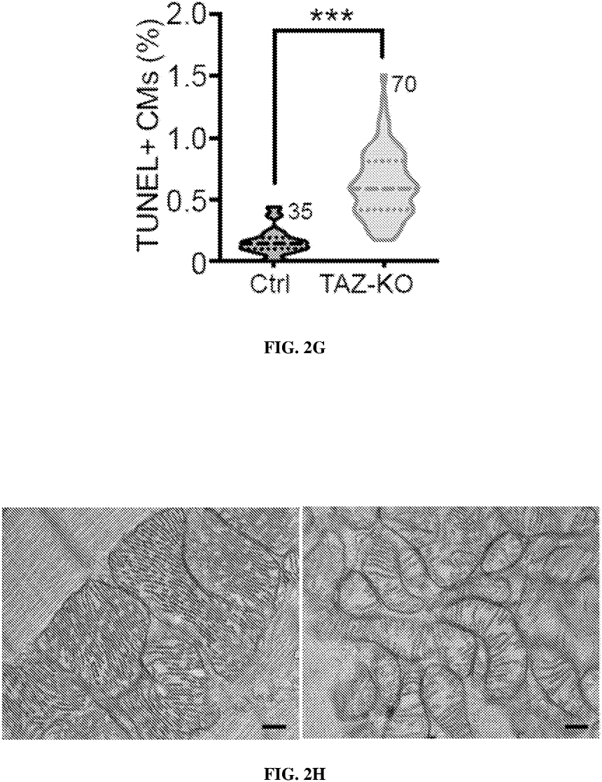

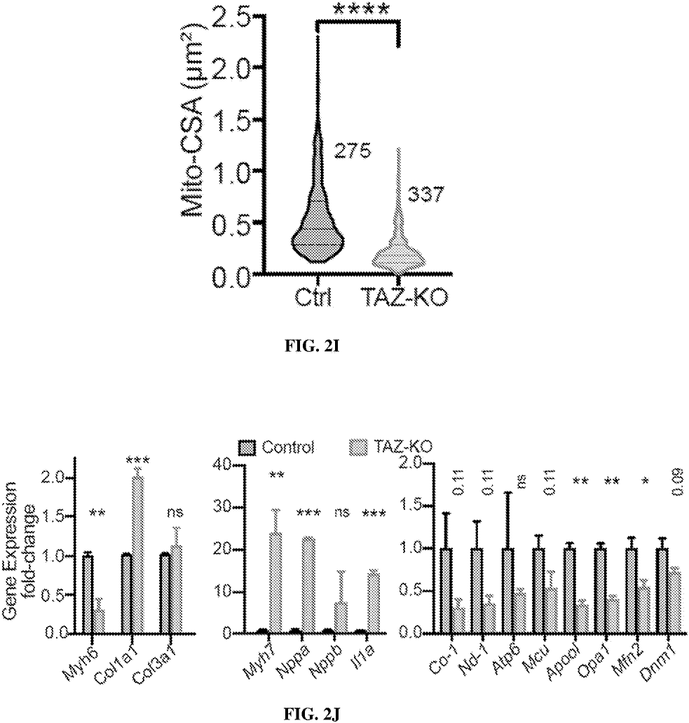

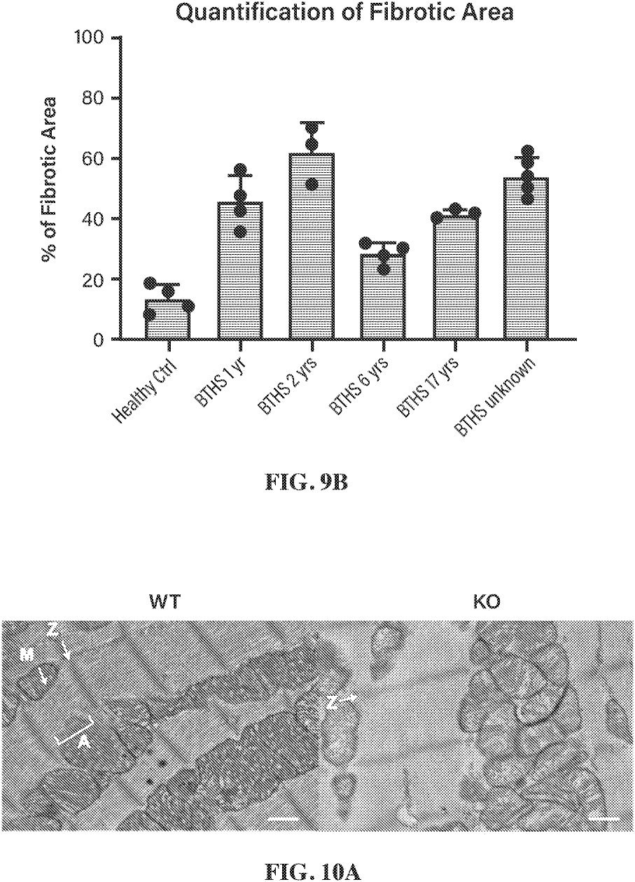

Although most TAZ-KO mice did not survive the neonatal period, through intensive breeding, sufficient TAZ-KO mice was obtained for analysis of adult phenotypes. In these TAZ-KO survivors, which largely had initial body weight > 1.2 g at P1, the left ventricle was dilated and thin-walled compared to control, consistent with a dilated cardiomyopathy phenotype (FIG. 2A). Although the neonatal studies showed cardiac dysfunction (FIG. 1F), at 4-8 weeks-of-age TAZ-KO survivors had relatively normal cardiac function (FIG. 2B). This difference likely reflects survival bias. In the TAZ-KO survivor cohort, cardiac dysfunction typically manifested after 8 weeks-of-age and became progressively more severe over the subsequent 3 months (FIGS. 2B to 2C). Adult TAZ-KO hearts had significantly increased fibrosis (FIGS. 2D to 2E). it was confirmed that human BTHS hearts also have considerable cardiac fibrosis (FIG. 9). Because cardiac fibrosis often occurs in the setting of increased CM death, CM apoptosis was measured in TAZ-KO and littermate control hearts. TUNEL staining demonstrated significantly increased CM apoptosis in TAZ-KO (FIGS. 2F to 2G).

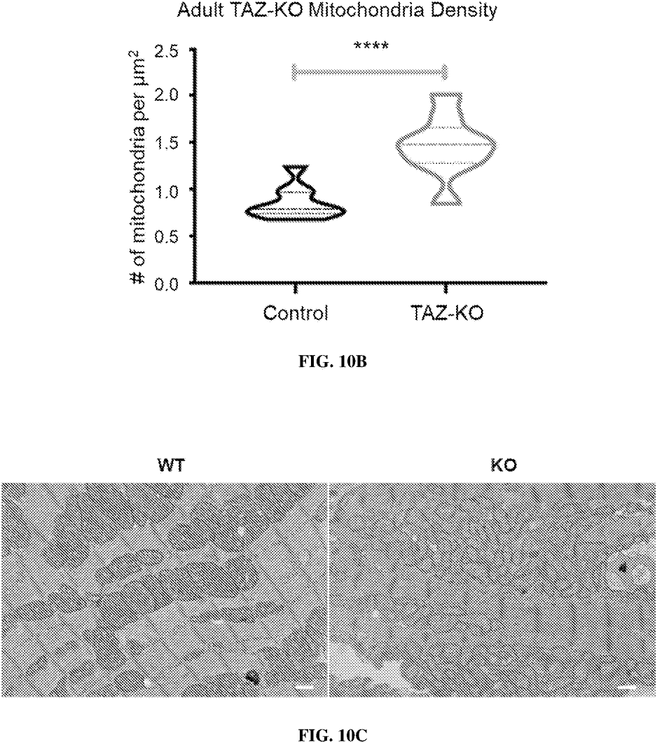

Electron microscopy was used to evaluate the ultrastructure of TAZ-KO cardiomyocytes and mitochondria. Overall, TAZ-KO sarcomere Z-lines had normal morphology, but the A bands and M lines were poorly delineated, suggesting abnormal sarcomere structure in the absence of Taz (FIG. 10A). A drastic reduction in the internal complexity of mitochondria was observed in mutant hearts (FIG. 2H). Whereas the cristae in control mitochondria were neatly organized and packed in parallel, mutant mitochondria had reduced inner mitochondrial membrane content and cristae density. Compared to controls, TAZ-KO mitochondria were smaller (FIG. 2I), and as a result there was a greater number of mitochondria per unit area (FIG. 10B). The spatial organization of mitochondria was also impaired by TAZ deficiency: whereas control mitochondria were aligned adjacent to sarcomeres, TAZ-KO mitochondria formed poorly organized clusters (FIG. 10C).

TAZ-KO and control ventricular RNA was analyzed for expression of genes related to cardiac stress, inflammation and fibrosis, and mitochondria (FIG. 2J). Consistent with myocardial stress, in TAZ-KO Myh6 was downregulated, and Myh7 and Nppa were markedly upregulated. The inflammatory cytokine Il1a was also strongly upregulated. Collagen type I (Col1a1) was significantly upregulated whereas the expression of collagen type III (Col3a1) was unchanged. Nuclear mitochondrial transcripts were significantly downregulated (Apool, Opal, Mfn2) or had a tendency to downregulation (Mcu, Dnm1). Mitochondrially encoded transcripts Co-1 and Nd-1 also tended to be downregulated.

### Skeletal Muscle Phenotype and Neutrophil Counts of TAZ-KO Mice

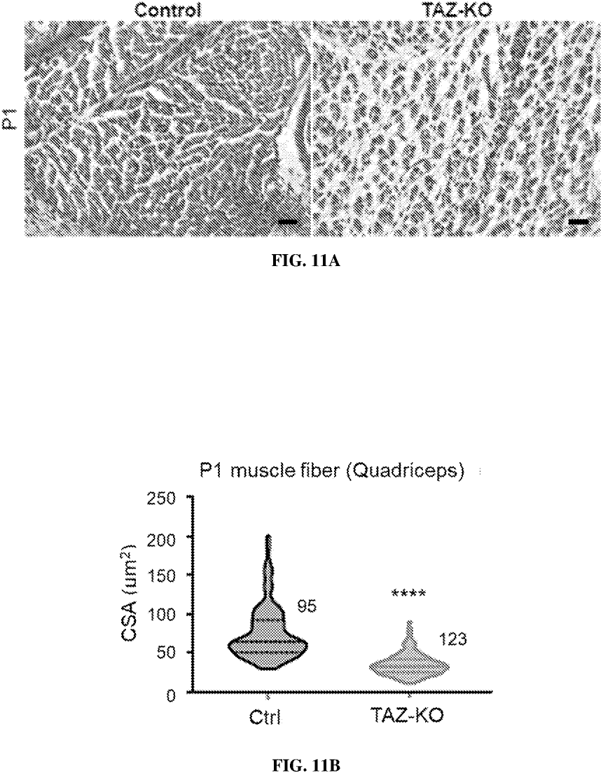

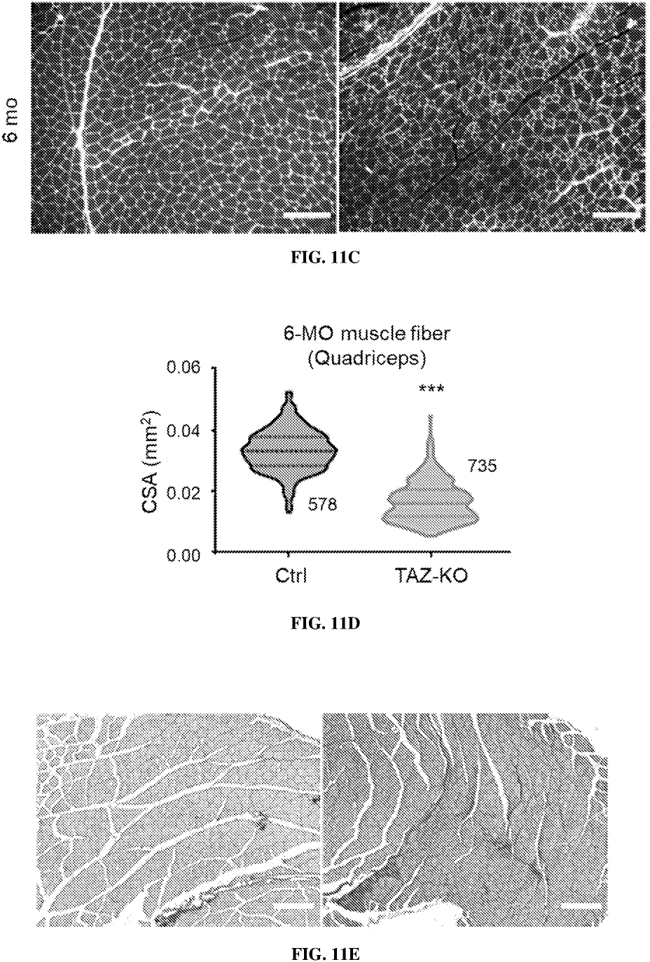

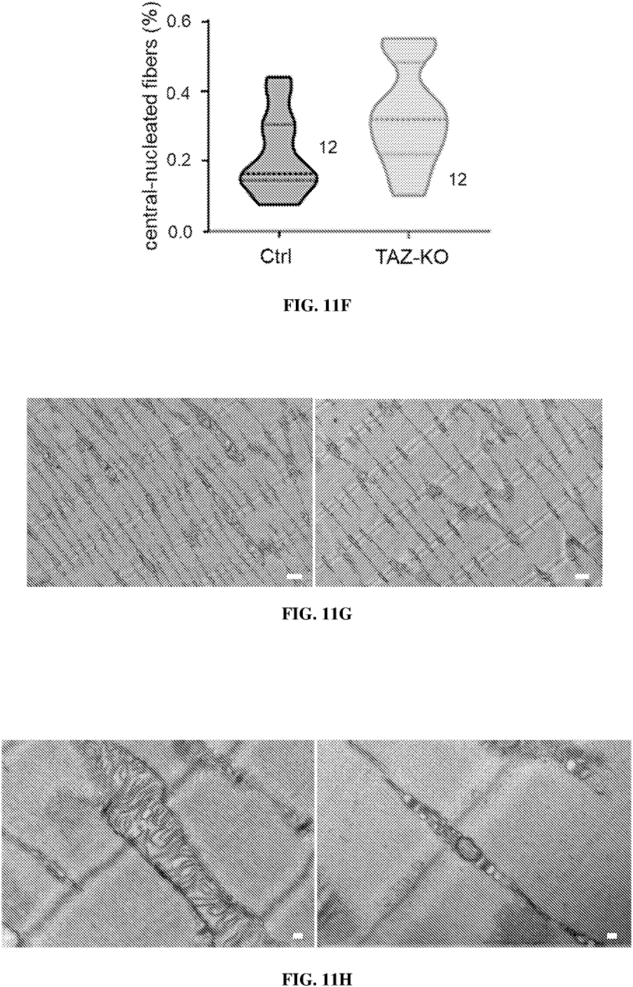

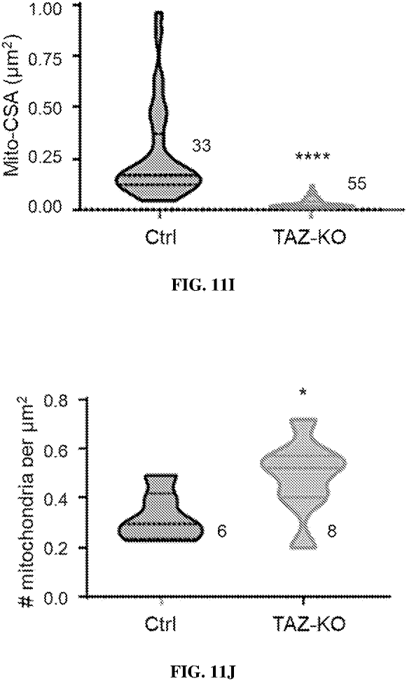

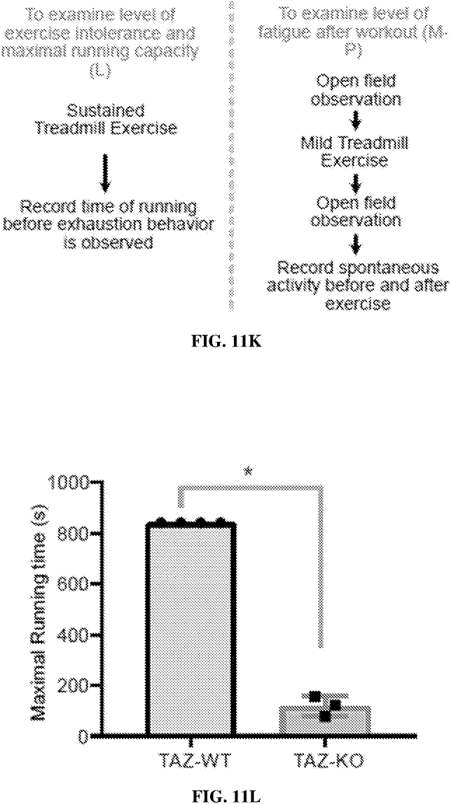

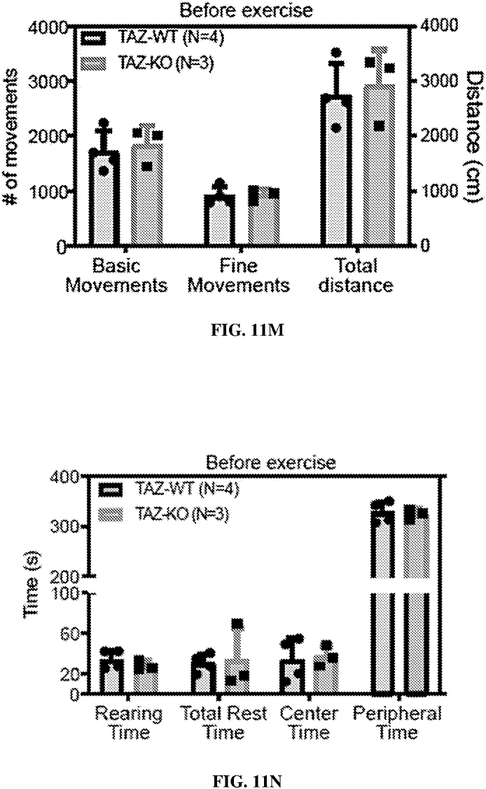

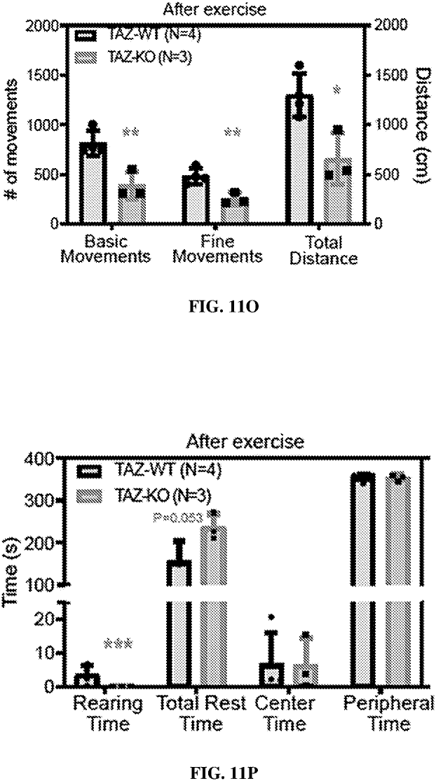

Since skeletal muscle weakness/fatigability is a cardinal feature of BTHS³, and mice exhibited signs of muscle weakness, the TAZ-KO skeletal muscle phenotype was examined. Newborn TAZ-KO mice had smaller caliber quadriceps muscle fibers compared to control littermates, and this finding persisted in 6 month-old adult TAZ-KO survivors (FIGS. 11A to 11D). The adult TAZ-KO mice also exhibited mild skeletal muscle fibrosis (FIG. 11E). Skeletal myopathy sometimes triggers potent muscle fiber regeneration, which results in muscle fibers with centrally located nuclei. However, a significant change was not observed in this parameter in TAZ-KO (FIG. 11F).

Mitochondrial ultrastructure in skeletal muscles was analyzed by electron microscopy (FIGS. 11G to 11H). Consistent with observations in the heart, TAZ-KO skeletal muscle had severely compromised cristae, markedly reduced mitochondrial cross-sectional area (FIG. 11I), and increased number of mitochondria per unit area (FIG. 11J). To measure the impact of these abnormalities on skeletal muscle function, two tests of skeletal muscle performance was performed (FIG. 11K). First, to evaluate the level of exercise intolerance, TAZ-KO and WT mice ran on a treadmill at 10 m/min. Mice were closely monitored during the trial and immediately removed from the treadmill once they exhibited signs of exhaustion by excessively resting on a shock grid positioned behind the treadmill (see Detailed Methods). At 6 months-old, TAZ-KO mice ran for an average of 110 seconds, whereas wild-type mice did not show exhaustion by the end of the test (840 seconds; P<0.05; FIG. 11L), indicating severely compromised exercise capacity.

Next, it was sought out to simulate the fatigue that BTHS patients experience after even low levels of exertion. Animals were first place individually in open field chambers, equipped to trace and measure the movement of mice within the chamber. After baseline measurements for 6 minutes, mice were acclimated to a treadmill and run at a lower speed for 14 minutes. Immediately after exercise, mice were immediately placed in their original open field chamber and their activity was again recorded for 6 minutes. As shown in FIGS. 11M to 11N, TAZ-KO mice had normal baseline locomotor activity prior to exercise. After running on the treadmill, TAZ-KO had significantly less basic as well as fine movements, and traveled less overall distance compared to WT controls (FIG. 11O). Furthermore, TAZ-KOs rarely reared on their hindlimbs after exercise and tended to spend more time resting compared to WTs (FIG. 11P). The time spent in the field center compared to the periphery, a measure of anxiety, was comparable between TAZ-KO and WT groups.

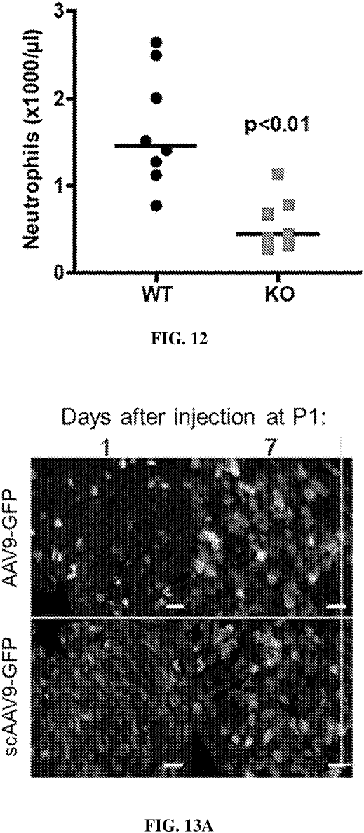

Since neutropenia is another clinical feature frequently observed on BTHS patients, the levels of circulating neutrophils in 6-month-old TAZ-KO and WT mice were examined. TAZ-KO mice have significantly lower circulating neutrophil concentration than WT controls (FIG. 12). However, the number of neutrophils in TAZ-KO was still within the normal range for mice (0.1-2.4 K/µL), according to the manufacturer’s CBC Parameter Guide (FIG. 12). Neutropenia in BTHS patients can be intermittent and sporadic, so more extensive studies are required to fully evaluate this phenotype in TAZ-KO mice.

### Cardiomyocyte-Restricted TAZ Deletion

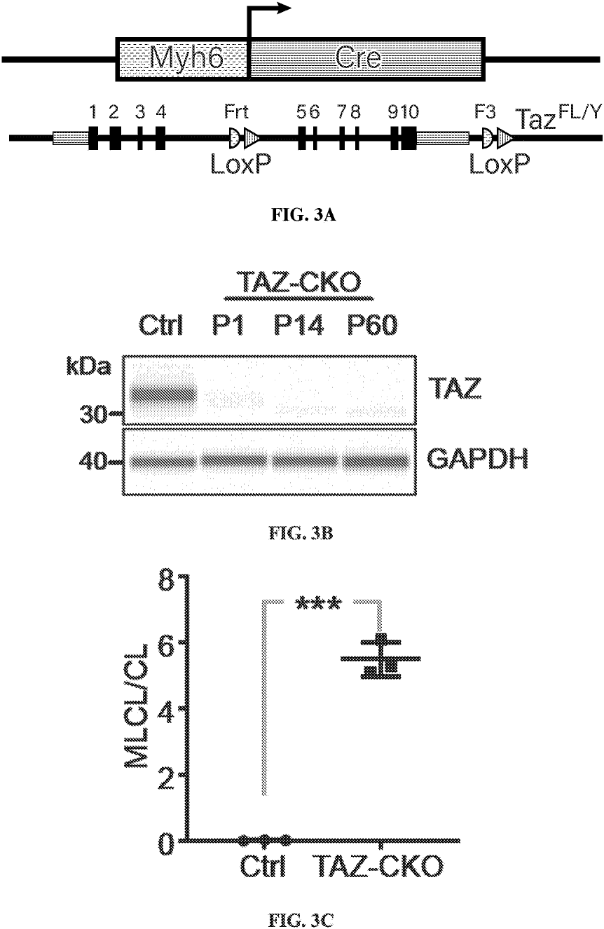

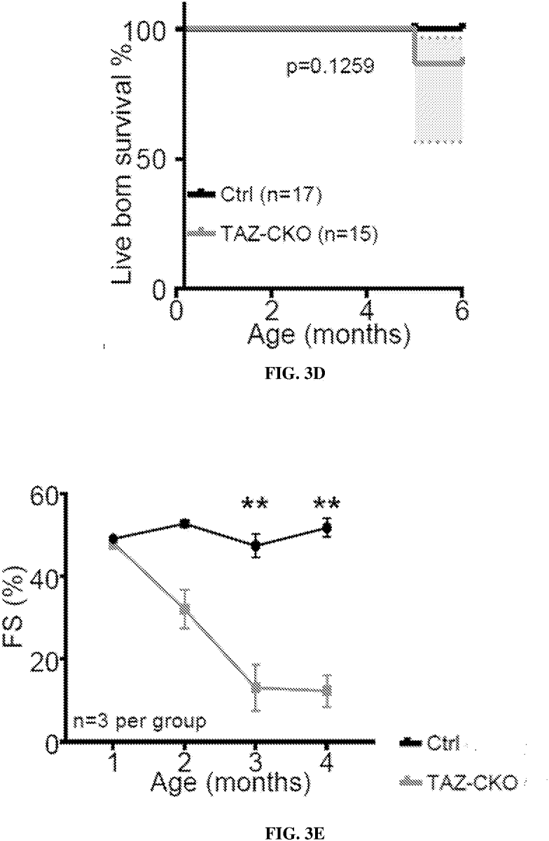

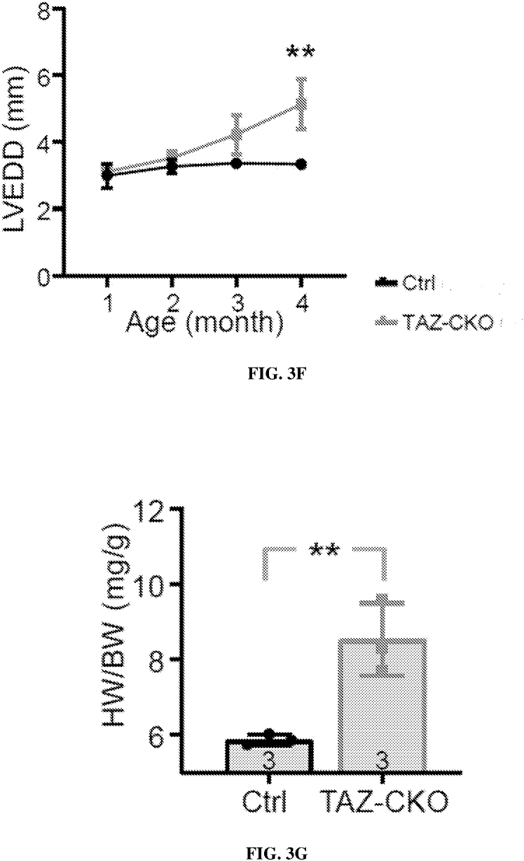

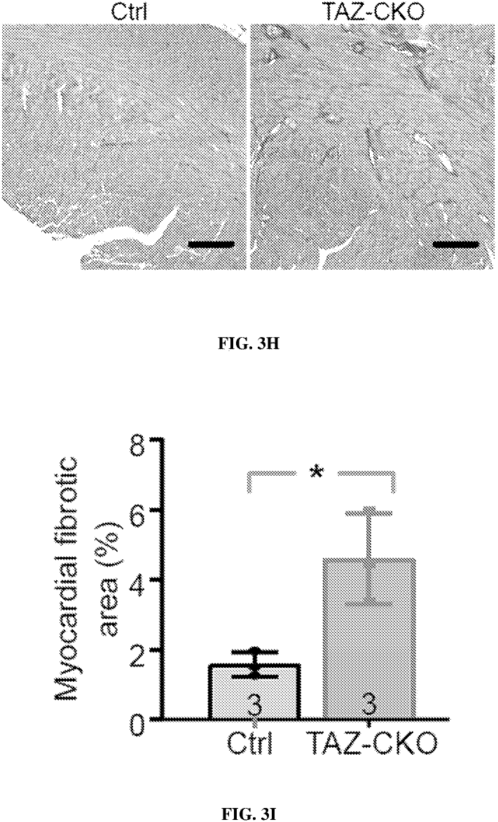

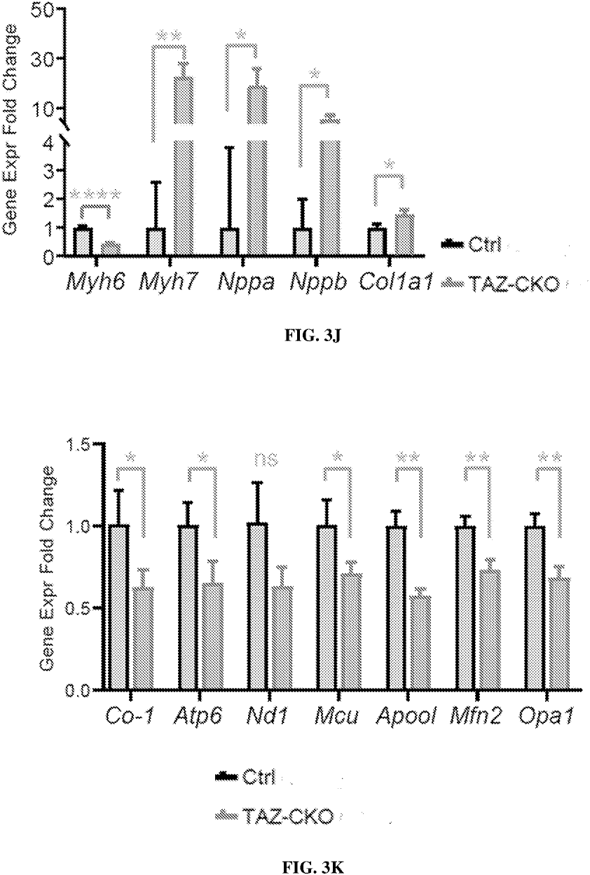

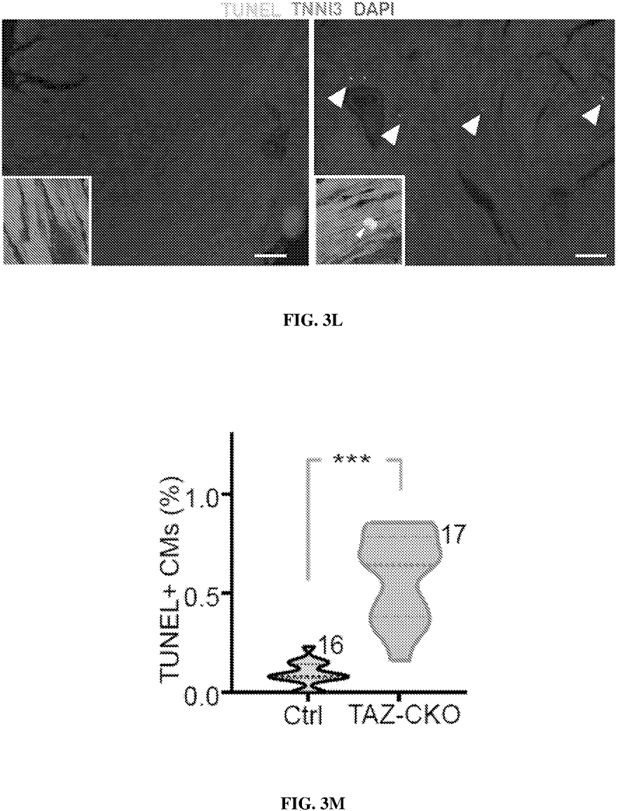

To assess the contribution of Taz inactivation in cardiomyocytes to the TAZ-KO phenotype, cardiomyocyte-specific Cre (Myh6-Cre) were used to inactivate the Taz^{fl} allele (FIG. 3A). The cardiomyocyte-specific Taz mutant mice (TAZ-CKO; Taz^{fl/Y}; Myh6-Cre) were compared to littermate controls (Taz^{+/Y}; Myh6-Cre). The loss of TAZ protein by capillary immunoblotting was validated (FIG. 3B). MALDI-TOF mass spectrometry was also used to confirm that the MLCL/CL ratio was elevated in TAZ-CKO hearts (FIG. 3C).

Next, the perinatal survival and cardiac function of TAZ-CKO mice was monitored. TAZ-CKO mice were born at the expected Mendelian ratio, and their body weight did not significantly differ. The mice survived normally to adulthood, and mice rarely died during 6 months of observation (FIG. 3D). TAZ-CKO had normal LV size and function as neonates (FIGS. 3E to 3F). LV function progressively declined (FIG. 3E). LV dilatation became statistically significant at 4 months (FIG. 3F), and HW/BW ratio was elevated when examined at 6 months (FIG. 3G). Histological evaluation demonstrated myocardial fibrosis and CM apoptosis in TAZ-CKO (FIGS. 3H to 3I, FIGS. 3L to 3M). Consistent with reduced cardiac function in TAZ-CKO hearts, genes associated with cardiac stress were significantly upregulated, whereas genes that are critical for mitochondrial function and morphology were found to be suppressed (FIGS. 3J to 3K).

Collectively, the data show that the TAZ-CKO model does not reproduce the fetal and perinatal loss observed in the whole body TAZ-KO, perhaps due to fetal or perinatal requirement of TAZ in non-cardiomyocytes, e.g. skeletal muscle. On the other hand, TAZ inactivation in CMs was sufficient to reproduce the progressive dilated cardiomyopathy and cardiac fibrosis observed in adult mice.

### AAV-hTAZ Rescue of TAZ-KO Neonatal Lethality

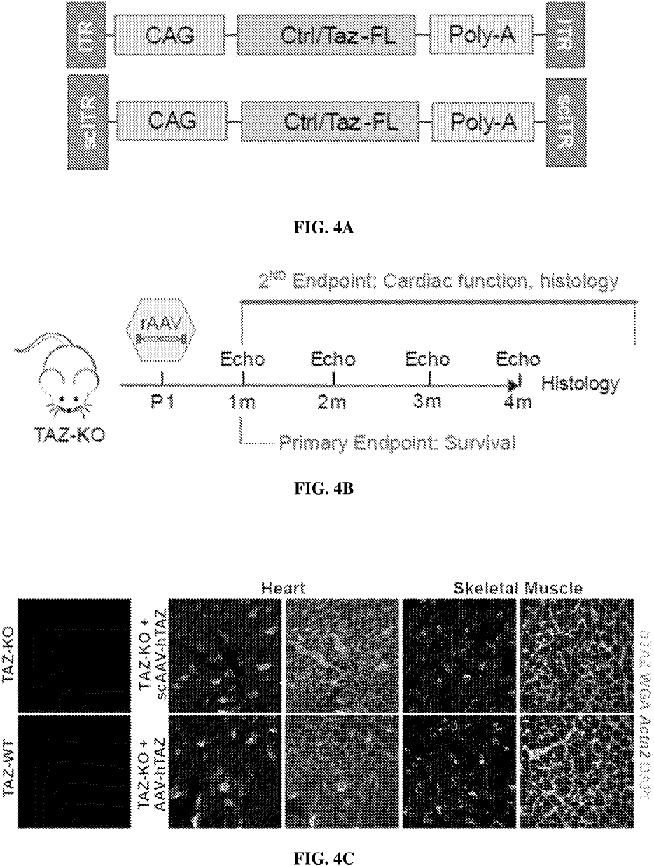

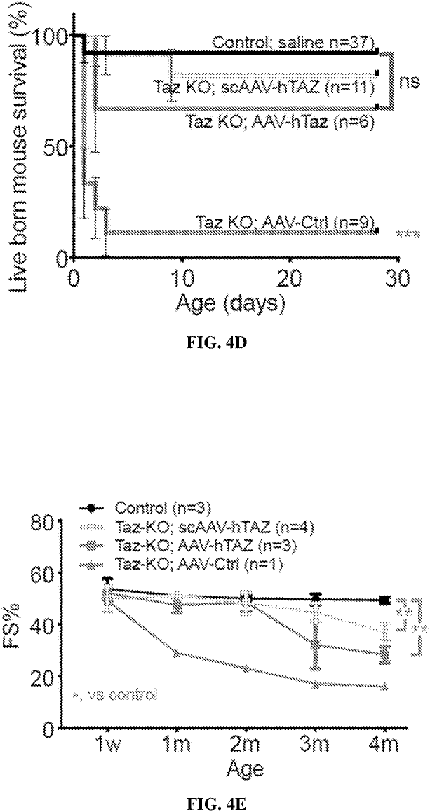

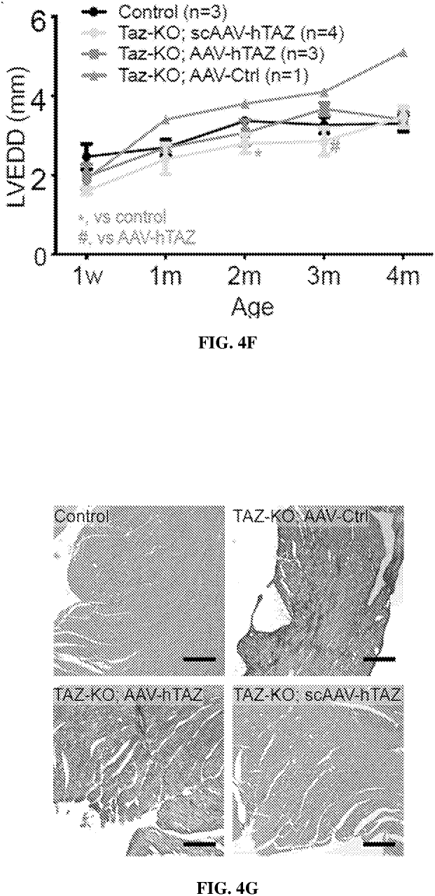

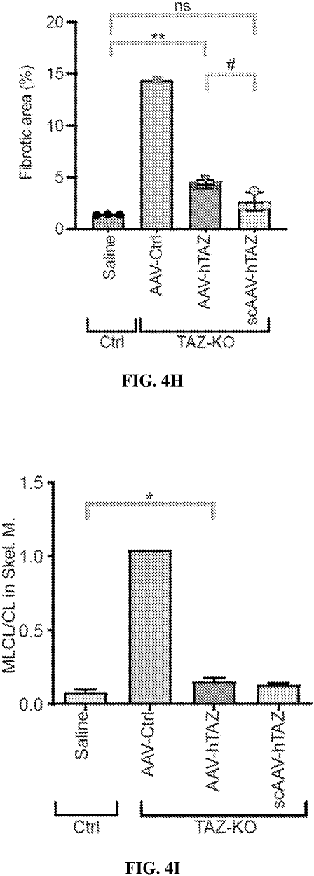

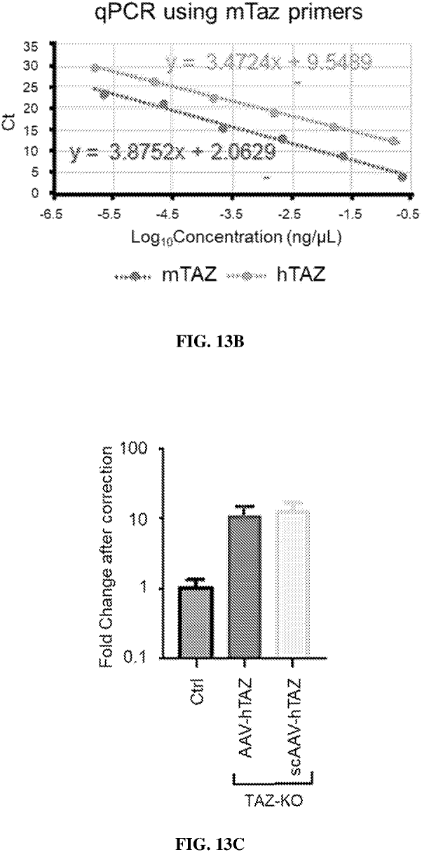

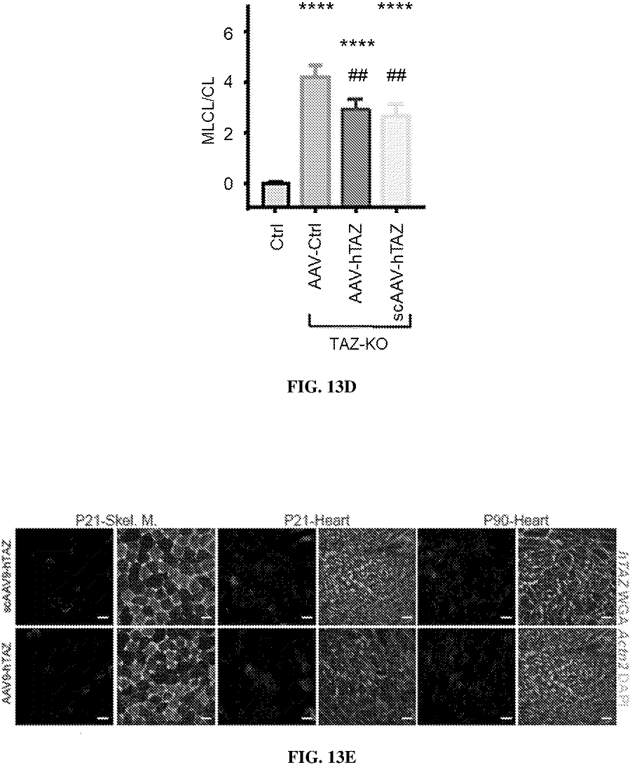

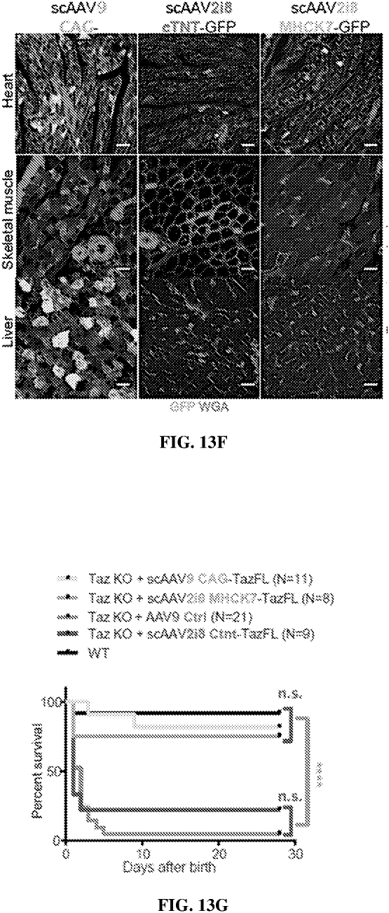

AAV gene therapy is an attractive strategy to treat Barth syndrome. AAV9 was generated in which full length human TAZ was expressed from the potent CAG promoter (AAV-hTAZ; FIG. 4A). Both AAV9 and CAG are components of the FDA-approved gene therapy Zolgensma. AAV carrying the coding sequence of luciferase (AAV-Ctrl) was used as the control virus. First, the impact of AAV-hTAZ was studied on the demise of 92% of low body weight (< 1.2 g at P1) TAZ-KO mice in the first week of life. The experiment timeline is summarized in FIG. 4B. At birth, TAZ-KO pups were weighed and genotyped. At P1, low body weight TAZ-KO or control littermates were treated with either AAV-hTAZ or AAV-Ctrl via subcutaneous injection at a dose sufficient to transduced 65% of cardiac and 60% of skeletal muscle cells, as measured at P7 by RNA in situ hybridization using an RNA probe specific to the codon optimized hTAZ transcript (FIG. 4C) or by administration of a similar dose of AAV-GFP (FIG. 13A). The primary endpoint was survival to P28. Secondary endpoints were cardiac function, as assessed by echocardiography monthly for 4 months and cardiac fibrosis at 4 months. Conversion of the single stranded AAV genome to a double-stranded episome is a rate-limiting step of AAV transduction³³, and this process can be expedited by self-complementary AAV³⁴ (scAAV; FIG. 13A). Because the rapid neonatal loss of TAZ-KO mice made transduction kinetics a potential concern, scAAV-hTAZ was also included (FIG. 4A) in the study.

It was sought to estimate the level of AAV-mediated hTAZ expression by qRTPCR. However, hTAZ and mTaz nucleotide sequences differ, so that qPCR primers did not amplify both transcripts with the same efficiency. This problem was circumvented by using standard curves (FIG. 13B) to compare relative expression AAV-expressed hTAZ and endogenous mTAZ (FIG. 13C; see Detailed Methods). AAV-hTAZ and scAAV-hTAZ drove equivalent levels of hTAZ cardiac expression (FIG. 13C), which was estimated to be 10-fold higher than endogenous mTAZ transcript. Consistent with similar hTAZ expression by AAV-hTAZ and scAAV-hTAZ, both vectors partially corrected MLCL:CL to a similar degree (FIG. 13D).

Both AAV-hTAZ and scAAV-hTAZ dramatically enhanced neonatal survival compared to AAV-Ctrl (FIG. 4D). Although survival was slightly higher in scAAV-hTAZ compared to AAV-hTAZ, the difference was not statistically significant at these sample sizes.

Echocardiography was used to monitor the heart function of rescued mice to 4 months-of-age. Unfortunately, due to the high frequency of neonatal demise among low birth weight TAZ-KO, one AAV-Ctrl treated low body weight TAZ-KO mouse was only analyzed, and this mouse exhibited progressive dilated cardiomyopathy that was more severe than observed in unselected TAZ-KO mice (which are almost all in the high body weight category; FIG. 1G and FIG. 2B) . Both AAV-hTAZ and scAAV-hTAZ treated mice were protected against cardiac dysfunction for 2 months (FIG. 4E). At later time points, both hTAZ-treated groups began to show declining cardiac function, which became significantly depressed in the scAAV-hTAZ and AAV-hTAZ groups at 4 months-of-age. However, neither treatment group developed consistent LV dilatation during the 4 month study period (FIG. 4F).

At 4 months-of-age, the cardiac fibrosis was evaluated. Control-treated TAZ-KO heart had extensive fibrosis. In contrast, both hTAZ-treated groups had substantially reduced fibrosis (FIGS. 4G to 4H). scAAV-hTAZ prevented fibrosis to a level that was similar to WT. AAV-hTAZ also markedly reduced fibrosis, although it remained significantly elevated compared to WT. Histological analysis suggested that declining function at later time points was possibly due to insufficient CM transduction: whereas the dose used appeared to transduce 65% CMs at P7, at 21 days and 90 days after the initial treatment only 24% cardiac cells retain transgene expression (FIG. 13E). Similar to the loss of transgene expression in the growing heart, viral genome was similarly greatly reduced in skeletal muscle cells examined 21 days after injection, which could be related to the cell proliferation and viral genome dilution in muscle^{35,36} (FIG. 13E).

As shown earlier, TAZ-CKO mice display normal perinatal survival, which suggests that the BTHS-related mortality in mice is likely non-cardiac and therefore the rescue of TAZ-KO neonatal death by AAV-hTAZ is achieved through TAZ expression in organs besides the heart. The most obvious phenotypic difference between the viable and the poorly surviving TAZ-KO pups was the body weight and the level of gross motor activity. In addition, dead TAZ-KOs were frequently found to have an empty stomach and no milk spot. Therefore, it was hypothesized that the skeletal muscle weakness and failure to compete with stronger littermates for nutrition are important contributors to neonatal death in TAZ-KO mice and that the mechanism of AAV-hTAZ rescue is through improving cardiolipin metabolism in skeletal muscle.

To test this hypothesis, two additional scAAV vectors were designed to direct hTAZ expression selectively in heart (scAAV2i8 cTNT-hTAZ, where AAV2i8 is an engineered AAV serotype that efficiently transduces heart and skeletal muscle, but detargets liver³⁷, and cTNT is a cardiac specific promoter) or the heart and skeletal muscle (scAAV2i8 MHCK7-hTAZ, where MHCK7 is an engineered promoter with striated muscle specificity³⁸). The tissue specificity and tropism of these two vectors was evaluated using GFP as a reporter (FIG. 13F). Other major organs, including liver, brain, thymus, lungs, spleen, pancreas, gut and diaphragm, were also examined and found to be negative for transduction by either of the newly designed viruses. As it was hypothesized, overexpressing hTAZ in the heart did not significantly improve survival of TAZ-KO mice, whereas expressing hTAZ in both the heart and skeletal muscle by scAAV2i8 MHCK7-hTAZ resembles the therapeutic effect of scAAV9 CAG-hTAZ and rescued TAZ-KO mice from neonatal lethality (FIG. 13G). The ratio of MLCL:CL was also examined and found that scAAV2i8 MHCK7-hTAZ corrected the abnormal skeletal muscle CL profile at P7 (FIG. 4I). Overall, these data demonstrate that hTAZ gene replacement, using either single-stranded or self-complementary AAV, efficiently prevents neonatal death by TAZ replacement in skeletal muscle. AAV-hTAZ also protects cardiac function of TAZ-KO mice for at least 3 months, with relatively low cardiomyocyte transduction perhaps accounting for limited therapeutic durability.

### AAV-hTAZ Prevention of Cardiomyopathy in TAZ-CKO Mice

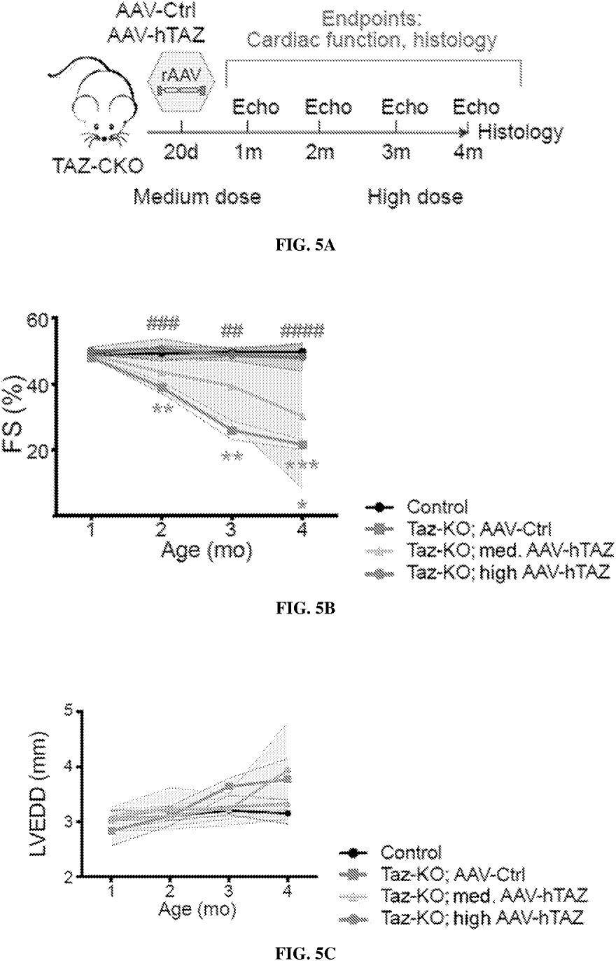

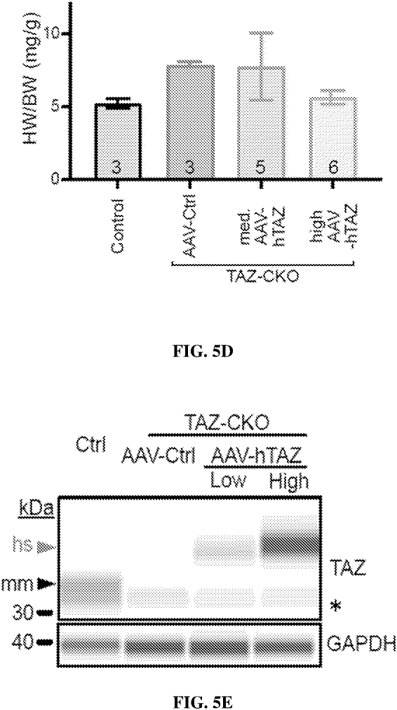

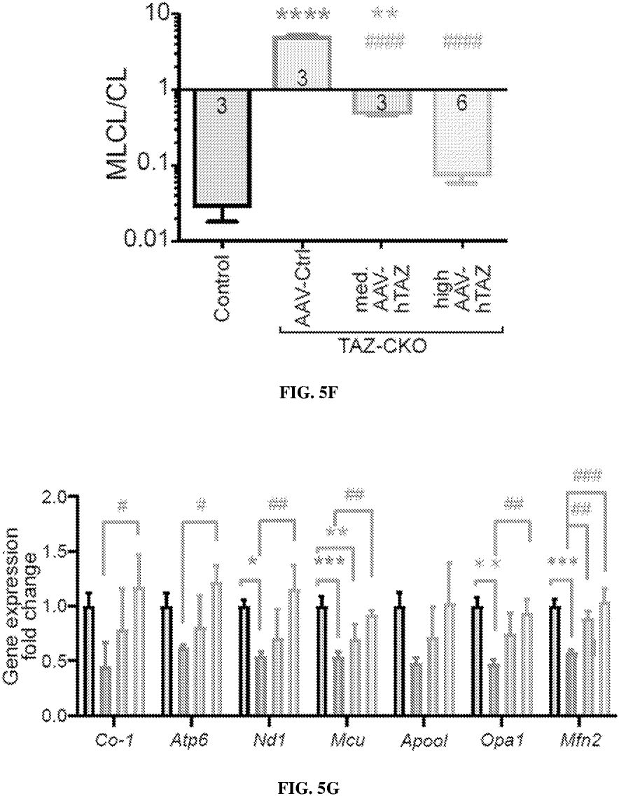

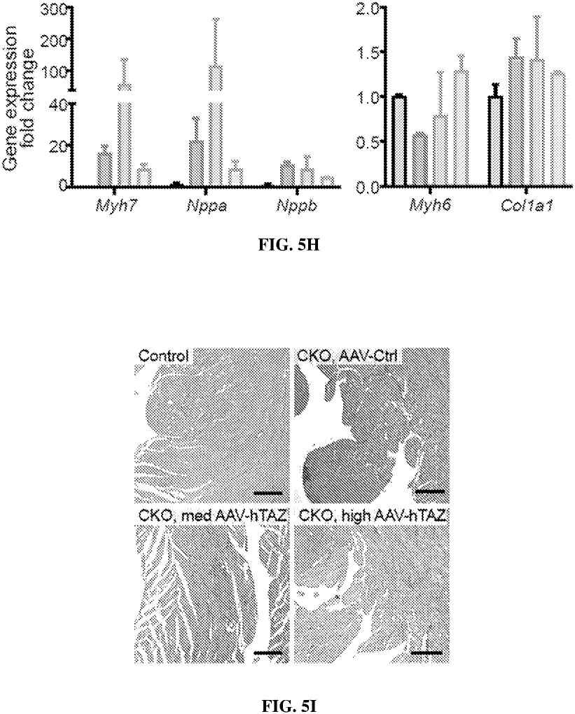

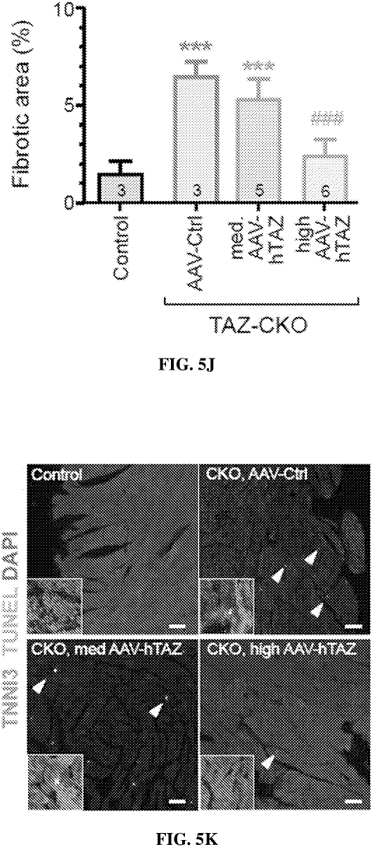

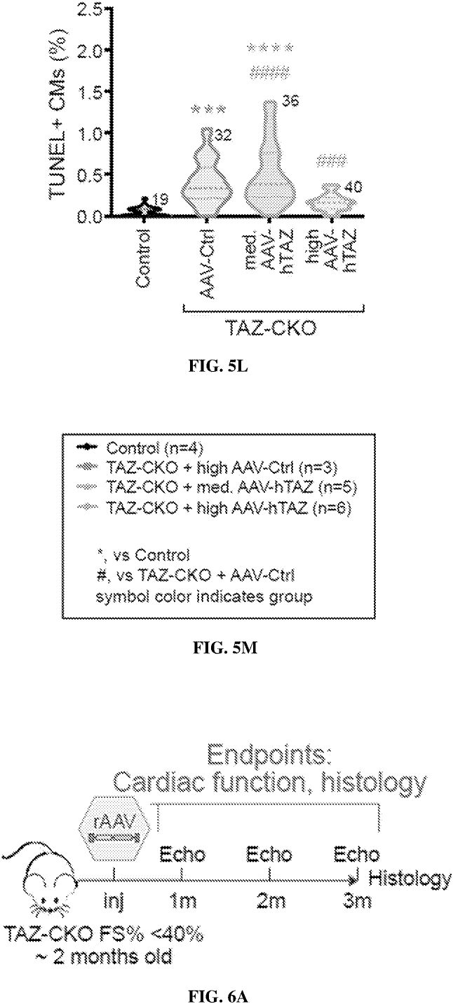

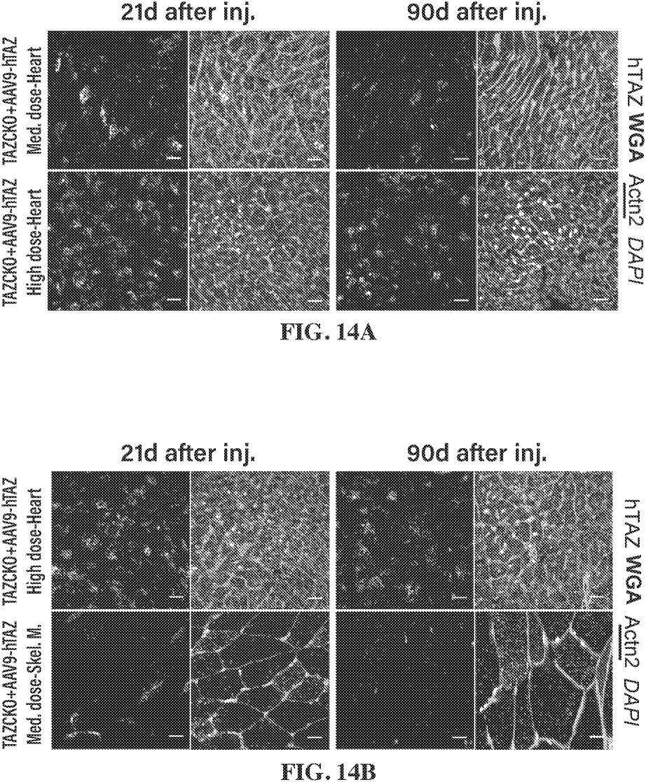

Because the TAZ-CKO model circumvents difficulties with survival in the TAZ-KO model, it is more convenient for assessing therapeutic efficacy and dose response on the cardiomyopathic phenotype. Given that AAV-hTAZ and scAAV-hTAZ had similar efficacy, the AAV-hTAZ was focused on. Similar to the neonatal study, AAV expressing luciferase was used as control virus (AAV-Ctrl). TAZ-CKO mice were treated with AAV-hTAZ or AAV-Ctrl at P20 by intravascular (retro-orbital) injection, prior to the onset of cardiac dysfunction, to determine if gene therapy prevents the development of cardiomyopathy (FIG. 5A). Cardiac function was examined monthly to 4 months of age, when hearts when analyzed for histological endpoints. To evaluate dose-response, medium and high doses of AAV were tested, which transduced -33% and >70% of cardiomyocytes, respectively (FIG. 14A). CM transduction efficiency was similar between 21 and 90 days, indicating that the viral genome was stable in CMs during this period (FIG. 14A).

In AAV-Ctrl treated TAZ-CKO mice, cardiac function became abnormal at 2 months and progressively declined at 3 and 4 months (FIGS. 5B to 5C). High dose AAV-hTAZ provided consistent and durable protection against cardiac dysfunction. In contrast, medium dose AAV-hTAZ resulted in variable results, with a subset of mice exhibiting little improvement in cardiac function compared to control treatment, and other mice exhibiting normalization of cardiac function.

At the end of the study, hearts were collected for molecular and histological analysis. Cardiac hypertrophy seen in TAZ-CKO mice was reduced by high dose AAV-hTAZ (FIG. 5D). This result did not reach statistical significance in ANOVA (p=0.146 with Tukey’s post-hoc test) due to the variable response and large variance in the medium dose group (p=0.0001 when medium dose group was excluded). TAZ expression was measured by capillary western blotting, which distinguished endogenous murine TAZ from hTAZ because hTAZ is larger by virtue of a primate-specific exon. This assay confirmed loss of endogenous TAZ in TAZ-CKO hearts and dose-dependent expression of hTAZ by AAV-hTAZ (FIG. 5E). AAV-hTAZ also normalized the MLCL/CL ratio in a dose-dependent manner, with the medium dose reducing it to an intermediate level, and the high dose making it comparable to control mice (FIG. 5F). Expression of genes important for mitochondrial function was restored by AAV-hTAZ, and cardiac stress markers were also normalized by the high dose treatment (FIGS. 5G to 5H). There was variable response in the medium dose group, with the subset of non-responsive mice continuing to have elevation of Myh7, Nppa, and Nppb (FIG. 5H). Due to the wide variation in the medium dose group, these measurements did not reach statistical significance by ANOVA. However, the reduction of Myh7 and Nppb were significant when the medium dose group were excluded. In addition to molecular markers, high dose AAV-hTAZ reduced cardiac fibrosis (FIGS. 5I to 5J) and apoptosis (FIGS. 5K to 5L) to normal levels. Medium dose AAV-hTAZ partially ameliorated abnormalities in these parameters, but they remained significantly elevated compared to control mice due to the subset with severe heart failure.

It is concluded that with sufficient cardiomyocyte transduction, AAV-hTAZ treatment can successfully prevent the development of cardiomyopathy.

### AAV-hTAZ Reversal of Established Cardiac Dysfunction

Many patients with BTHS will likely have established cardiac dysfunction by the time that gene therapy is considered. Therefore to test the efficacy and dose-response of AAV-hTAZ in a more clinically relevant context, serial echocardiograms on TAZ-CKO mice was performed, and enrolled mice when their shortening fraction was below 40% (typically around 2 months of age). Mice were then randomized to treatment with high dose AAV-hTAZ, medium dose AAV-hTAZ, or high dose AAV-Ctrl, where the high and medium doses were calibrated to transduced -70% and ~33% CM, respectively (FIG. 14A). After treatment, mice were followed by echocardiography monthly for 3 months. Hearts were then collected and subjected to molecular and histological studies.

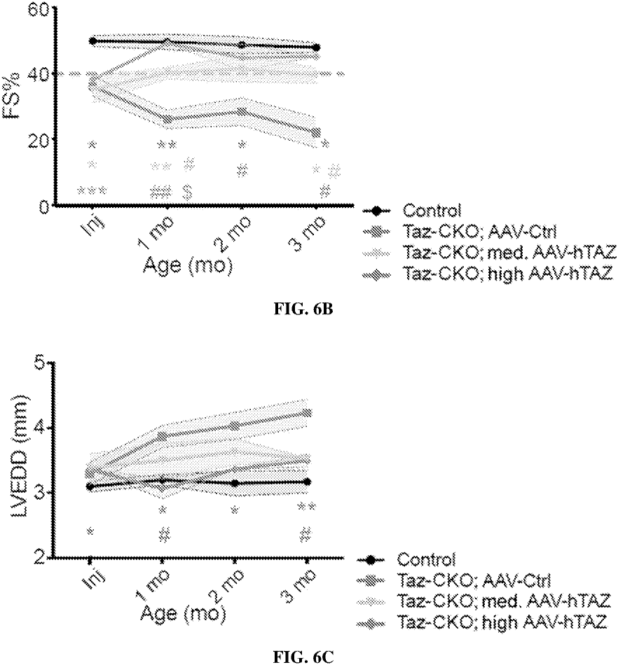

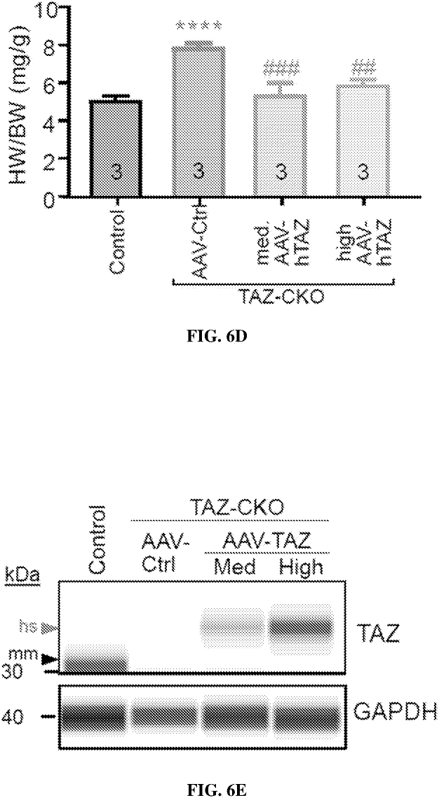

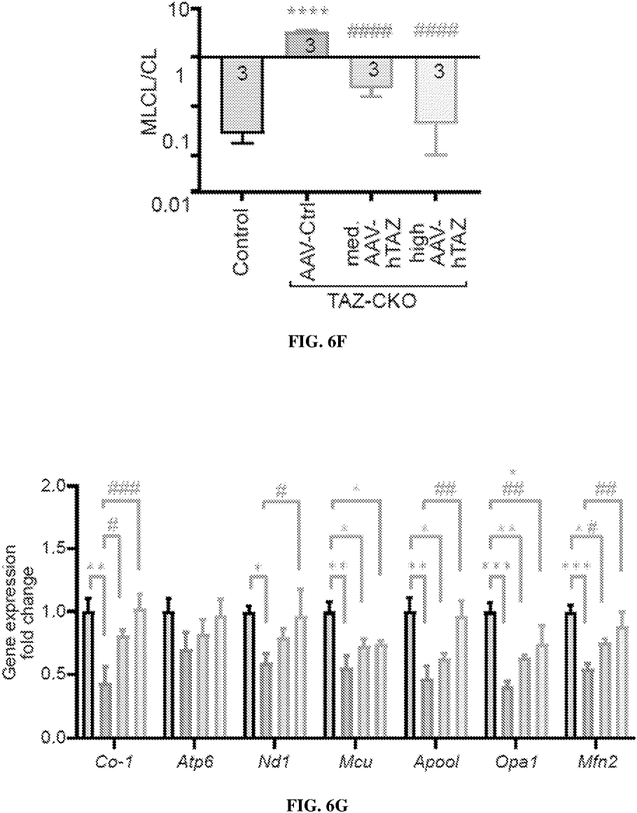

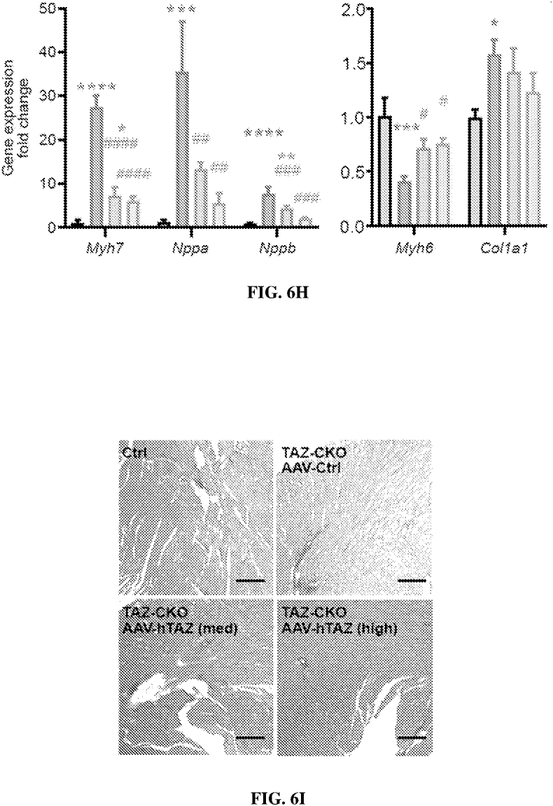

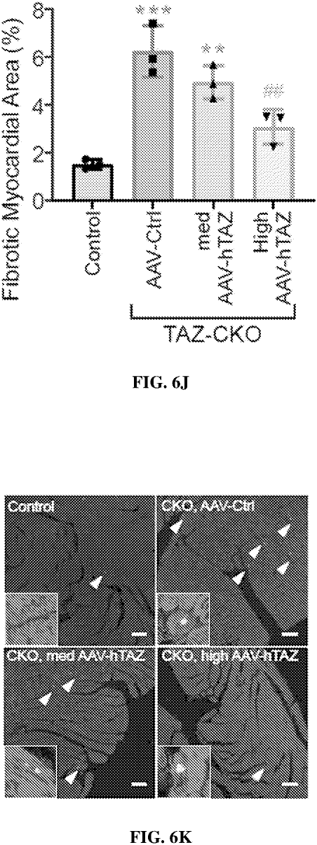

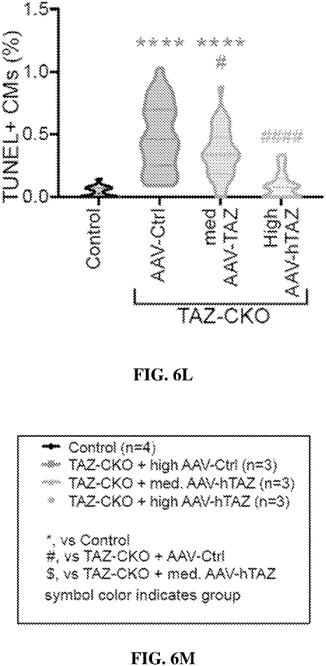

Monthly echocardiography demonstrated progressive deterioration of heart function with AAV-Ctrl treatment (FIG. 6B). In contrast, high dose AAV-hTAZ reverted cardiac function to normal levels, whereas the medium dose stabilized cardiac dysfunction (FIG. 6B). Both doses of AAV-hTAZ prevented LV dilatation (FIG. 6C). Cardiac hypertrophy observed in TAZ-CKO mice treated with AAV-Ctrl was not observed in medium and high dose AAV-hTAZ treated groups (FIG. 6D). Capillary western blotting of heart extracts prepared at the end of the study period confirmed loss of mTAZ in AAV-Ctrl treated TAZCKO mice, and dose-dependent replacement with full length human TAZ by AAV-hTAZ (FIG. 6E). Cardiolipin analysis confirmed dose-dependent correction of the MLCL/CL ratio by AAV-hTAZ (FIG. 6F). Expression of genes critical for mitochondrial function and morphology and cardiac stress markers were normalized by high dose AAV-hTAZ (FIGS. 6G to 6H). High dose AAV-hTAZ reduced but did not completely normalize cardiac fibrosis and cardiomyocyte apoptosis (FIGS. 6I to 6L), whereas the medium dose mildly reduced the cell death but did not significantly improve the overall fibrosis. Together, the results indicate that AAV-hTAZ administered at a sufficient dose reverses mild cardiomyopathy in TAZ-CKO hearts.

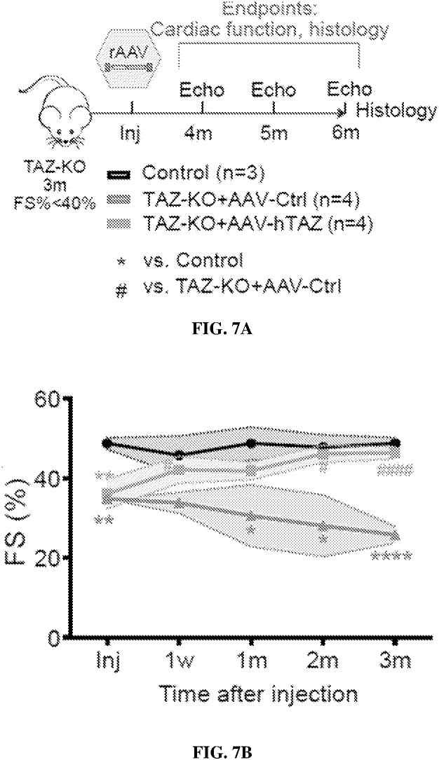

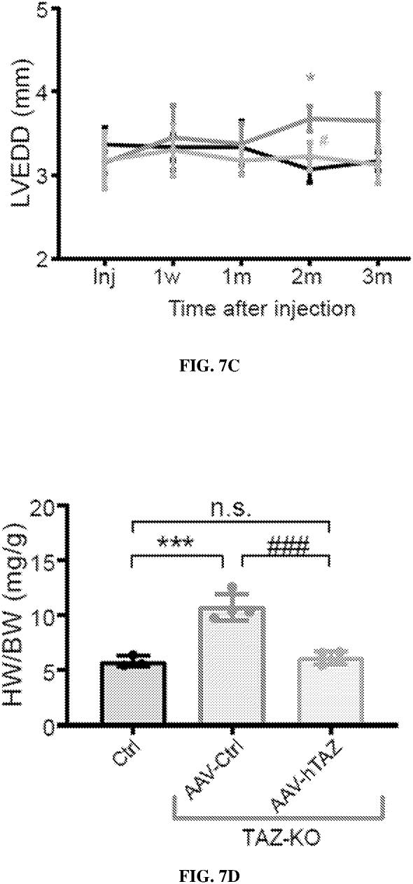

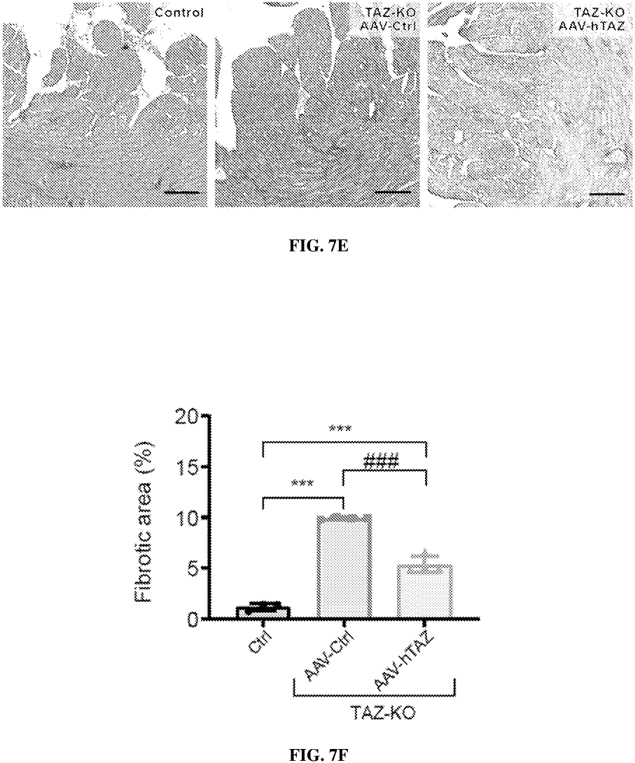

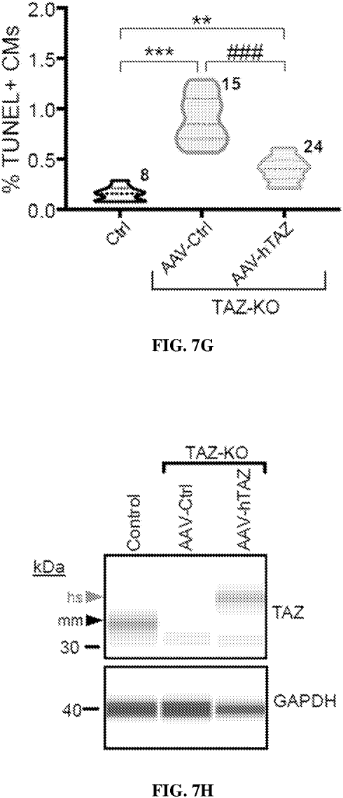

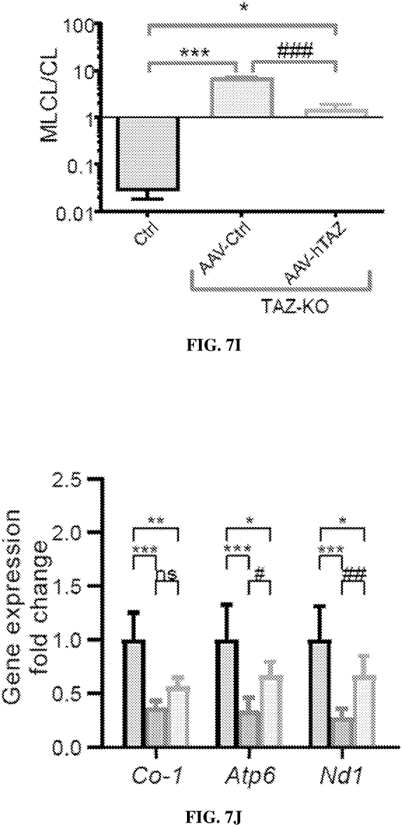

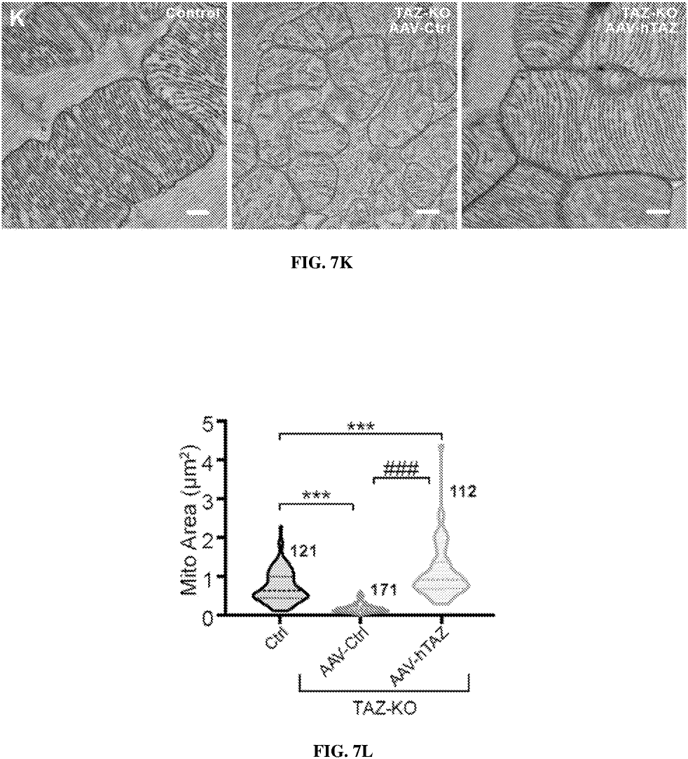

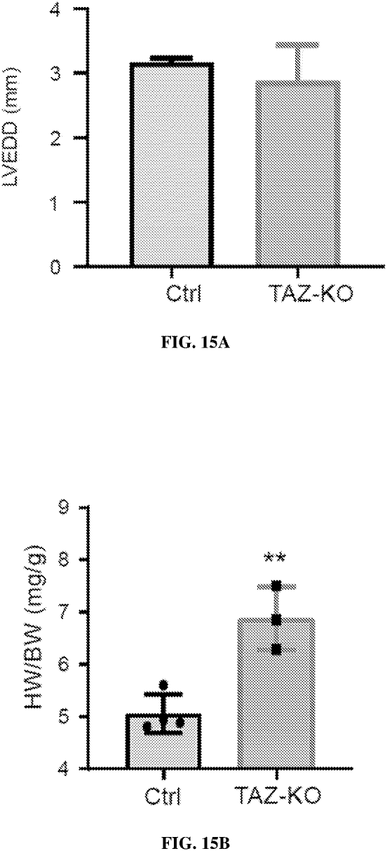

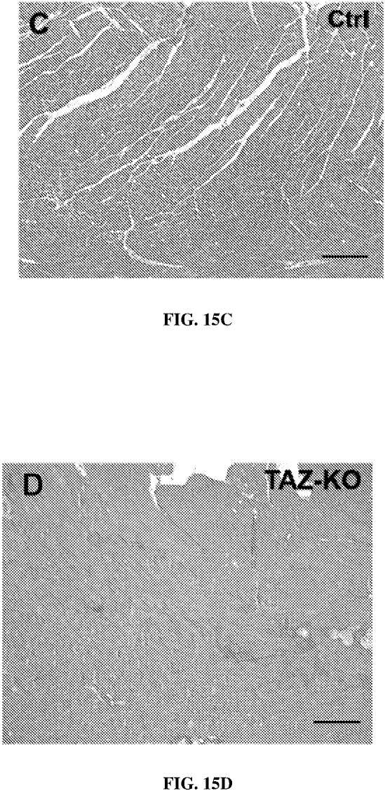

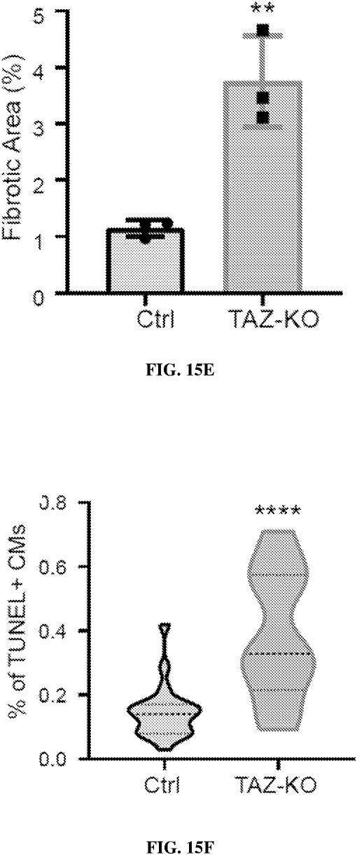

Because global TAZ knockout in TAZ-KO mice likely involves multiple organ systems, which may modify the effectiveness of AAV-hTAZ gene therapy for cardiomyopathy, it was then asked if AAV-hTAZ reverses established cardiomyopathy in the germline TAZ-KO model (FIG. 7A). Although most TAZ-KO mice die before weaning, through intensive breeding, there were enough TAZ-KO mice collected to study. In initial experiments, 3 month-old TAZ-KO mice were characterized, because this was the time point when most had mild LV dysfunction (FS% < 40%), hypertrophy and mild histological abnormalities (FIG. 7B and FIG. 15). A therapeutic trial was performed a to reverse established cardiac dysfunction. 3-month-old TAZ-KO mice with FS% < 40% were enrolled and received AAV-hTAZ or AAV-Ctrl (encoding luciferase) at a dose calibrated to transduce ~70% CMs and the transgene was found to remain stable in the heart for at least 90 days after injection (FIG. 14B, top panel). Mice underwent monthly echocardiography for 3 months, and then hearts were removed for further analysis.

AAV-hTAZ treated mice had progressive improvement in heart function, and by 3 months after treatment FS% was not significantly different from control mice (FIG. 7B). The LV was not dilated at the start of the trial and tended to become more dilated over time in AAV-Ctrl but not in AAV-hTAZ (FIG. 15A and FIG. 7C). AAV-hTAZ prevented cardiac hypertrophy, as assessed by the heart weight to body weight ratio (FIG. 7D). On histological sections, AAV-hTAZ reduced myocardial fibrosis compared to AAV-Ctrl, although the extent of fibrosis remained elevated compared to control mice (FIGS. 7E to 7F). AAV-hTAZ likewise reduced CM apoptosis compared to AAV-Ctrl, although the frequency of apoptotic CMs remained elevated compared to controls (FIG. 7G).

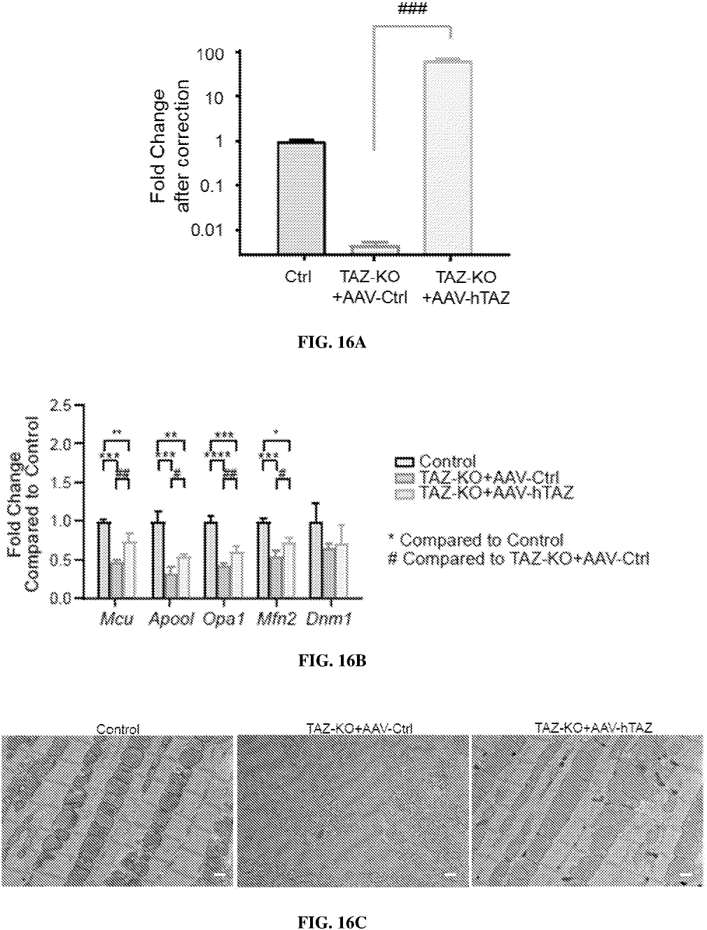

QRT-PCR was used and capillary western blotting to assess the extent of TAZ replacement (FIG. 7H and FIG. 16A). TAZ protein was restored to control levels by AAV-hTAZ (FIG. 7H). Analysis of cardiac cardiolipin showed that AAV-hTAZ markedly improved the MLCL/CL ratio compared to AAV-Ctrl (FIG. 7I). However, MLCL/CL remained elevated after AAV-hTAZ compared to the control genotype, consistent with transduction of most but not all cardiac cells.

Finally, mitochondrial ultrastructure and gene expression was evaluated. By qRT-PCR with Gapdh normalization, expression of transcripts related to mitochondria that are encoded in either the nuclear (FIG. 16B) or mitochondrial genomes (FIG. 7J) were depressed in TAZ-KO treated with AAV-Ctrl, and partially normalized by AAV-hTAZ. AAV-hTAZ improved mitochondrial morphology, increased the density and regularity of mitochondrial cristae (FIG. 7K), and normalized mitochondrial cross-sectional area (FIG. 7L). Abnormal clustering of mitochondria was similarly ameliorated in the treated group, and mitochondria were normally aligned along sarcomeres (FIG. 16C).

Overall, AAV-hTAZ reversed established cardiomyopathy, reduced cardiac fibrosis and cardiomyocyte apoptosis, and improved mitochondrial morphology and gene expression in the germline TAZ-KO model.

### Effect of AAV-hTAZ on Skeletal Muscle Phenotype

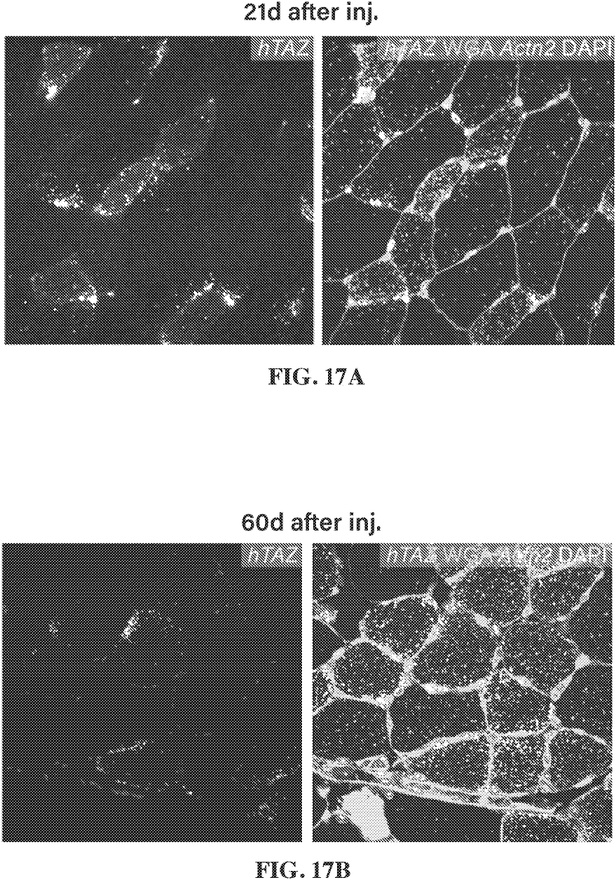

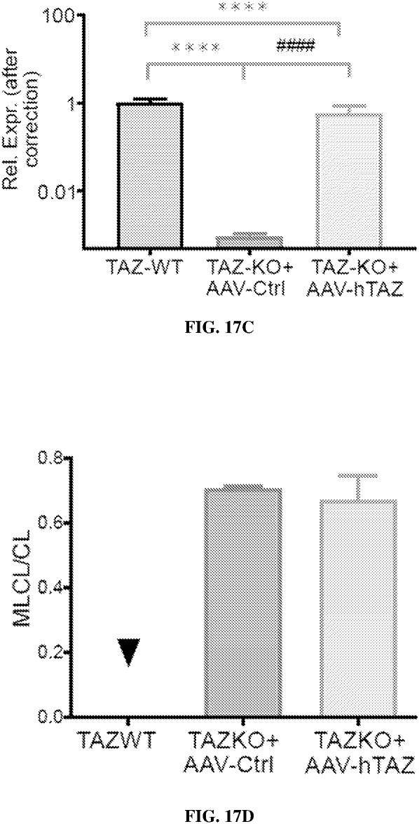

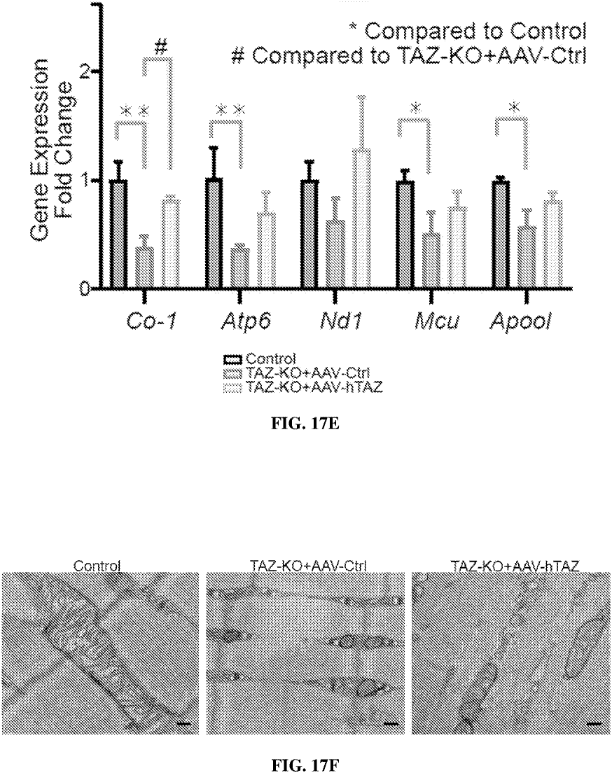

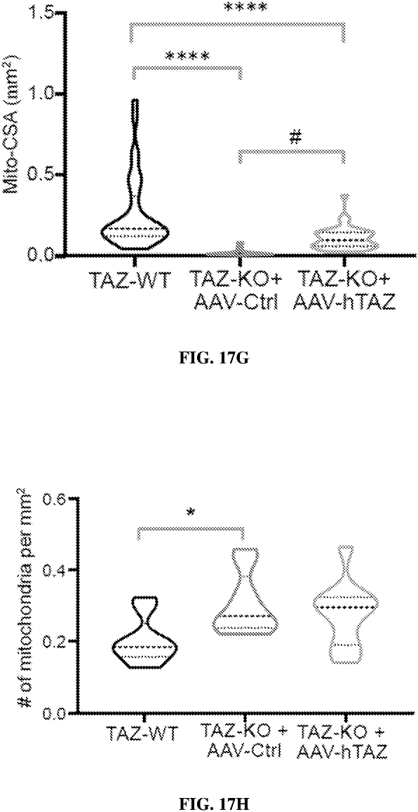

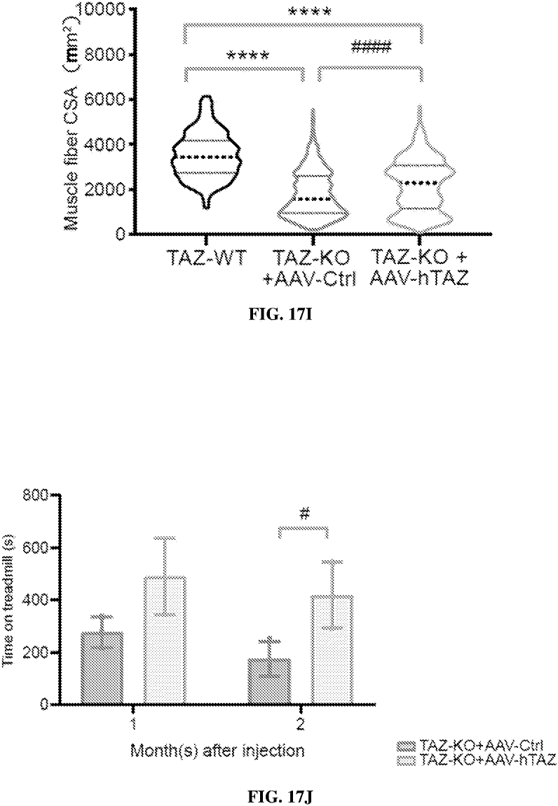

Though AAV9 high dose described above was able to efficiently transduce the heart and improve cardiac function, when administered to adult mice it was insufficient to comparably transduce skeletal muscle cells (27% in quadriceps) (FIG. 14B lower panel and FIG. 17A), probably due to the relative large skeletal muscle mass compared to the heart. Viral genome was further gradually lost from skeletal muscle over time, as the transduction efficiency was 17% at 60 days and 11% at 90 days after AAV administration (FIG. 14B and FIG. 17B). Comparable observations of viral genome dilution in skeletal muscle were made in several other gene therapy trials targeting skeletal muscle^{35,36}.

AAV-hTAZ increased expression of hTAZ transcripts in the quadriceps (FIG. 17C), although expression was low compared to hTAZ expression in heart (FIG. 13C). This relatively lower expression of hTAZ was correlated with no significant improvement of the MLCL/CL ratio (FIG. 17D). Nevertheless, AAV-hTAZ improved mitochondrial gene expression in skeletal muscle, although it did not restore it to normal (FIG. 17E). EM analysis (FIG. 17F) also showed that a subset of mitochondria in AAV-hTAZ treated mice had significantly improved cross-sectional area (FIG. 17G); however, mitochondrial area density was not significantly different between AAV-hTAZ and AAV-Ctrl groups (FIG. 17H). Morphological analysis showed that the average TAZ-KO muscle fiber cross sectional area was greater AAV-hTAZ compared to AAV-Ctrl (FIG. 17I). However, AAV-hTAZ did not restore muscle cross-sectional area to normal.

Finally, the exercise tolerance of TAZ-KO mice treated with AAV-hTAZ compared to AAV-Ctrl was examined using the maximal exercise treadmill test, described in FIGS. 11K to 11L. AAV-hTAZ improved exercise tolerance of TAZ-KO compared to AAV-Ctrl. The improvement tended towards significance at one month (p=0.057) was significant at two months (p=0.029; FIG. 17J). However, treated mice continued to have reduced exercise tolerance compared to WT mice, which could continue running for beyond the maximum test duration of 840 sec. This functional improvement may be correlated with the mild morphological and gene expression changes that was observed, despite the lack of improvement in the instantaneous CL profile.

Together, the results indicate the dose of AAV-hTAZ sufficient to correct cardiomyopathy had a mild but measurable salutary effect on skeletal muscle function in TAZ-KO mice.

### Discussion

Constitutive and cardiac specific TAZ knockout models were characterized, which had demonstrate recapitulate multiple features of BTHS, including: fetal and perinatal demise; poor feeding; growth retardation; progressive cardiac dysfunction; cardiac fibrosis; skeletal muscle weakness and hypoplasia; low neutrophil count; and impaired CL remodeling. The detailed characterization of the natural history of these models sets the stage for using them to evaluate the efficacy of potential BTHS therapies. Compared to the doxycycline-induced short hairpin RNA knockdown model reported previously^{19,20}, this model has clear advantages, including greater similarity to BTHS patients, the lack of residual TAZ, far less inter-individual variation, and freedom from high dose doxycycline, which itself can affect mitochondrial function²¹ and metalloprotease activity²².

A notable finding from this model is that cardiac fibrosis and cardiomyocyte apoptosis are important features of the BTHS-related cardiomyopathy. It was validated that human BTHS hearts that require transplantation also exhibited marked cardiac fibrosis, in both infants and adolescents. This is consistent with clinical findings in which heart failure symptoms can be more severe than would be expected by the degree of systolic dysfunction, suggestive of diastolic dysfunction. Additional studies are required to dissect the mechanisms that lead to cardiomyocyte apoptosis and cardiac fibrosis. Since cardiolipin interaction with cytochrome C regulates a key apoptotic trigger³⁹, a molecular pathway may link TAZ deficiency to CM apoptosis. Cell autonomous predisposition of TAZ deficient CMs to apoptosis has important implications for gene therapy, since non-transduced CMs would continue to be at risk for death.

The newly characterized models were used to evaluate the efficacy and dose-response of AAV-TAZ gene therapy for BTHS. A recent study used the shRNA TAZ knockdown mouse model to provide proof-of-concept that AAV-TAZ gene therapy might be effective for BTHS²³. However, as summarized in the introduction, this study left several important questions crucial for clinical translation unaddressed. Would gene replacement therapy work in a model without residual TAZ expression? What level of cardiomyocyte transduction is required for efficacy? Could TAZ gene therapy reverse established cardiac dysfunction? The study successfully addressed these questions. In both TAZ-KO and TAZCKO models, it was found that AAV-TAZ gene therapy rescued neonatal death, prevented cardiac dysfunction, and even reversed mild, established cardiac dysfunction. AAV-TAZ halted cardiac fibrosis and ameliorated CM apoptosis. Importantly, however, efficacy, consistency, and durability of therapy were contingent upon transduction of over 70% CMs. Transduction of 30-40% CMs resulted in partial and inconsistent efficacy which waned over several months, likely due to progressive disease in untransduced CMs. This finding is an important consideration for clinical translation -- whereas transduction of over 90% CMs is readily achievable with AAV9 in rodents, achieving sufficiently high CM transduction in humans or other large animals may be more challenging. Potential ways to overcome this hurdle are to develop improved vectors or administration methods so that transduction becomes more efficient⁴⁰, or to develop methods of immune modulation that will permit repeated dosing⁴¹.

Consistent with prior reports on the TAZ knockdown mouse model^{20,42} and human BTHS patients⁴³, the TAZ-KO model showed muscular defects. Though most TAZ-KO display neonatal lethality, some were able to survive to adulthood. Muscle samples from these mutants were found to have altered transcriptional profiles and morphological changes. Functionally, TAZ-KO mice that survived to adulthood had normal motor activity before exercise, had reduced motor activity after mild exercise, and had profoundly lower endurance under more strenuous exercise. AAV-hTAZ partially rescued this phenotype when administered to adult mice, despite low transduction efficiency and diminishing fraction of TAZ+ skeletal myocytes over time. However, further investigation of skeletal muscle responses to AAV-TAZ gene therapy will require use of conditional and inducible skeletal muscle deficient models, to circumvent limitations imposed by the high loss of TAZ-KO neonates. Such studies of the skeletal muscle abnormalities and their responses to therapy are beyond the scope of this study, but they are important to consider in translating AAV-based gene therapy to treating BTHS patients.

It was surprising to find that cardiac specific TAZ knockout did not result in fetal or neonatal lethality, whereas global TAZ knockout did. This suggested that lack of TAZ in cells other than CMs is responsible for the demise of TAZ-KO neonates. Indeed, cardiomyocyte-specific TAZ gene replacement directed by AAV2i8 and the cardiac-specific troponin T promoter failed to rescue TAZ-KO. In contrast, AAV2i8 and the striated muscle (heart plus skeletal muscle) specific synthetic MHCK7 promoter rescued neonatal demise of most TAZ-KO mice. These observations, in combination with the low gross motor activity of TAZ-KO neonates, their weight loss between P1 and P2, the infrequency of finding TAZ-KO neonates with milk spot, and the structural and functional abnormalities that were observed in skeletal muscles, strongly suggest that neonatal demise was due to weakness and poor feeding, coupled with low metabolic reserves due to intrauterine growth retardation. BTHS infants are hypotonic and often have feeding issues, although due to supportive care this is a not significant cause of mortality in human patients.

TABLE 1

Primers used in Example 1

| | | | |
|---|---|---|---|
| Target | | Sequence (5′-3′) | Application |
| TAZ | F | CCATGGGGACTGGGTGCAC | cloning |
| | R | TCTGCCTGCATCTTCAGCCG | cloning |
| mTaz | F | ATTGGACGGCTGATTGCTGAGTGT | QPCR |
| | R | AGTCTGTGAGGGCTTTCCGCATCT | QPCR |
| hTaz | F | GAGAACAAGTCGGCTGTG | QPCR |
| | R | GGCTGGAGGTGGTTGTGG | QPCR |
| Myh6 | F | AACCAGAGTTTGAGTGACAGAATG | QPCR |
| | R | ACTCCGTGCGGATGTCAA | QPCR |
| Myh7 | F | GCGACTCAAAAAGAAGGACTTTG | QPCR |
| | R | GGCTTGCTCATCCTCAATCC | QPCR |
| Nppa | F | GCTTCCAGGCCATATTGGAG | QPCR |
| | R | GGGGGCATGACCTCATCTT | QPCR |
| Nppb | F | GAGGTCACTCCTATCCTCTGG | QPCR |
| | R | GCCATTTCCTCCGACTTTTCTC | QPCR |
| Il1a | F | CGAAGACTACAGTTCTGCCATT | QPCR |
| | R | GACGTTTCAGAGGTTCTCAGAG | QPCR |
| Co-1 | F | TGAAACCCCCAGCCATAAC | QPCR |
| | R | GGGTGCCCAAAGAATCAGA | QPCR |
| Nd-1 | F | GCCCCCTTCGACCTGACA | QPCR |
| | R | CGGAAGCGTGGATAAGATGC | QPCR |
| Apt6 | F | CTCAAAACGCCTAATCAACAAC | QPCR |
| | R | TACGGCTCCAGCTCATAGTG | QPCR |
| Mcu | F | AAAGGAGCCAAAAAGTCACG | QPCR |
| | R | AACGGCGTGAGTTACAAACA | QPCR |
| Apool | F | ATGGCGGCCTTTAGGATGG | QPCR |
| | R | TCCGGTCTCACTAGCTGCT | QPCR |
| Opal | F | TGGAAAATGGTTCGAGAGTCAG | QPCR |
| | R | CATTCCGTCTCTAGGTTAAAGCG | QPCR |
| Mfn2 | F | AGAACTGGACCCGGTTACCA | QPCR |
| | R | CACTTCGCTGATACCCCTGA | QPCR |
| Dnm1 | F | TTACGGTTCCCTAAACTTCACG | QPCR |
| | R | GTCACGGGCAACCTTTTACGA | QPCR |
| Col1a1 | F | GCTCCTCTTAGGGGCCACT | QPCR |
| | R | CCACGTCTCACCATTGGGG | QPCR |
| Col3a1 | F | CTGTAACATGGAAACTGGGGAAA | QPCR |
| | R | CCATAGCTGAACTGAAAACCACC | QPCR |
| Taz-WT-F1 | F | CTTGCCCACTGCTCACAAAC | Genotyping |
| Taz-WT-R1 | R | CAGGCACATGGTCCTGTTTC | Genotyping |
| Taz-KO-R1 | R | CCAAGTTGCTAGCCCACAAG | Genotyping |
| Myh6-Cre | F | ATGACAGACAGATCCCTCCTATCTCC | Genotyping |
| | R | CTCATCACTCGTTGCATCATCGAC | Genotyping |
| AAV-Titer | F | CTCAAGGCTTTCACGCAGCCAC | AAV titration |
| | R | GGCATGAACATGGTTAGCAGAGGCTCTAG | AAV titration |

### REFERENCES

1. Clarke SLN, Bowron A, Gonzalez IL, Groves SJ, Newbury-Ecob R, Clayton N, Martin RP, Tsai-Goodman B, Garratt V, Ashworth M, Bowen VM, McCurdy KR, Damin MK, Spencer CT, Toth MJ, Kelley RI, Steward CG. Barth syndrome. Orphanet J Rare Dis. 2013;8:23.
2. Roberts AE, Nixon C, Steward CG, Gauvreau K, Maisenbacher M, Fletcher M, Geva J, Byrne BJ, Spencer CT. The Barth Syndrome Registry: distinguishing disease characteristics and growth data from a longitudinal study. Am J Med Genet A. 2012;158A:2726-2732.
3. Spencer CT, Byrne BJ, Bryant RM, Margossian R, Maisenbacher M, Breitenger P, Benni PB, Redfearn S, Marcus E, Cade WT. Impaired cardiac reserve and severely diminished skeletal muscle O₂ utilization mediate exercise intolerance in Barth syndrome. Am J Physiol Heart Circ Physiol. 2011;301:H2122-9.
4. Bione S, D’Adamo P, Maestrini E, Gedeon AK, Bolhuis PA, Toniolo D. A novel X-linked gene, G4.5. is responsible for Barth syndrome. Nat Genet. 1996;12:385-389.
5. Houtkooper RH, Turkenburg M, Poll-The BT, Karall D, Perez-Cerda C, Morrone A, Malvagia S, Wanders RJ, Kulik W, Vaz FM. The enigmatic role of tafazzin in cardiolipin metabolism. Biochim Biophys Acta. 2009;1788:2003-2014.
6. Xu Y, Malhotra A, Ren M, Schlame M. The enzymatic function of tafazzin. J Biol Chem. 2006;281:39217-39224.
7. Schlame M, Greenberg ML. Biosynthesis, remodeling and turnover of mitochondrial cardiolipin. Biochim Biophys Acta Mol Cell Biol Lipids. 2017;1862:3-7.
8. Mejia EM, Nguyen H, Hatch GM. Mammalian cardiolipin biosynthesis. Chem Phys Lipids. 2014;179:11-16.
9. Zhang M, Mileykovskaya E, Dowhan W. Gluing the respiratory chain together. Cardiolipin is required for supercomplex formation in the inner mitochondrial membrane. J Biol Chem. 2002;277:43553-43556.
10. Pfeiffer K, Gohil V, Stuart RA, Hunte C, Brandt U, Greenberg ML, Schägger H. Cardiolipin stabilizes respiratory chain supercomplexes. J Biol Chem. 2003;278:52873-52880.
11. Xu Y, Phoon CKL, Berno B, D’Souza K, Hoedt E, Zhang G, Neubert TA, Epand RM, Ren M, Schlame M. Loss of protein association causes cardiolipin degradation in Barth syndrome. Nat Chem Biol. 2016;12:641-647.
12. Schlame M, Kelley RI, Feigenbaum A, Towbin JA, Heerdt PM, Schieble T, Wanders RJ, DiMauro S, Blanck TJ. Phospholipid abnormalities in children with Barth syndrome. J Am Coll Cardiol. 2003;42:1994-1999.
13. Kulik W, van Lenthe H, Stet FS, Houtkooper RH, Kemp H, Stone JE, Steward CG, Wanders RJ, Vaz FM. Bloodspot assay using HPLC-tandem mass spectrometry for detection of Barth syndrome. Clin Chem. 2008;54:371-378.
14. Vreken P, Valianpour F, Nijtmans LG, Grivell LA, Plecko B, Wanders RJ, Barth PG. Defective remodeling of cardiolipin and phosphatidylglycerol in Barth syndrome. Biochem Biophys Res Commun. 2000;279:378-382.
15. Valianpour F, Mitsakos V, Schlemmer D, Towbin JA, Taylor JM, Ekert PG, Thorburn DR, Munnich A, Wanders RJA, Barth PG, Vaz FM. Monolysocardiolipins accumulate in Barth syndrome but do not lead to enhanced apoptosis. J Lipid Res. 2005;46:1182-1195.
16. Wang G, McCain ML, Yang L, He A, Pasqualini FS, Agarwal A, Yuan H, Jiang D, Zhang D, Zangi L, Geva J, Roberts AE, Ma Q, Ding J, Chen J, Wang D-Z, Li K, Wang J, Wanders RJA, Kulik W, Vaz FM, Laflamme MA, Murry CE, Chien KR, Kelley RI, Church GM, Parker KK, Pu WT. Modeling the mitochondrial cardiomyopathy of Barth syndrome with induced pluripotent stem cell and heart-on-chip technologies. Nat Med. 2014;20:616-623.
17. Gu Z, Valianpour F, Chen S, Vaz FM, Hakkaart GA, Wanders RJA, Greenberg ML. Aberrant cardiolipin metabolism in the yeast taz1 mutant: a model for Barth syndrome. Mol Microbiol. 2004;51:149-158.
18. Xu Y, Condell M, Plesken H, Edelman-Novemsky I, Ma J, Ren M, Schlame M. A Drosophila model of Barth syndrome. Proc Natl Acad Sci USA. 2006;103:11584-11588.
19. Phoon CKL, Acehan D, Schlame M, Stokes DL, Edelman-Novemsky I, Yu D, Xu Y, Viswanathan N, Ren M. Tafazzin knockdown in mice leads to a developmental cardiomyopathy with early diastolic dysfunction preceding myocardial noncompaction. J Am Heart Assoc [Internet]. 2012;1. Available from: http://dx.doi.org/10.1161/JAHA.111.000455 20. Soustek MS, Falk DJ, Mah CS, Toth MJ, Schlame M, Lewin AS, Byrne BJ. Characterization of a transgenic short hairpin RNA-induced murine model of Tafazzin deficiency. Hum Gene Ther. 2011;22:865-871.
21. Moullan N, Mouchiroud L, Wang X, Ryu D, Williams EG, Mottis A, Jovaisaite V, Frochaux MV, Quiros PM, Deplancke B, Houtkooper RH, Auwerx J. Tetracyclines Disturb Mitochondrial Function across Eukaryotic Models: A Call for Caution in Biomedical Research. Cell Rep. 2015;10:1681-1691.
22. Golub LM, Lee HM, Ryan ME, Giannobile WV, Payne J, Sorsa T. Tetracyclines inhibit connective tissue breakdown by multiple non-antimicrobial mechanisms. Adv Dent Res. 1998;12:12-26.
23. Suzuki-Hatano S, Saha M, Rizzo SA, Witko RL, Gosiker BJ, Ramanathan M, Soustek MS, Jones MD, Kang PB, Byrne BJ, Cade WT, Pacak CA. AAV-Mediated TAZ Gene Replacement Restores Mitochondrial and Cardioskeletal Function in Barth Syndrome. Hum Gene Ther [Internet]. 2018;Available from: http://dx.doi.org/10.1089/hum.2018.020
24. Greenberg B, Butler J, Felker GM, Ponikowski P, Voors AA, Desai AS, Barnard D, Bouchard A, Jaski B, Lyon AR, Pogoda JM, Rudy JJ, Zsebo KM. Calcium upregulation by percutaneous administration of gene therapy in patients with cardiac disease (CUPID 2): a randomised, multinational, double-blind, placebo-controlled, phase 2b trial. Lancet. 2016;387:1178-1186.
25. Ren M, Xu Y, Erdjument-Bromage H, Donelian A, Phoon CKL, Terada N, Strathdee D, Neubert TA, Schlame M. Extramitochondrial cardiolipin suggests a novel function of mitochondria in spermatogenesis. J Cell Biol. 2019;218:1491-1502.
26. Agah R, Frenkel PA, French BA, Michael LH, Overbeek PA, Schneider MD. Gene recombination in postmitotic cells. Targeted expression of Cre recombinase provokes cardiac-restricted, site-specific rearrangement in adult ventricular muscle in vivo. J Clin Invest. 1997;100:169-179.
27. Guo Y, Jardin BD, Zhou P, Sethi I, Akerberg BN, Toepfer CN, Ai Y, Li Y, Ma Q, Guatimosim S, Hu Y, Varuzhanyan G, VanDusen NJ, Zhang D, Chan DC, Yuan G-C, Seidman CE, Seidman JG, Pu WT. Hierarchical and stage-specific regulation of murine cardiomyocyte maturation by serum response factor. Nat Commun. 2018;9:3837.
28. Sun G, Yang K, Zhao Z, Guan S, Han X, Gross RW. Matrix-assisted laser desorption/ionization time-of-flight mass spectrometric analysis of cellular glycerophospholipids enabled by multiplexed solvent dependent analyte-matrix interactions. Anal Chem. 2008;80:7576-7585.
29. Dougherty JP, Springer DA, Gershengom MC. The Treadmill Fatigue Test: A Simple, High-throughput Assay of Fatigue-like Behavior for the Mouse. J Vis Exp [Internet]. 2016;Available from: http://dx.doi.org/10.3791/54052
30. Castro B, Kuang S. Evaluation of Muscle Performance in Mice by Treadmill Exhaustion Test and Whole-limb Grip Strength Assay. Bio Protoc [Internet]. 2017;7. Available from: http://dx.doi.org/10.21769/BioProtoc.2237
31. Cadalbert LC, Ghaffar FN, Stevenson D, Bryson S, Vaz FM, Gottlieb E, Strathdee D. Mouse Tafazzin Is Required for Male Germ Cell Meiosis and Spermatogenesis. PLoS One. 2015;10:e0131066.
32. Turgeon B, Meloche S. Interpreting neonatal lethal phenotypes in mouse mutants: insights into gene function and human diseases. Physiol Rev. 2009;89:1-26.
33. Ferrari FK, Samulski T, Shenk T, Samulski RJ. Second-strand synthesis is a rate-limiting step for efficient transduction by recombinant adeno-associated virus vectors. J Virol. 1996;70:3227-3234.
34. McCarty DM. Self-complementary AAV vectors; advances and applications. Mol Ther. 2008;16:1648-1656.
35. Le Hir M, Goyenvalle A, Peccate C, Précigout G, Davies KE, Voit T, Garcia L, Lorain S. AAV genome loss from dystrophic mouse muscles during AAV-U7 snRNA-mediated exonskipping therapy. Mol Ther. 2013;21:1551-1558.
36. Vulin A, Barthélémy I, Goyenvalle A, Thibaud J-L, Beley C, Griffith G, Benchaouir R, le Hir M, Unterfinger Y, Lorain S, Dreyfus P, Voit T, Carlier P, Blot S, Garcia L. Muscle function recovery in golden retriever muscular dystrophy after AAV1-U7 exon skipping. Mol Ther. 2012;20:2120-2133.
37. Asokan A, Conway JC, Phillips JL, Li C, Hegge J, Sinnott R, Yadav S, DiPrimio N, Nam H-J, Agbandje-McKenna M, McPhee S, Wolff J, Samulski RJ. Reengineering a receptor footprint of adeno-associated virus enables selective and systemic gene transfer to muscle. Nat Biotechnol. 2010;28:79-82.
38. Salva MZ, Himeda CL, Tai PW, Nishiuchi E, Gregorevic P, Allen JM, Finn EE, Nguyen QG, Blankinship MJ, Meuse L, Chamberlain JS, Hauschka SD. Design of tissue-specific regulatory cassettes for high-level rAAV-mediated expression in skeletal and cardiac muscle. Mol Ther. 2007;15:320-329.
39. Kagan VE, Tyurin VA, Jiang J, Tyurina YY, Ritov VB, Amoscato AA, Osipov AN, Belikova NA, Kapralov AA, Kini V, Vlasova II, Zhao Q, Zou M, Di P, Svistunenko DA, Kurnikov IV, Borisenko GG. Cytochrome c acts as a cardiolipin oxygenase required for release of proapoptotic factors. Nat Chem Biol. 2005;1:223-232.
40. Colella P, Ronzitti G, Mingozzi F. Emerging Issues in AAV-Mediated In Vivo Gene Therapy. Mol Ther Methods Clin Dev. 2018;8:87-104.
41. Vandamme C, Adjali O, Mingozzi F. Unraveling the Complex Story of Immune Responses to AAV Vectors Trial After Trial. Hum Gene Ther. 2017;28:1061-1074.
42. Powers C, Huang Y, Strauss A, Khuchua Z. Diminished Exercise Capacity and Mitochondrial bc1 Complex Deficiency in Tafazzin-Knockdown Mice. Front Physiol. 2013;4:74.
43. Hornby B, McClellan R, Buckley L, Carson K, Gooding T, Vernon HJ. Functional exercise capacity, strength, balance and motion reaction time in Barth syndrome. Orphanet J Rare Dis. 2019;14:37.

### Example 2. hTAZ Isoform 2 (hTAZ Del5) Is Effective in Treating Barth Syndrome

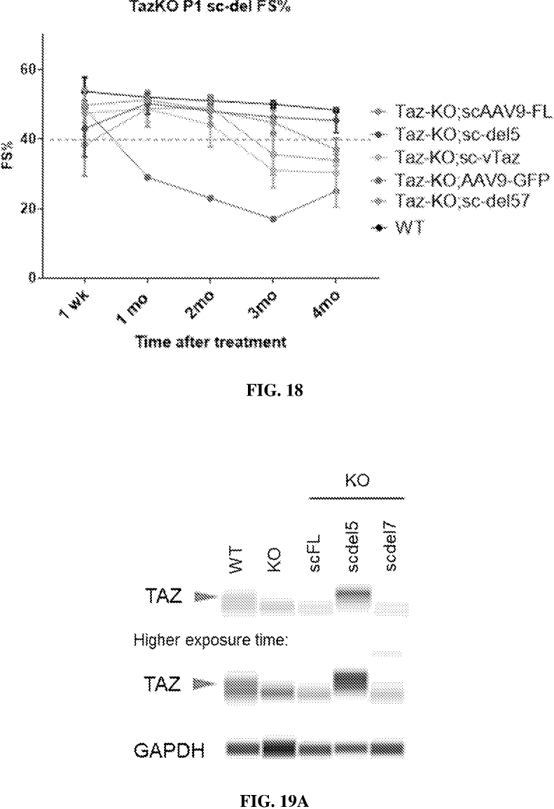

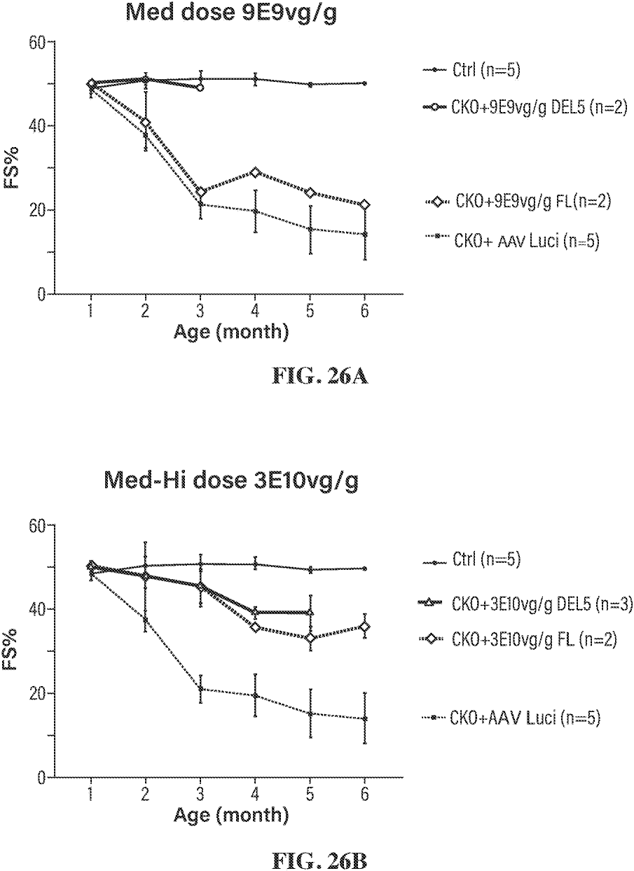

hTAZ isoforms were also tested for their activity in protecting TAZ-KO mice against cardiac dysfunction using the methods as described in Example 1. As shown in FIG. 18, neonatal TAZ-KO mice were treated with recombinant adeno-associated virus (AAV) expressing different naturally existing isoforms of human Tafazzin. Similar dosages were given with each virus and the resulted effect of cardiac protection is shown by the comparison to AAV-GFP treated TAZ-KO mice. The result shows that hTAZ-del5 (hTAZ isoform 2) achieved comparable activity as the full-length TAZ. In dose response experiments, high doses of del5 and full length TAZ showed similar beneficial effects on heart function (FIG. 26B). However, at lower doses the del5 isoform demonstrated greater efficacy (FIG. 26A).

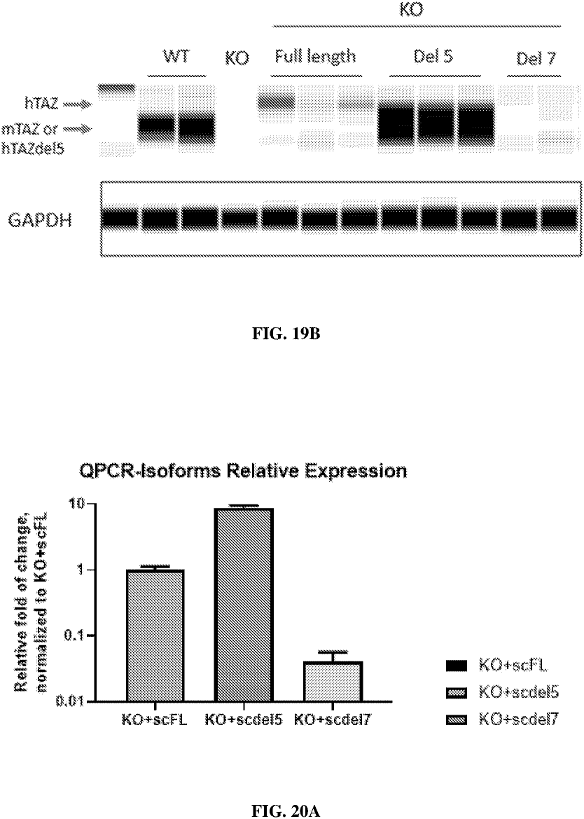

Protein levels of different hTAZ isoforms were also examined in protein extract of the heart. Cardiac samples were collected from scAAV-hTAZ treated TAZ-KO mice 10 days after injection and analyzed using capillary western blotting. hTAZ del5 was expressed to a significantly higher level compared to other isoforms (FIGS. 19A and 19B).

Relative expression of different hTAZ transcripts in the heart after AAV-hTAZ treatment were also evaluated. Cardiac samples were collected 10 days after injection and examined with qRT-PCR. Expression levels were normalized to Gapdh and compared to full-length TAZ group. Levels of viral genome were also examined via qPCR using primers targeting the promoter sequence of the viral genome. Amount of viral genome was normalized to genomic Gapdh and represented as in comparison to full-length TAZ group. The result shows that hTAZ-del5 transcript level is much higher than hTAZ-del7 (FIG. 20A), while the level of viral genome is comparable in all tested samples (FIG. 20B).

### Example 3. hTAZ Isoform 2 (hTAZ Del5) Reduces Signs of Disease When Expressed in Barth Syndrome Human Cells

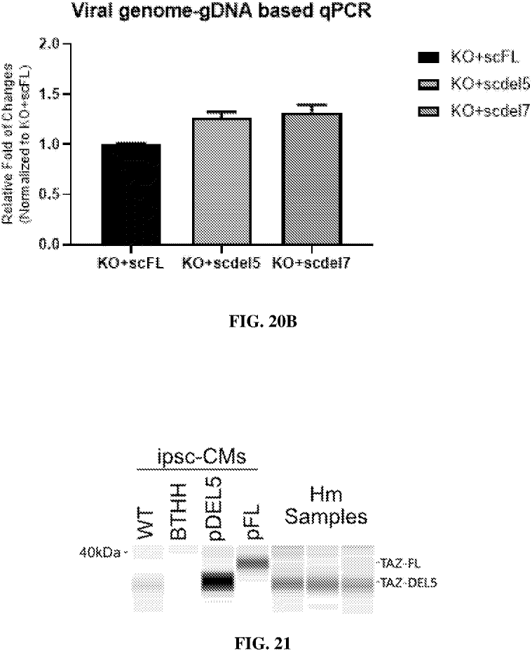

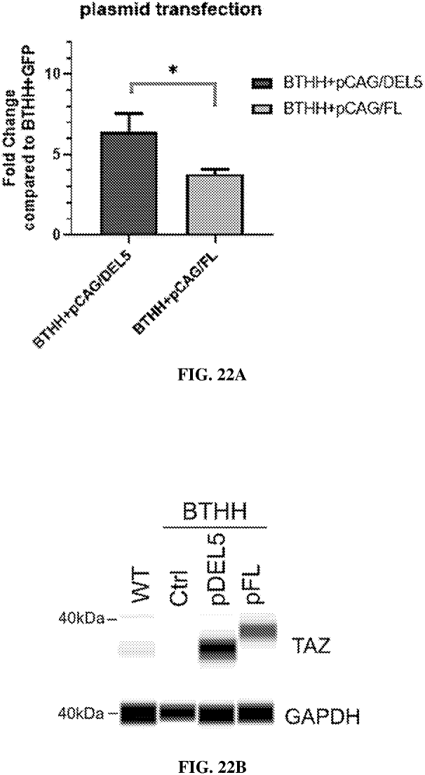

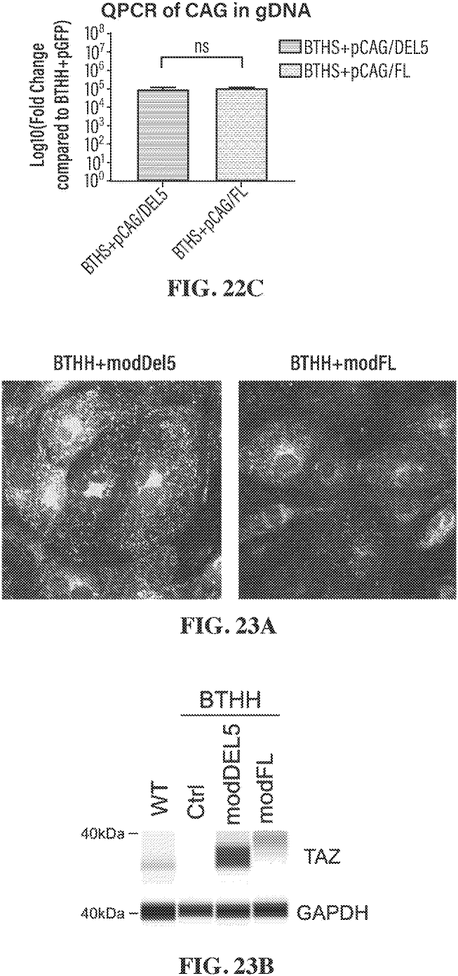

hTAZ isoforms were next tested for expression and efficacy in human myocardiac cells. As shown in FIG. 21, expression of hTAZ isoforms measured in human myocardium. For reference, the assay included iPSC-derived CMs expressing WT TAZ, iPSC-derived CMs expressing a frameshift variant of TAZ occurring in Barth patients (BTHH), and iPSC-derived CMs with either full length hTAZ (FL) or hTAZ-del5 overexpressed from a plasmid. As observed previously in protein extracts from mouse hearts (FIGS. 19A and 19B), hTAZ-del5 was expressed to a higher level than FL hTAZ. Expression of hTAZ in iPSC-derived CMs was further compared to that of samples extracted from human patient hearts. The molecular weight of hTAZ endogenously expressed in human myocardium most most closely matches that of hTAZ-del5, and little full length hTAZ protein was detected. Further analysis of hTAZ expression in iPSC-derived CMs indicated that hTAZ-del5 mRNA transcripts were also expressed to a significantly higher level than those of FL hTAZ (FIGS. 22A and 22B). This significantly higher level of mRNA expression could not be correlated to any difference in level of transfection (FIG. 22C).

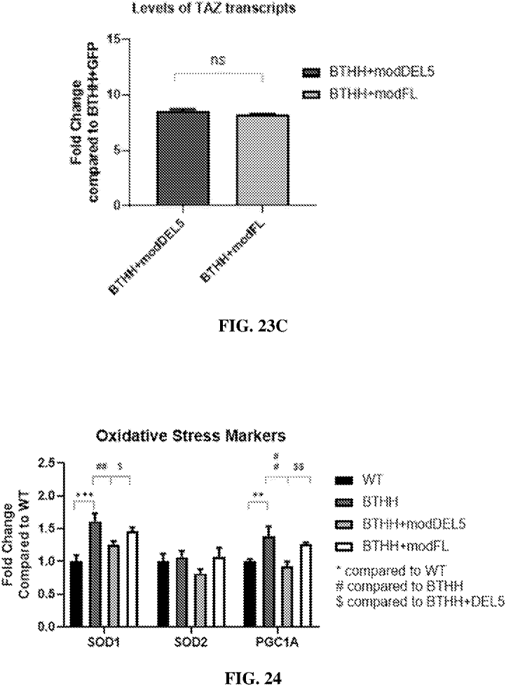

Expression of hTAZ isoforms in human cells was next assessed using a fluorescent approach. Modified RNA encoding del5-P2A-mCherry and FL-P2A-mCherry hTAZ isoforms was transfected into BTHH iPSC-CMs and assessed (FIG. 23A). Expression of del5-P2A-mCherry was substantially higher than FL-P2A-mCherry, as determined by fluorescent imaging (FIG. 23A) and capillary western blotting (FIG. 23B). Consistent with previous findings, the differences observed in expression of FL hTAZ and hTAZ-del5 could not be correlated to differences in mRNA cells received by transfection (FIG. 23C).

BTHH iPSC-CMs transfected with modified mRNAs encoding either FL hTAZ or hTAZ-del5 were then evaluated for changes in certain functional deficiencies characteristic of Barth syndrome. Changes in oxidative stress was assessed by measuring the expression level of antioxidative defense genes by qPCR 2 days after modified RNA transfection (FIG. 24). Transfection with modified RNA encoding hTAZ-del5 (modDel5) significantly reduced expression of SOD1 and PGC1A in BTHH iPSC-CMs, to a level more similar to WT iPSC-CMs. Full length hTAZ (modFL) modestly reduced expression, but to a level that remained significantly higher than that of transfection with modDel5. These findings indicate that hTAZ-del5 can effectively reduce oxidative stress in human cells with Barth syndrome.

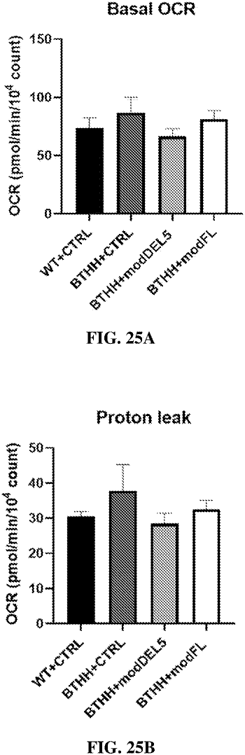

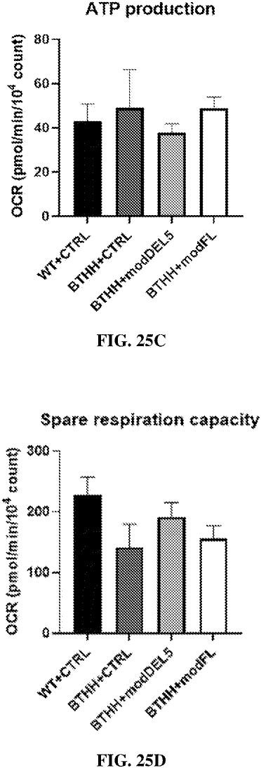

BTHH iPSC-CMs were further assessed for changes in mitochondrial respiratory function when transfected with hTAZ-del5 or FL hTAZ. BTHH mutant cells have altered respiration capacity, exacerbated basal oxygen consumption rate, proton leak, and ATP production compared to WT (FIGS. 25A to 25D), transfection with modDel5 completely or partially reversed each of these changes. Consistent with previous findings, transfection with modFL had a more modest effect, if any, upon these measures of mitochondrial respiration. The data indicates that the Del5 TAZ isoform is better than the FL isoform at improving mitochondrial function.

Next the dose response for transfection with these isoforms was subsequently explored in a cardiac specific TAZ knockout mouse model (CKO). Neonatal CKO mice were transfected with varying levels of AAV encoding either with hTAZ-del5 or FL hTAZ and cardiac contraction was assessed by echocardiography every month for 6 months. When administered at a dose of 9E9 vg/g (FIG. 26A), mice receiving AAV-FL exhibited no improvement over mice receiving AAV-Luciferase. Those that received AAV-DEL5 however showed no loss of cardiac function over time compared to healthy controls. When a higher dose of 3E10 vg/g was administered (FIG. 26B), both AAV-FL and AAV-DEL5 protected CKO hearts from the onset of disease over 4 months, although mice receiving AAV-DEL5 exhibited somewhat higher heart contractile function. Together these data indicate that both hTAZ-del5 or FL hTAZ isoforms can prevent the onset of Barth syndrome, though hTAZ-del5 is the more efficacious of the two isoforms.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browserexecutable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A nucleic acid molecule comprising a nucleotide sequence encoding a human Tafazzin (hTAZ) isoform comprising an amino acid sequence that is at least 90% identical to SEQ ID NO: 2.

2. The nucleic acid molecule of claim 1, wherein the hTAZ isoform comprises the amino acid sequence of SEQ ID NO: 2.

3. The nucleic acid of claim 1 or claim 2, wherein the nucleotide sequence encoding the hTAZ isoform is operably linked to a promoter.

4. The nucleic acid molecule any one of claims 1-3, wherein the nucleic acid molecule is a vector.

5. The nucleic acid molecule of claim 4, wherein the vector is a viral vector for expressing the hTAZ isoform.

6. The nucleic acid molecule of claim 5, wherein the viral vector is selected from a lentiviral vector, a retroviral vector, or a recombinant adeno-associated virus (rAAV) vector.

7. The nucleic acid molecule of claim 6, wherein the viral vector is a rAAV vector further comprising two AAV inverted terminal repeats (ITRs) flanking the nucleotide sequence encoding the hTAZ isoform and the promoter.

8. The nucleic acid molecule of any one of claims 1-7, wherein the nucleotide sequence encoding the hTAZ isoform is at least 90% identical to SEQ ID NO: 4.

9. The nucleic acid molecule of claim 8, wherein the nucleotide sequence encoding the hTAZ isoform comprises SEQ ID NO: 4.

10. The nucleic acid molecule of any one of claims 1-7, wherein the nucleotide sequence encoding the hTAZ isoform is codon-optimized.

11. The nucleic acid molecule of claim 10, wherein the nucleotide sequence encoding the hTAZ isoform is at least 90% identical to SEQ ID NO: 6.

12. The nucleic acid molecule of claim 11, wherein the nucleotide sequence encoding the hTAZ isoform comprises SEQ ID NO: 6.

13. The nucleic acid molecule of claim 1 or claim 2, wherein the nucleic acid is a messenger RNA (mRNA).

14. The nucleic acid molecule of claim 13, wherein the mRNA is a modified mRNA.

15. The nucleic acid molecule of claim 13 or claim 14, wherein the mRNA comprises a nucleotide sequence that is at least 90% identical to SEQ ID NO: 27.

16. The nucleic acid molecule of claim 15, wherein the mRNA comprises the nucleotide sequence of SEQ ID NO: 27.

17. A recombinant adeno-associated virus (rAAV) comprising a capsid protein and the nucleic acid molecule of any one of claims 6-12.

18. The rAAV of claim 17, wherein the capsid protein is of a serotype selected from AAV1, AAV2, AAV2i8, AAV3, AAV4, AAV5, AAV6, AAV6.2, AAV7, AAV8, AAV9, AAV.rh8, AAV.rh10, AAV.rh39, AAV.rh74, AAV.43, AAV2/2-66, AAV2/2-84, and AAV2/2-125, or a variant thereof.

19. The rAAV of claim 18, wherein the capsid protein is of a serotype AAV9 or AAV2i8.

20. The rAAV of any one of claims 17 to 19, wherein the capsid protein comprises the sequence set forth in any one of SEQ ID NOs: 7-23 and 28.

21. The rAAV of any one of claims 17-20, wherein the rAAV is a self-complementary AAV (scAAV).

22. A composition comprising the nucleic acid molecule of any one of claims 1-16, or the rAAV of any one of claims 17-21.

23. The composition of claim 22, further comprising a pharmaceutically acceptable carrier.

24. Use of the nucleic acid molecule of any one of claims 1-16, the rAAV of any one of claims 17-21, or the composition of claim 22 or claim 23 for treating Barth syndrome (BTHS).

25. Use of the nucleic acid molecule of any one of claims 1-16, the rAAV of any one of claims 17-21, or the composition of claim 22 or claim 23 for improving cardiac or skeletal muscle function.

26. Use of the nucleic acid molecule of any one of claims 1-16, the rAAV of any one of claims 17-21, or the composition of claim 22 or claim 23 for enhancing cardiolipin biogenesis.

27. A method of treating Barth syndrome (BTHS), the method comprising administering to a subject in need thereof an effective amount of a hTAZ isoform comprising an amino acid sequence that is at least 90% identical to SEQ ID NO: 2, the nucleic acid molecule of any one of claims 1-16, the rAAV of any one of claims 17-21, or the composition of claim 22 or claim 23.

28. A method of improving cardiac or skeletal muscle function, the method comprising administering to a subject in need thereof an effective amount of a hTAZ isoform comprising an amino acid sequence that is at least 90% identical to SEQ ID NO: 2, the nucleic acid molecule of any one of claims 1-16, the rAAV of any one of claims 17-21, or the composition of claim 22 or claim 23.

29. A method of treating cardiac or skeletal muscle disease, the method comprising administering to a subject in need thereof an effective amount of a hTAZ isoform comprising an amino acid sequence that is at least 90% identical to SEQ ID NO: 2, the nucleic acid molecule of any one of claims 1-16, the rAAV of any one of claims 17-21, or the composition of claim 22 or claim 23.

30. A method of enhancing cardiolipin biogenesis, the method comprising administering to a subject in need thereof an effective amount of a hTAZ isoform comprising an amino acid sequence that is at least 90% identical to SEQ ID NO: 2, the nucleic acid molecule of any one of claims 1-16, the rAAV of any one of claims 17-21, or the composition of claim 22 or claim 23.

31. The method of any one of claims 27-30, wherein the subject is human.

32. The method of any one of claims 27-31, wherein the administering is via injection.

33. The method of any one of any one of claims 27-32, wherein the hTAZ isoform comprises the amino acid sequence of SEQ ID NO: 2.

34. The method of claim 33, wherein the hTAZ isoform is administered.

35. The method of any one of claims 27-32, wherein the nucleic acid molecule is administered.

36. The method of any one of claims 27-32, wherein the rAAV is administered.

37. The method of any one claims 27-32, wherein the composition is administered.

38. A method of treating Barth syndrome (BTHS), the method comprising administering to a subject in need thereof an effective amount of a recombinant adeno-associated virus (rAAV), wherein the AAV comprises a capsid protein of serotype AAV9 and a nucleotide sequence encoding a human Tafazzin (hTAZ) isoform comprising the amino acid sequence of SEQ ID NO: 2, wherein the nucleotide sequence comprises SEQ ID NO: 6 and is operably linked to a promoter, and wherein the nucleotide sequence and the promoter are flanked by AAV inverted terminal repeats (ITRs).

39. The method of claim 38, wherein the rAAV is a self-complementary recombinant adeno-associated virus (scAAV).
